(19) United States
(12) Patent Application Publication
(10) Pub. No.: US 2023/0098772 A1
(43) Pub. Date: Mar. 30, 2023

(54) ADVANCED MICROBIOME THERAPEUTICS ENGINEERED TO PRODUCE SEROTONIN IN VIVO

(71) Applicants: DANMARKS TEKNISKE UNIVERSITET, Kgs. Lyngby (DK); THE TRUSTEES OF COLUMBIA UNIVERSITY IN THE CITY OF NEW YORK, New York, NY (US)

(72) Inventors: Morten Otto Alexander Sommer, Hellerup (DK); Mareike Bongers, Frederiksberg (DK); Harris He Wang, New York, NY (US); Frank Cusimano, Richardson, TX (US)

(21) Appl. No.: 17/617,918

(22) PCT Filed: Jun. 12, 2020
(86) PCT No.: PCT/EP2020/066383

§ 371 Date: Dec. 9, 2021

Related U.S. Application Data
(60) Provisional application No. 62/861,007, filed on Jun. 13, 2019.

(30) Foreign Application Priority Data
Jul. 17, 2019 (EP) 19186680.5

(51) Int. Cl. C12P 17/10; A61K 38/44; A61K 38/45; A61K 38/51; A61P 25/22; A61P 25/24; C12N 9/02; C12N 9/10; C12N 9/88

(52) U.S. Cl. CPC C12P 17/10; A61K 38/44; A61K 38/45; A61K 38/51; A61P 25/22; A61P 25/24; C12N 9/0071; C12N 9/1003; C12N 9/1029; C12N 9/88; C12Y 114/16004; C12Y 201/01004; C12Y 203/01087; C12Y 305/04016; C12Y 401/01028

(57) ABSTRACT

The invention provides a composition for use as a medicament, comprising cells of a recombinant microorganism capable of producing increased amounts of one or more of 5-hydroxytryptophan (5-HTP), 5-hydroxytryptamine (5-HT) and tryptamine (TRM) as compared to the non-recombinant microorganism from which it was derived. The composition finds use in preventing and/or treating TRM-; 5-HTP-, or 5-HT-related disorders of the central nerve system (CNS); enteric nervous system (ENS); gastro intestine (GI) and metabolism in a mammal, and may be orally administered to a mammal in need thereof. Additionally, a composition comprising cells of a recombinant microorganism capable of producing melatonin is provided for use as a medicament, such as for treatment of depression, dementia, cancer and sleep disorder.

### FIELD OF THE INVENTION

The invention provides a composition for use as a medicament, comprising cells of a recombinant microorganism capable of producing increased amounts of one or more of 5-hydroxytryptophan (5-HTP), 5-hydroxytryptamine (5-HT) and tryptamine (TRM) as compared to the non-recombinant microorganism from which it was derived. The composition finds use in preventing and/or treating TRM-; 5-HTP-, or 5-HT-related disorders of the central nerve system (CNS); enteric nervous system (ENS); gastro intestine (GI) and metabolism in a mammal, and may be orally administered to a mammal in need thereof. Additionally, a composition comprising cells of a recombinant microorganism capable of producing melatonin is provided for use as a medicament.

### BACKGROUND OF THE INVENTION

The biogenic monoamine serotonin (5-hydroxytryptamine, 5-HT) is crucial for neurotransmission and many other functions throughout the body. Up to 95% of the serotonin in the body is produced in the gastrointestinal tract. Serotonin is derived from tryptophan (TRP) in a two-step reaction, first by a tryptophan hydroxylase (TPH) to form the 5-hydroxytryptophan (5-HTP) intermediate and then by tryptophan decarboxylase (TDC), as illustrated in FIG. 1. In humans, tryptophan hydroxylase is the rate-limiting step of 5-HT biosynthesis and is made in two isoforms, TPH1 and TPH2. TPH2 is produced in neurons across the body, while TPH1 is predominately expressed in neuroendocrine cells in the gut.

In the GI tract, serotonin regulates intestinal motility, cell turnover, and homeostasis. In the brain, an imbalance in the serotonin level is linked to anxiety and depression. Treatment includes strategies for elevating serotonin in a patient suffering from any of the wide range of anxiety- and depression-related diseases.

Selective serotonin reuptake inhibitors (SSRIs) are widely prescribed for the treatment of depression. They are believed to increase the extracellular level of the neurotransmitter serotonin by limiting its reabsorption (reuptake) into the presynaptic cell, increasing the level of serotonin in the synaptic cleft available to bind to the postsynaptic receptor. A sub-group of patients present symptoms that are SSRI-treatment resistant (TRD). A treatment of TRD patients, currently under clinical trial in mammalian models, is based on a combination therapy of an SSRI (serotonin re-uptake inhibitor) and the serotonin precursor, 5-hydroxytryptophan (5-HTP). This has undesirable consequences, due to the rapid uptake of administered 5-HTP, leading to sudden spikes in 5-HT levels. Efforts to address this problem currently focus on combination therapies employing slow-release forms of 5-HTP.

Intestinal bacteria play a crucial role in gastrointestinal (GI) health and homeostasis. Beyond modulating gut homeostasis, intestinal microbes are thought to participate in a bidirectional signaling pathway with the enteric nervous system (ENS) and the central nervous system (CNS) along the gut-brain axis. Resident microbiota in the gut are known to send signals to host mucosal endocrine cells to maintain gut 5-HT content and 5-HT plasma levels, rather than themselves providing a significant supply of 5-HT. More recently, it has been shown that oral ingestion of certain bacteria in animal models leads to changes in host physiology and behavior, implicating a role of these microbes in gut-brain communication.

In the Hippocampus, striatum, cortex, and dentate gyrus of the brain, serotonin-based biosynthesis pathways, serotonin-based receptors, and serotonin-based signaling pathways are implicated in memory, cognitive/age-related spatial learning, and memory formation. As serotonergic afferent neurons from the gastrointestinal tract alters dorsal motor nucleus of the vagus and nucleus of the tractus solitarius through the vagus nerve, intestinal 5-HT is implicated in neuronal memory, cognition, and learning.

In view of the key role of TRM and 5-HT in mammalian homeostasis and medical conditions arising from insufficient 5-HT levels, there exists a need for new treatments for elevating the level of 5-HT in patients, in particular in those suffering from anxiety-related diseases, as well as TRD.

The signaling molecule melatonin is a highly pleiotropic molecule that is released as a hormone of the pineal gland predominantly during night. Melatonin secretion decreases during aging, but reduced melatonin levels are also observed in various diseases, such as types of dementia, some mood disorders, severe pain, cancer, and diabetes type 2. Melatonin dysfunction is frequently related to deviations in amplitudes, phasing, and coupling of circadian rhythms. Accordingly there exists a need for treatments that will elevate the level of melatonin in patients in need thereof.

### SUMMARY OF THE INVENTION

In a first embodiment, the invention provides a composition for use as a medicament, wherein said composition comprises cells of a recombinant microorganism, and wherein said microorganism comprises one or more recombinant nucleic acid molecules encoding one of more proteins selected from:

(a) tryptophan 5-hydroxylase (EC 1.14.16.4),

(b) tryptophan decarboxylase (EC 4.1.1.28), and

(c) tryptamine 5-hydroxylase (EC:1.14.-.-)

The cells are therefore capable of producing increased amounts of one or more of 5-hydroxytryptophan, 5-hydroxytryptamine and tryptamine as compared to cells of the non-recombinant microorganism from which it was derived.

In a second embodiment, the invention provides a recombinant bacterial cell comprising one or more recombinant nucleic acid molecules or transgenes encoding one of more proteins selected from:

(a) tryptophan 5-hydroxylase (EC 1.14.16.4),

(b) tryptophan decarboxylase (EC 4.1.1.28), and

(c) tryptamine 5-hydroxylase (EC:1.14.-.-)

wherein said microorganism is devoid of genes capable of expressing

trp operon repressor protein and tryptophanase (EC:4.1.99.1). The cell is therefore capable of producing increased amounts of one or more of 5-hydroxytryptophan, 5-hydroxytryptamine and tryptamine as compared to a cell of the non-recombinant microorganism from which it was derived.

Preferably, the recombinant bacterial cell is selected from among Escherichia, Bacteroides, Clostridium, Feacalibacterium, Eubacterium, Ruminococcus, Peptococcus, Peptostreptococcus, Lactobacillus, Lactococcus, Bifidobacterium, Enterococcus, Streptococcus, Pediococcus, Leuconostoc, Staphylococcus and Bacillus.

In a third embodiment the invention provides a method for preventing and/or treating a TRM-; 5-HTP-, 5-HT-; or melatonin-related disorder in a subject, the method comprising administering to the subject diagnosed with a TRM-; 5-HTP-; 5-HT-; or melatonin-related disorder recombinant bacteria engineered to express one or more:

(a) tryptophan 5-hydroxylase (EC 1.14.16.4),

(b) tryptophan decarboxylase (EC 4.1.1.28), or

(c) tryptamine 5-hydroxylase (EC:1.14.-.-)

The cells are therefore capable of producing increased amounts of one or more of 5-hydroxytryptophan, 5-hydroxytryptamine and tryptamine as compared to cells of the non-recombinant microorganism from which it was derived.

The TRM-; 5-HTP-, 5-HT-; or melatonin-related disorder may be a disorder of the central nerve system (CNS); enteric nervous system (ENS); gastro intestine (GI) or metabolism in a mammal.

In a fourth embodiment the invention provides a recombinant microorganism comprising one or more recombinant nucleic acid molecules or transgenes,

wherein said microorganism comprises recombinant nucleic acid molecules encoding:

(a) tryptophan 5-hydroxylase (EC 1.14.16.4), and tryptophan decarboxylase (EC 4.1.1.28), or

(b) tryptophan decarboxylase (EC 4.1.1.28) and tryptamine 5-hydroxylase (EC:1.14.-.-), and

recombinant nucleic acid molecules encoding:

(c) serotonin acetyltransferase (AANAT) EC 2.3.1.87 and

(d) acetylserotonin O-methyltransferase (ASMT) (EC 2.1.1.4),

wherein the cells are capable of producing melatonin. The cells are therefore capable of producing melatonin in amounts that are increased as compared to cells of the non-recombinant microorganism from which it was derived.

In a fifth embodiment, the invention provides a composition for use as a medicament, wherein said composition comprises cells of a recombinant microorganism capable of producing melatonin according to the fourth embodiment; suitable for oral delivery in a patient suffering from a melatonin-deficient or melatonin-related disorders, such as a the following melatonin-related disorders: circadian rhythm disturbance, insomnia, jet lag, autism; dementia, mood disorders, severe pain, cancer, and diabetes type 2.

### DESCRIPTION OF THE INVENTION

### BRIEF DESCRIPTION OF THE FIGURES

FIG. 1 Cartoon showing the catalytic steps of the 5-HT and melatonin biosynthetic pathway. TrpR (tryptophan operon repressor protein); TnaA (tryptophanase); FolE (encodes mutant GTP cyclohydrolase 1 (T198I)) TRP (tryptophan); TRM (tryptamine); 5-HTP (5-Hydroxytryptophan); 5-HT (serotonin); TPH (tryptophan hydroxylase); TDC (tryptophan decarboxylase); T5H (tryptamine 5-hydroxylase); serotonin acetyltransferase (AANAT) and acetylserotonin O-methyltransferase (ASMT).

FIG. 2 [A]: Histogram showing titers of TRM (tryptamine); 5-HTP (5-Hydroxytryptophan); 5-HT (serotonin) measured in culture media derived from cultures of cells of six E. coli Nissle strains engineered to co-express respectively: TPH and TDC genes encoding Human TPH1 and rice TDC (H1R); human THP2 (H2) and rice TDC (H2R); human THP2 (H2) and Cataranthus roseus TDC (H2C); human THP2 (H2) and Guinea pig TDC (H2G); mouse TPH1 (M1) and rice TDC (M1R); and mouse THP2 (M2) and rice TDC (M2R). [B]: Histogram showing titers of TRM (tryptamine); 5-HTP (5-Hydroxytryptophan); 5-HT (serotonin) measured in culture media derived from 5-HTP-fed cultures of cells of three E. coli Nissle strains engineered to co-express respectively: TPH and TDC genes encoding human THP2 (H2) and rice TDC (H2R); human THP2 (H2) and Cataranthus roseus TDC (H2C); human THP2 (H2) and Guinea pig TDC (H2G).

FIG. 3 Histogram showing titers of TRM (tryptamine); 5-HTP (5-Hydroxytryptophan); and 5-HT (serotonin) measured in culture media following cultivation of an E. coli Nissle strain (EcNΔ2+pUC-H2R) co-expressing TPH and TDC in growth media supplemented with the substrate, in the range of 10-100 mg tryptophan/L.

FIG. 4 [A] Graph showing cell density of cultures of four E. coli Nissle strains, measured as units of OD_{600 nm}. The strains are EcNΔ2+pUC-H2R; EcNΔ3+pUC-H2R; EcNΔ2+pMUT-H2R, each co-expressing human TPH2 (H2) and rice TDC (R); and EcN+pMUT empty plasmid. EcNΔ2 has deletions: ΔtrpR, ΔtnaA; EcNΔ3 has deletions ΔtrpR, ΔtnaA, ΔinfA. [B] Graph showing cell density of cultures of nine E. coli Nissle strains co-expressing human TPH2 (H2) or mouse TPH1 (M1) with rice TDC (R); and control strains comprising empty plasmids, measured as units of OD_{600 nm}. [C] Graph showing percent kanamycin resistant colonies detected in cultures of E. coli Nissle strains comprising pUC-based or pMUT-based serotonin production plasmids measured over a period of 100 generations of growth without antibiotic selection. At each time point, cells were plated on LB Agar plates+/−kanamycin, and ratios of colonies were calculated (n=6). [D] Histogram showing titers of 5-HT (serotonin) measured in culture media following cultivation of E. coli Nissle strains co-expressing human TPH2 (H2) and rice TDC (R) cloned in pUC (pUC-H2R) or pMUT (pMUT-H2R) plasmids for 0-100 cell generations.

FIG. 5 [A] Histogram showing titers of TRM (tryptamine) and 5-HT (serotonin) measured in culture media following cultivation of E. coli Nissle strain N; NΔ2 and “oN” co-expressing human TPH2 and rice TDC (H2R), or mouse TPH1 and rice TDC (M1R) operons in pUC or pMUC plasmids, or carrying an empty pMUT plasmid. [B] Histogram showing titers of TRM (tryptamine), 5-HTP (5-hydroxytryptophan) and 5-HT (serotonin) measured in culture media following cultivation of E. coli Nissle “oN” strains expressing mouse TPH1 (oN10=oN+pMUTM1); rice TDC (oN11=oN+pMUTR); mouse TPH1 and rice TDC (oN14=oN+pMUTM1R) or carrying an empty plasmid (oN9=pMUT). All data shown are mean values with error bars of standard deviation from n=3 biological replicates [C] and [D] left panels: Histogram showing titers of TRP (tryptophan); TRM (tryptamine); 5-HTP (5-Hydroxytryptophan); and 5-HT (serotonin) measured in culture media following cultivation of an E. coli Nissle strain comprising plasmids pMUT-H2R and pMUT-M1R functionally linked to RBS sequences having a range of translation strengths. Right panels: 2-Dimensional diagram showing yields of 5-HT; 5-HTP and tryptamine yields in cultivation medium measured for E. coli Nissle strain comprising a plasmid pMUT-H2R functionally linked to an RBS sequence having a range of translation strengths, x-axis corresponds to RBS strength for TPH gene; the y-axis corresponds to RBS strength for TDC; crossed out boxes indicate combinations not tested. [E] Cartoon showing genetic modifications to E. coli Nissle genome and genetic structure of the plasmids tested in [B]. [F] Histogram showing titers of 5-HTP measured in culture media following cultivation of E. coli Nissle strain NΔ2(=folE WT) or oN(=folE(T1981I), each comprising the human TPH2 pMUT plasmid.

FIG. 6 [A] Histogram showing titers of 5-HT (serotonin) and TRM (tryptamine) measured in culture media following cultivation of E. coli Nissle strain NΔ2 or strain oN each co-expressing human TPH2 and rice TDC (H2R) genes functionally linked to RBS sequences of different translation strengths cloned in a pUC plasmids. [B] Histogram showing titers of 5-HT (serotonin) and TRM (tryptamine) measured in culture media following cultivation of E. coli Nissle strain NΔ2 or strain oN each expressing a human TPH2 or mouse TPH1 gene in combination with a rice TDC (H2R) gene functionally linked to RBS sequences of different translation strengths cloned in pMUT plasmids. [C] Graph showing theoretical 5-HT concentration in the mouse gut relative to the abundance of 5-HT producing bacteria measured as a percent of the total gut microbiome population. Theoretical physiological levels of 5-HT in the gut lie in the range of 5-30 mg/L as indicated. The relative abundance of 5-HT producing bacteria required to produce 5-HT within the therapeutically effective range is plotted for bacteria having an in vivo 5-HT production rate (P) of 0.1 to 10 mg/L*OD₆₃₀*h.

FIG. 7 [A] Image of thin sections of mid-colon derived from mice 24 hours after last oral gavage with E. coli strains oN9, oN11, oN14 and control (PBS), where the sections were stained with DAPI and anti-GFP antibody. [B] Graph showing the kanamycin-resistant, GFP positive bacterial concentration in the duodenum, jejunal-ileal junction, cecum, proximal colon, distal colon, and fecal matter derived from mice 24 hours after last oral gavage with either E. coli strain oN9, oN11, or oN14, or control (PBS). Samples plated on LB Agar plates plus kanamycin and colonies counted measured as colony forming units (CFU) per gram of fecal matter (n=6). Data points shown at y=0 were below the level of detection of 10³ CFU/gram.

FIG. 8 Histogram showing [A] tryptophan and [B] 5-HT concentration measured as mg/L in the serum, mg/g biomass in cleaned homogenized colon tissue, mg/g biomass of fecal material (n=7-8) derived from mice 24 hours after last oral gavage with either E. coli strain oN9, oN11, or oN14, or control (PBS). All p-values shown are from one-way ANOVA with Tukey's post hoc correction for multiple comparison testing. All other comparisons not shown were not significant with p>0.05. Data are mean values with error bars as standard error of the mean. [C] Image of thin sections of mid-colon derived from mice 24 hours after last oral gavage with E. coli strains oN9, oN11, oN14 and control (PBS), where the sections were stained with DAPI and anti-serotonin antibody, where the scale bar is 100 μm.

FIG. 9 [A] Histogram showing relative expression of MUC2, TPH1, TPH2, and SERT gene expression levels, measured as relative mRNA by ΔΔCτ (n=6), in colonic tissue derived from mice 24 hours after last oral gavage with either E. coli strain oN9, oN11, or oN14, or control (PBS). Data are mean values with error bars as standard error of the mean. [B] Image of thin sections of mid-colon derived from mice 24 hours after last oral gavage with E. coli strains oN9, oN11, oN14 and control (PBS), where the sections were stained with DAPI and anti-Muc-2 antibody, where the scale bar is 400 μm. The arrow indicates increased MUC2 signal in oN14.

FIG. 10 Histograms [(a)-(e)] showing relative expression of 5-HT receptors HTR1B HTR1D, HTR3, HTR4, and HTR7 in the colon by RT-PCR (n=6), in colonic tissue derived from mice 24 hours after last oral gavage with either E. coli strain oN9, oN11, or oN14, or control (PBS). Histograms (f) showing total gastrointestinal transit time from mouth to anus using a 6% Carmine Red solution (Methods) (n=7-8) in correspondingly treated mice cohorts. All p-values shown are from one-way ANOVA with Tukey's post hoc correction for multiple comparison testing. All other comparisons not shown were not significant with p>0.05. Data are mean values with error bars as standard error of the mean.

FIG. 11 Histograms showing (a) time spent immobile of mice during the last four minutes of a six-minute Forced Swim Test (FST) (n=9-10); (b) number of fecal pellets (# of fecal boli) produced during a 10-minute Open Field Test (OFT) (n=7-10); (c) average total distance traveled by mice during the 10-minute OFT (n=7-10); (d) time spent in both the inner 40 cm×40 cm zone or outer 10 cm ring during OFT (n=7-10); and (e) representative tracking pattern of a single mouse from each mouse cohort after oral gavage with either E. coli strain oN9, oN11, or oN14, or control (PBS). All p values shown are from one-way ANOVA with Tukey's post hoc correction for multiple comparison testing. All other comparisons not shown were not significant with p>0.05. Data are mean values with error bars as standard error of the mean.

FIG. 12 Histograms showing (a) number of fecal boli produced during a 10-minute Open Field Test (OFT) (n=7-10); (b) time spent by mice in the inner 40 cm×40 cm zone during OFT (n=7-10); (c) average distance traveled by mice in the inner 40 cm×40 cm zone during the 10-minute OFT (n=7-10); (d) number of entries by mice into the inner 40 cm×40 cm zone during the 10-minute OFT (n=7-10); (e) average total distance traveled by mice during the 10-minute OFT (n=7-10); (f) time spent immobile of mice during the last four minutes of a six-minute Forced Swim Test (FST) (n=9-10); as measured for each mouse cohort after 21 days administration of Gavage: PBS/IP: saline (negative control); Gavage: PBS/IP: Fluoxetine (SSRI positive comparator), Gavage: E. coli strain oN14/IP: saline (oN14 positive comparator); and Gavage: E. coli strain oN14/IP: Fluoxetine (positive combination comparator). All p values shown are from one-way ANOVA with Tukey's post hoc correction for multiple comparison testing. All other comparisons not shown were not significant with p>0.05. Data are mean values with error bars as standard error of the mean.

FIG. 13 Histograms showing (A) 5-HT concentration and (B) tryptophan concentration in peripheral plasma samples, C) 5-HT concentration and D) 5-HTP concentration in urine samples from mice after oral gavage with either E. coli strain oN10 or E. coli strain (EcN-control) lacking TPH expression; (n=8, p<0.001 by two-tailed t-test for plasma 5-HT).

FIG. 14 Cartoon showing the structure of plasmids for expression of a serotonin pathway in S. cerevisiae (pSc-1 to pSc-3). RTDC=tryptophan decarboxylase gene (Rice); ck-TDC=tryptophan decarboxylase gene (Candidatus Koribacter versatilis Ellin345); TPH=tryptophan 5-hydroxylase 1 (mouse).

FIG. 15 (A) Histogram showing titers of 5-HT (serotonin) measured in culture media following cultivation of S. cerevisiae with or without plasmids expressing mouse TPH1 and and TDC genes from either rice (Sc-1 and Sc-2) or from Candidatus Koribacter versatilis Ellin345 (Sc-3). (B) Histogram showing titers of 5-HT (serotonin) and TRM (tryptamine) measured in culture media following cultivation of E. coli Nissle strain oN co-expressing mouse TPH1 and TDC genes from either rice (oN14) or from Candidatus Koribacter versatilis Ellin345 (M1ck). (C) Graph showing cell density of cultures of four E. coli Nissle strains, measured as units of OD_{600 nm}. oN control contains pMh-empty, oN14 contains pMUT14-5HT, M1R (trc)=mouse TPH1 and rice TDC expressed from trc promoter, M1ck (trc)=mouse TPH1 and ck-TDC expressed from trc promoter [SEQ ID No.: 112].

FIG. 16 Cartoon showing the structure of a plasmid for expression of a serotonin pathway in B. subtilis (pBS-M1Rf). R-TDC=tryptophan decarboxylase gene (Rice); TPH=tryptophan 5-hydroxylase 1 gene (mouse), FolE=mutant GTP cyclohydrolase I gene (GCH1).

### ABBREVIATIONS, TERMS AND DEFINITIONS

By “non-recombinant microorganism from which it was derived” is meant a microorganism which does not comprise the recombinant nucleic acid molecules.

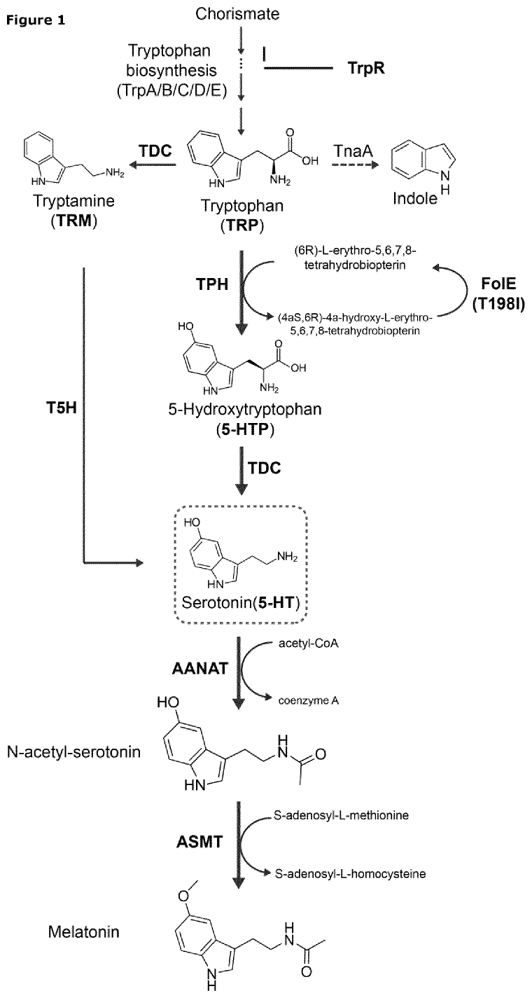

By “producing increased amounts” is meant compared to the non-recombinant organism without the recombinant nucleic acid molecules. For example, recombinant microbial cells expressing the recombinant enzymes of the claims are compared with microbial cells that do not comprise nucleic acid molecules for the enzymes in the claims. The cells with the recombinant nucleic acids encoding the enzymes will produce increased amounts of one or more of 5-hydroxytryptophan, 5-hydroxytryptamine and tryptamine as a result of expression of the encoded nucleic acids which express enzymes in the biosynthesis pathway according to FIG. 1.

gi number: (genInfo identifier) is a unique integer which identifies a particular sequence, independent of the database source, which is assigned by NCBI to all sequences processed into Entrez, including nucleotide sequences from DDBJ/EMBL/GenBank, protein sequences from SWISS-PROT, PIR and many others.

Amino acid sequence identity: The term “sequence identity” as used herein, indicates a quantitative measure of the degree of homology between two amino acid sequences of substantially equal length. The two sequences to be compared must be aligned to give a best possible fit, by means of the insertion of gaps or alternatively, truncation at the ends of the protein sequences. The sequence identity can be calculated as ((Nref−Ndif)100)/(Nref), wherein Ndif is the total number of non-identical residues in the two sequences when aligned and wherein Nref is the number of residues in one of the sequences. Sequence identity calculations are preferably automated using the BLAST program e.g. the BLASTP program (Pearson W. R and D. J. Lipman (1988)) (www.ncbi.nlm.nih.gov/cgi-bin/BLAST). Multiple sequence alignment is performed with the sequence alignment method ClustalW with default parameters as described by Thompson J., et al 1994, available at http://www2.ebi.ac.uk/clustalw/.

Preferably, the numbers of substitutions, insertions, additions or deletions of one or more amino acid residues in the polypeptide as compared to its comparator polypeptide is limited, i.e. no more than 1, 2, 3, 4, 5, 6, 7, 8, 9, or 10 substitutions, no more than 1, 2, 3, 4, 5, 6, 7, 8, 9, or 10 insertions, no more than 1, 2, 3, 4, 5, 6, 7, 8, 9, or 10 additions, and no more than 1, 2, 3, 4, 5, 6, 7, 8, 9, or 10 deletions. Preferably the substitutions are conservative amino acid substitutions: limited to exchanges within members of group 1: Glycine, Alanine, Valine, Leucine, Isoleucine; group 2: Serine, Cysteine, Selenocysteine, Threonine, Methionine; group 3: proline; group 4: Phenylalanine, Tyrosine, Tryptophan; Group 5: Aspartate, Glutamate, Asparagine, Glutamine.

Devoid of a gene encoding a functional protein: a microorganism that is devoid of a gene capable of expressing a functional protein (e.g. tryptophan repressor or tryptophanase) is a microorganism that either lacks the respective gene, or the gene id is modified (e.g inactivated) such that it is not able to express a functional protein. A range of genetic modifications are suitable for inactivating a gene, including the deletion of the gene (knockout) from the genome of a microbial cell; deletion of its cognate regulatory sequences (e.g. promoter); substitution of, or addition of, at least one nucleotide leading to a loss of expression of a functional polypeptide encoded by the gene. Where the encoded polypeptide is an enzyme, the genetic modification leads to a loss of detectable enzymatic activity of the respective polypeptide in the microbial cell.

Genome: is the genetic material present in a cell or organism; said genome comprising all of the information needed to build and maintain that cell or organism; and includes the genetic material in both chromosome(s) and plasmid(s) present within the cell or organism.

Mutant GTP cyclohydrolase I (EC 3.5.4.16): herein abbreviated as mutant GCH1, is to be understood as a mutant GCH1 that provides for an increased hydroxylation activity of a tryptophan 5-hydroxylase (EC 1.14.16.4) of at least 2-fold (for example: at least 3-fold, or 4-fold, or 5-fold, 6-fold or, 7-fold or, 8-fold or, 9-fold or, 10-fold), as compared to a non-mutant GCH1, as measured by 5-hydroxytryptophan yield. The non-mutant GCH1 can be the parent enzyme from which the mutant was derived, whereby mutation(s) found in the mutant confer the increased activity.

Native gene: endogenous gene in a microorganism cell genome, homologous to host microorganism.

RBS: Ribosomal Binding Site is a sequence of nucleotides upstream of the start codon of an mRNA transcript that is responsible for the recruitment of a ribosome during the initiation of protein translation.

Ribosome Binding Site (RBS) Calculator: provides a method for either predicting or controlling translation initiation rates (TIRs) in bacteria. When used to control translation of a given coding sequence, the RBS calculator generates synthetic DNA sequences that will result in a defined translation initiation rate (and therefore, protein expression strength). For any promoter sequence (controlling transcription strength), many different RBS strengths can be designed to regulate the amount of protein produced from this transcript (Salis at al., 2009).

RBS strength scale and units: The output values range from 1 to 1000000 on a linear scale with arbitrary units that were experimentally validated using fluorescent protein abundance as a measure of expression strength (Salis at al., 2009). Designed sequences of a particular strength are not unique, i.e. different nucleotide sequences can encode an RBS having the same RBS strength. Furthermore, RBS strength is always context-dependent, therefore a designed sequence of a defined strength is only valid for the coding sequence it was calculated for. The minimum nucleotide sequences defining RBS strength are 35 base pairs upstream and 50 base pairs downstream of the start codon (ATG) of a coding sequence.

Transgene: a gene or genetic material that has been transferred naturally or by any of a number of genetic engineering techniques from one organism to another. The transgene that is transferred to the recipient can be from other individuals of the same species or even from unrelated species.

### DETAILED DESCRIPTION OF THE INVENTION

I: In a first aspect, the invention provides a composition for use as a medicament, comprising cells of a recombinant microorganism, wherein said microorganism comprises one or more transgenes or recombinant nucleic acid molecules encoding one of more proteins selected from:

(a) tryptophan 5-hydroxylase (EC 1.14.16.4),

(b) tryptophan decarboxylase (EC 4.1.1.28), and

(c) tryptamine 5-hydroxylase (EC:1.14.-.-),

The cells of said microorganism are therefore capable of producing increased amounts of one or more of 5-hydroxytryptophan (5-HT), 5-hydroxytryptamine (5-HTP) and tryptamine (TRM) as compared to cells of the non-recombinant microorganism from which it was derived.

In a further aspect thereof, said one or more recombinant nucleic acid molecules encode proteins selected from the group:

(a) tryptophan 5-hydroxylase (EC 1.14.16.4),

(b) tryptophan decarboxylase (EC 4.1.1.28),

(c) tryptamine 5-hydroxylase (EC:1.14.-.-),

(d) tryptophan 5-hydroxylase (EC 1.14.16.4) and tryptophan decarboxylase (EC 4.1.1.28), and

(e) tryptophan decarboxylase (EC 4.1.1.28) and tryptamine 5-hydroxylase (EC:1.14.-.-).

In a further aspect thereof, said composition is for use as a therapeutic in preventing and/or treating TRM-; 5-HTP-, or 5-HT-related disorders of the central nerve system (CNS); enteric nervous system (ENS); gastro intestine (GI); hormonal imbalance, metabolic disease, non-alcoholic fatty liver disease (NAFLD); non-alcoholic steatohepatitis (NASH), diabetes in a mammal.

More specifically, TRM-, 5-HTP or 5-HT homeostasis of the CNS treated by the composition of the invention include anxiety- and depression-related behavior, as well as memory-, cognition-, and psychiatric-disorders; in particular generalized anxiety disorder, phobia disorder, social anxiety disorder, panic disorder, obsessive-compulsive disorder, post-traumatic stress disorder, chronic stress disorder, separation and situational anxiety, age-related memory decline, and disorders in spatial memory formation, alertness, focus, learning and cognition, autism, migraine and immune-related disorders. TRM- or 5-HT related therapies of the GI that are treated by the composition of the invention include motility disorders, metabolic syndrome, obesity, weight-control, inflammation-associated sickness, inflammatory bowel disease, irritable bowel syndrome (IBS); celiac disease, diverticular disease, and colorectal cancer.

In another aspect the invention provides a composition for use as a medicament comprising a recombinant microorganism, wherein the microorganism comprises both one or more transgenes or recombinant nucleic acid molecules encoding:

(a) tryptophan 5-hydroxylase (EC 1.14.16.4), and tryptophan decarboxylase (EC 4.1.1.28), or

(b) tryptophan decarboxylase (EC 4.1.1.28) and tryptamine 5-hydroxylase (EC:1.14.-.-),

and additionally transgenes or recombinant nucleic acid molecules encoding:

(c) serotonin acetyltransferase (AANAT) EC 2.3.1.87 and

(d) acetylserotonin O-methyltransferase (ASMT) (EC 2.1.1.4),

wherein the cells are capable of producing melatonin. The cells are therefore capable of producing melatonin in amounts that are increased as compared to cells of the non-recombinant microorganism from which it was derived.

In a further aspect thereof, the invention provides a composition for use as a medicament, wherein said composition comprises cells of a recombinant microorganism capable of producing melatonin; suitable for oral delivery in a patient suffering from a melatonin-deficient or melatonin-related disorders, such as the melatonin-related disorders: circadian rhythm disturbance, insomnia, jet lag, autism; dementia, mood disorders, severe pain, cancer, and diabetes type 2.

In a further aspect thereof, said composition is for oral administration to a mammal in need thereof.

In a further aspect thereof, the microorganism in said composition is a live facultative anaerobic gut bacterium or a yeast (Ianiro G et al., 2014), preferably a commensal or probiotic strain, characterized by the ability to survive and/colonize one or more regions of the mammalian gut. For example the bacterium is one selected from the genus Escherichia, Bacteroides, Clostridium, Feacalibacterium, Eubacterium, Ruminococcus, Peptococcus, Peptostreptococcus, Lactobacillus, Lactococcus, Bifidobacterium, Enterococcus, Streptococcus, Pediococcus, Leuconostoc, Staphylococcus and Bacillus. Suitable species of bacterium include Escherichia coli (e.g. strain Nissle), Lactobacillus plantarum, Lactobacillus acidophilus, Lactobacillus casei, Lactobacillus reuteri, Lactobacillus rhamnosus, Lactococcus lactis, Bifidobacterium longum and Bifidobacterium adolescentis. Additionally, a suitable microorganism in said composition may be selected from microbial species identified by Li et al., 2014, and Zou et al., 2019, whose teaching is incorporated by reference.

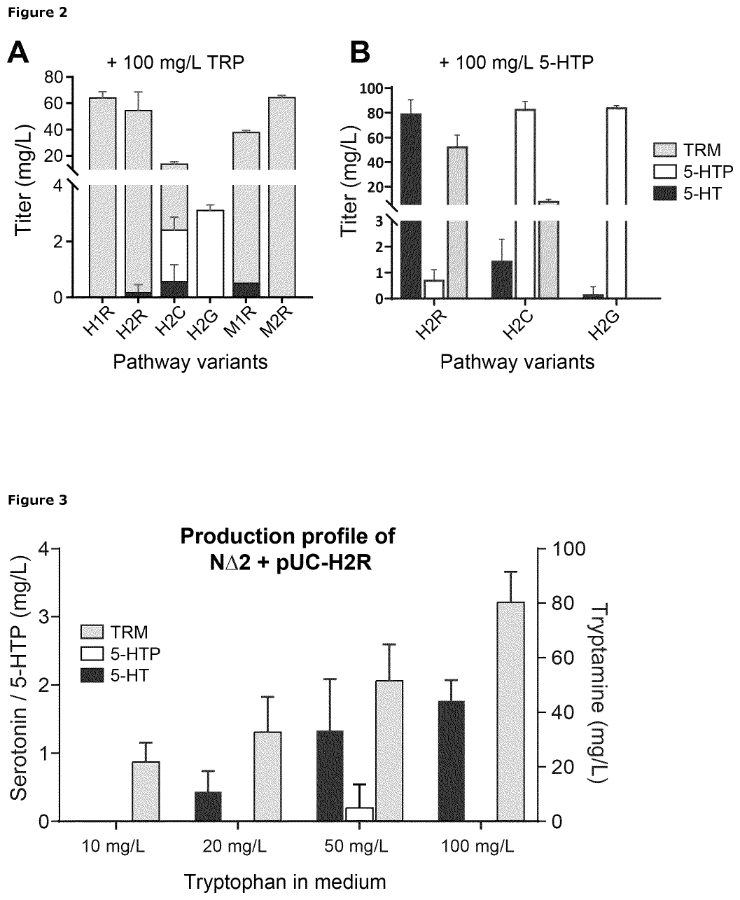

In a further aspect thereof, the microorganism in said composition comprises a nucleic acid sequence encoding tryptophan 5-hydroxylase (TPH) (EC 1.14.16.4), which catalyzes the conversion of L-tryptophan+tetrahydrobiopterin+O(2)=>5-hydroxy-L-tryptophan+4a-hydroxytetrahydrobiopterin by hydroxylating tryptophan at the C5 position (FIG. 1). The assay and quantification of 5-hydroxy-L-tryptophan yield are described in 1.1.2 and 1.1.3, respectively. Following oral administration to a mammal, the microorganism in said composition, is capable of expressing the TPH enzyme in the mammalian gut such as to produce and secrete 5-HTP (FIG. 2A). Both of the isoforms of tryptophan hydroxylase, TPH1 and TPH2, when expressed in a microorganism of the invention are suitable for producing 5-HTP; as well as 5-HT when co-expressed with a TDC having EC 4.1.1.28 (FIG. 2A). However, in a preferred embodiment the expressed TPH enzyme in the microorganism in the composition, for administration to the mammalian gastrointestinal tract, is TPH1 due to the low oxygen environment of the gut and TPH1's higher oxygen binding affinity, as compared to TPH2.

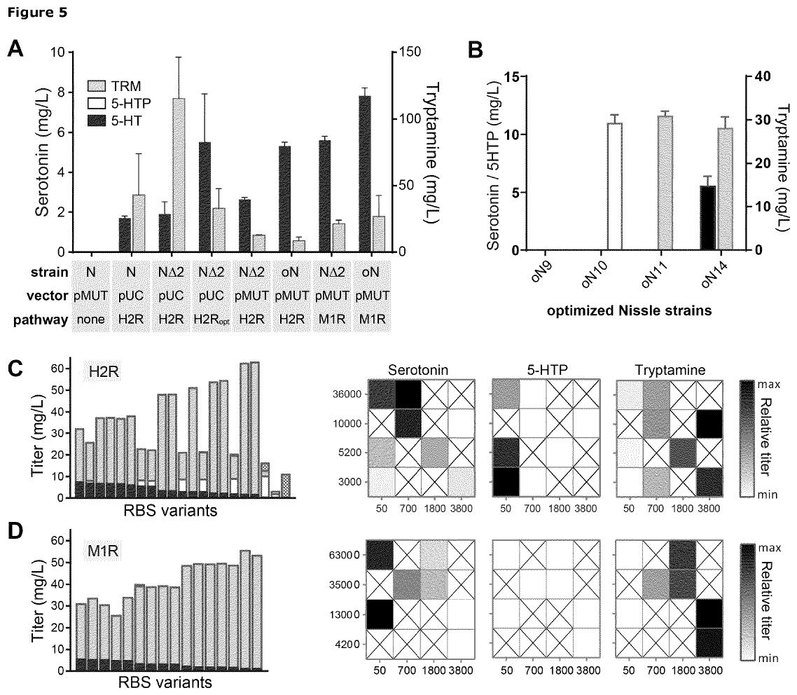

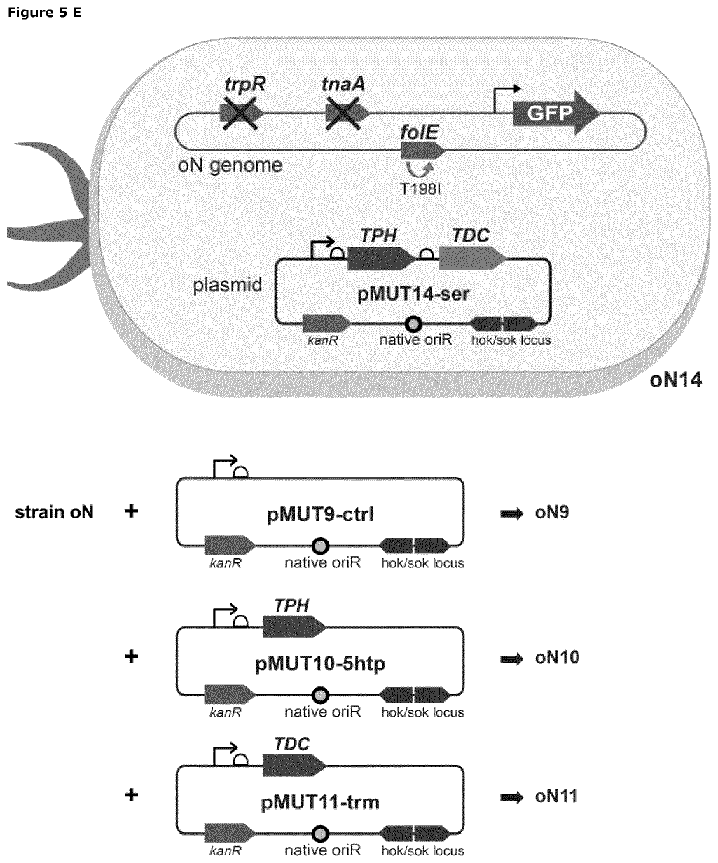

The microorganism in said composition may, either additionally or alternatively, comprise a nucleic acid molecule encoding tryptophan decarboxylase (TDC) (EC 4.1.1.28), which catalyzes the conversion of 5-hydroxy-L-tryptophan=>5-hydroxytryptamine+CO₂ (FIG. 1). The assay and quantification of 5-hydroxytryptamine yield is described in 1.1.2 and 1.1.3. A composition comprising cells of a microorganism capable of expressing both the TPH and TDC enzymes is able to produce and secrete 5-HT in the mammalian gut (FIG. 2A). In contrast, a composition comprising cells of a microorganism capable of expressing the TDC enzyme is able to produce and secrete TRM in the mammalian gut (FIGS. 1, 5B). The 5HT productivity of the microorganism can be modulated by the chosen combination of TPH and TDC enzymes expressed in the cells (Example 1). Accordingly, in some microorganisms the combination of expressed enzymes are (a) tryptophan 5-hydroxylase whose amino acid sequence has at least 80% sequence identity to SEQ ID No.: 6 or 12, and wherein the amino acid sequence of said tryptophan decarboxylase has at least 80% sequence identity to SEQ ID No.: 18.

In a further aspect, the microorganism in said composition comprises a recombinant nucleic acid molecule or transgene encoding tryptamine 5-hydroxylase, which catalyzes the conversion of O2+reduced [NADPH—hemoprotein reductase]+tryptamine=>H++H2O+oxidized [NADPH—hemoprotein reductase]+serotonin (FIG. 1). The assay and quantification of tryptamine yield is described in 1.1.2 and 1.1.3. Preferably, the microorganism in said composition further comprises a nucleic acid sequence encoding TDC. Following oral administration to a mammal, the microorganism in said composition, is capable of expressing the TPH and T5H enzymes in the mammalian gut such as to produce and secrete 5-HT (FIG. 1).

In a further aspect, the microorganism in said composition comprises nucleic acid molecules or transgenes encoding either TPH and TDC, or TDC and T5H,

as well as the enzymes:
serotonin acetyltransferase (AANAT) EC 2.3.1.87, which catalyzes the conversion of acetyl-CoA and serotonin to CoA and N-acetyl-serotonin and
acetylserotonin O-methyltransferase (ASMT) (EC 2.1.1.4), catalyzes the last reaction in the production of melatonin from L-tryptophan, the conversion of N-acetyl-serotonin and S-adenosyl-L-methionine (SAM) to melatonin and S-adenosyl-L-homocysteine (SAH). SAH can then be recycled back to SAM via the S-adenosyl-L-methionine cycle in microbial cells where the S-adenosyl-L-methionine cycle is native (or exogenously added) and constitutively expressed, such as, e.g., in E. coli (FIG. 1).

In a further aspect, the microorganism is genetically modified by the introduction of heterologous nucleic acid molecules encoding said 5-HT or melatonin pathway enzymes, including TPH, TDC, T5H, AANAT and ASMT enzymes; where the heterologous nucleic acid molecules are each cognately linked to a promoter, such as a constitutive or inducible promoter. The heterologous nucleic acid molecules encoding said enzymes may be cloned within an operon linked to a common cognate promoter. The heterologous nucleic acid molecules may be cloned into a self-replicating episome introduced into the microorganism, or may be cloned into the chromosome of the microorganism. The episome may be a native plasmid of the microorganism or a heterologous plasmid. Preferably, said plasmid is devoid of genetic elements that facilitate transduction to another microbial cell; or alternatively the plasmid comprises genes encoding proteins essential for survival of said microbial cell (i.e. essential gene as described in Example 3).

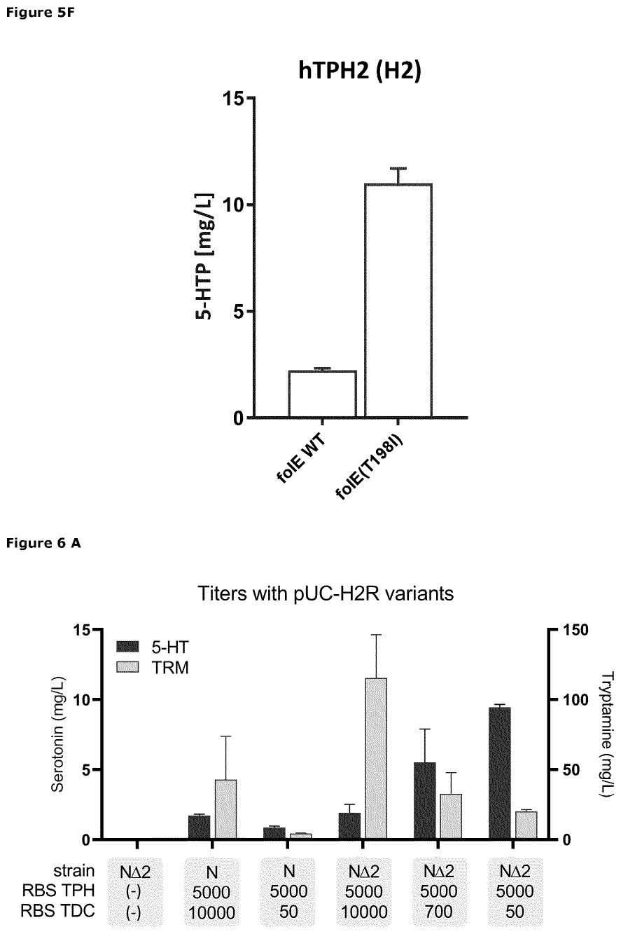

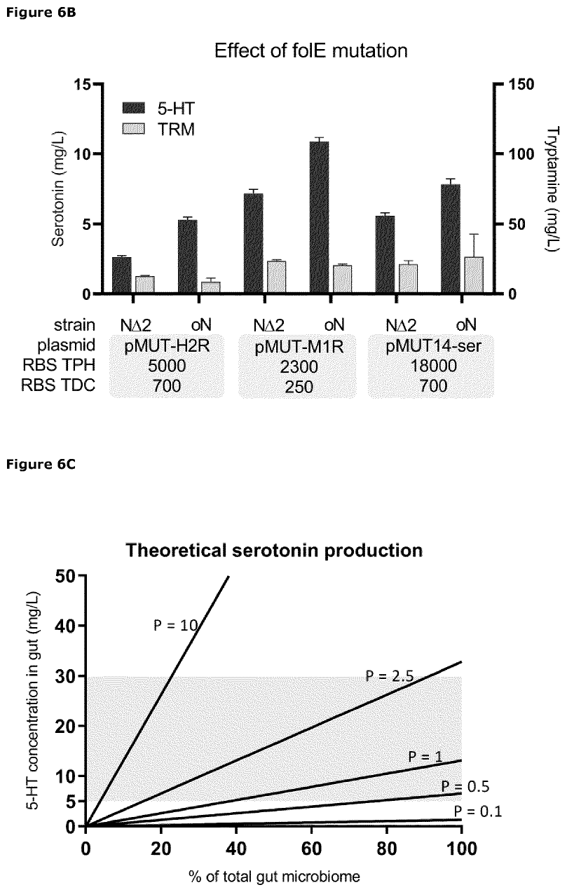

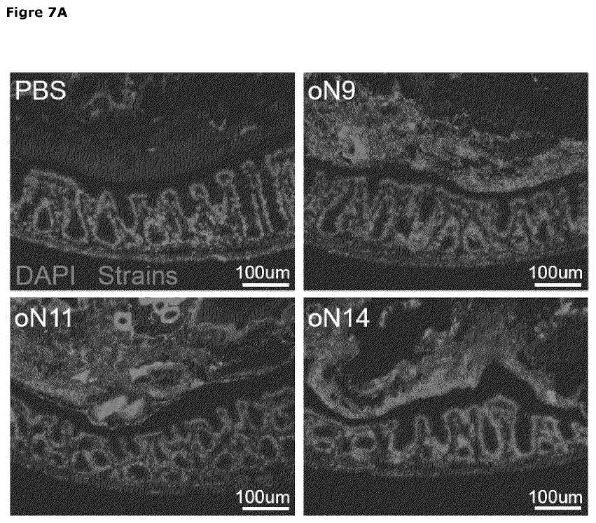

In a further aspect thereof, the microorganism is further genetically modified, in being devoid of genes capable of expressing a functional Trp operon repressor protein, and/or a functional tryptophanase (EC:4.1.99.1) (FIG. 1). A microorganism that is unable to express a functional Trp operon repressor protein, lacks the repressor protein needed to form a complex with L-tryptophan and bind the operator region of the trp operon (for example 5′-ACTAGT-‘3’) and is thus unable to prevent the initiation of transcription of the tryptophan biosynthetic pathway. A microorganism that is unable to express a functional tryptophanase (EC 4.1.99.1) is unable to catalyze the reaction: L-tryptophan+H(2)O<=>indole+pyruvate+NH(3). A microorganism of the invention devoid of genes capable of expressing a functional Trp operon repressor protein as well as a functional tryptophanase (EC 4.1.99.1) produces enhanced levels of 5-HT and TRM (Examples 2, 4 and FIGS. 3, 6A) due to enhanced flux into the tryptophan pathway.

In a further aspect thereof, the microorganism is further genetically modified, to comprise a gene encoding a mutant GTP cyclohydrolase I EC 3.5.4.16 (GCHI), wherein the mutant provides for an increased hydroxylation activity of said TPH, by at least 2-fold, or 3-fold, or 4-fold, or 5-fold, 6-fold or, 7-fold or, 8-fold or, 9-fold or, 10-fold, as compared to a native non-mutant GCH1 (e.g. parent GCH1), as measured by 5HTP yield. GCH1 catalyzes the regeneration of the tetrahydrobiopterin cofactor from GTP, required by TPH synthesis (FIG. 1). A microorganism of the invention expressing a mutant GCH1 enzyme, that is devoid of both trpR and tnaA genes, produces increased amounts of 5-HT and TRM (Example 4, FIG. 5A, 6A) due to enhanced flux towards 5HTP synthesis.

In a further aspect thereof, the microorganism comprises recombinant nucleic acid sequences encoding (a) tryptophan 5-hydroxylase (EC 1.14.16.4) and (b) tryptophan decarboxylase (EC:4.1.1.28), and is further genetically modified to regulate the relative expression levels of the TPH and TDC enzymes of the 5-HT pathway, such as to further increase the amounts of 5-HT produced, while reducing TRM production. By way of example, the expression of TPH and TDC is independently regulated by means of the RBS functionally linked to each of their respective nucleic acid coding sequences (i.e. genes). 5-HT yields are increased and TRM levels reduced, when the RBS of the TPH gene has a strength that exceeds the RBS of the TDC gene (measured as the relative strength of translation initiation), as shown in Example 4 (FIG. 5C, D; FIG. 6A). The 5-HT productivity of a microorganism of the invention, devoid of trpR and tnaA genes, is synergistically increased when the translation rate of TPH gene transcript exceeds that of the TDC gene transcript, by virtue of their respective functionally linked RBS (FIG. 6A, B).

The composition according to the present invention is for use as a medicament for animals, in particular mammals selected from the group consisting of humans, dogs, cats, pigs, cattle, horses, goats, and sheep, as well as poultry (e.g. chickens). In a one embodiment, the composition is for use as a medicament for pregnant or lactating women.

In therapeutic applications, such as treating TRM; 5-HTP-, or 5-HT-related disorders of the central nerve system (CNS); enteric nervous system (ENS); gastro intestine (GI) and metabolism or melatonin-related disorders in a mammal, the composition according to the invention is for administration in an amount sufficient to at least partially cure or arrest the symptoms of the disease and its complications. An amount adequate to accomplish this is defined as “a therapeutically effective dose”. Amounts effective for this purpose will depend on a number of factors known to those skilled in the art such as the severity of the disease and the weight and general state of the patient.

In prophylactic applications, similarly, the composition according to the invention is for administration to a subject, susceptible to or otherwise at risk of a particular disease, in an amount that is sufficient to at least partially reduce the risk of developing a disease. Such an amount is defined to be “a prophylactic effective dose”. Again, the precise amounts depend on a number of patient specific factors such as a patient's state of health and weight.

The microorganism of the invention can be provided as a pure form or can be incorporated in a matrix. Such matrix can advantageously protect the microorganism during the passage through the gastrointestinal tract (including the acidic conditions of the stomach) and enable live cells of the microorganism to arrive to the gut. Such protective matrix can comprise sugar(s) (such as maltodextrin), proteins or fat component. In one embodiment the protective matrix comprises or is a vegetable oil. The microorganism may be cultured according to any suitable method and prepared for encapsulation or addition to a nutritional composition by freeze-drying or spray-drying for example. Alternatively, it may be purchased already prepared in a suitable form for addition to food products.

A suitable nutritional composition includes a dairy product, a beverage powder, a dehydrated soup, a dietary supplement, a meal replacement, a nutritional bar, a cereal, a confectionery product, animal feed supplement or a dry pet food.

The composition of the present invention may further contain protective hydrocolloids (such as gums, proteins, modified starches), binders, film forming agents, encapsulating agents/materials, wall/shell materials, matrix compounds, coatings, emulsifiers, surface active agents, solubilizing agents (oils, fats, waxes, lecithins etc.), adsorbents, carriers, fillers, co-compounds, dispersing agents, wetting agents, processing aids (solvents), flowing agents, taste masking agents, weighting agents, jellifying agents, gel forming agents, antioxidants and antimicrobials. The composition may also contain conventional pharmaceutical additives and adjuvants, excipients and diluents, including, but not limited to, water, gelatine of any origin, vegetable gums, ligninsulfonate, talc, sugars, starch, gum arabic, vegetable oils, polyalkylene glycols, flavouring agents, preservatives, stabilizers, emulsifying agents, buffers, lubricants, colorants, wetting agents, fillers, and the like. In all cases, such further components will be selected having regard to their suitability for the intended recipient.

In a preferred aspect of the present invention, the composition further contains at least one prebiotic. Prebiotics can thus promote colonization of microorganism of the invention, in the intestines of a subject following oral administration and thereby enhance the effect of the microorganism contained in the composition according to the invention. Furthermore, several prebiotics have a positive influence on, e.g., digestion.

Preferably the prebiotic may be selected from the group consisting of oligosaccharides and optionally contain fructose, galactose, mannose, soy and/or inulin; dietary fibers; or mixtures thereof. The composition of the present invention may be provided in powder form having a water activity less than 0.2, for example in the range of 0.19-0.05, preferably less than 0.15.

Following oral administration, the microorganism of the invention is capable of surviving and/or colonizing one or more regions of the gut, including the duodenum, small intestine, cecum, proximal and distal colon. As exemplified in Example 6, the orally administered microorganism colonized primarily the cecum, proximal and distal colon.

Following colonization of the gut, the microorganism of the invention is shown to produce 5-HT, detectable in feces thereof, and importantly, to be accompanied by increased levels of 5-HT in the mucosal layers of the gut (Example 6). Furthermore, the colonizing microorganism is seen to induce therapeutically beneficial changes in both gene expression and gut physiology. In particular, observed changes in gut physiology include an increased mucosal layer, the maintenance of levels of markers of gut barrier function, and decreased levels of markers for gut inflammation, each of which is commensurate with a therapeutic effect on 5-HT related GI disorders. The expression of the 5-HT related receptors, HTR1B and HTR1D, in the gut was also observed, following administration of a microorganism of the invention producing elevated levels of 5-HT or TRM commensurate with a therapeutic effect on 5-HT related CNS disorders, including migraines (Example 8).

Administration of a composition according to the invention to a subject is demonstrated to have a prophylactic and therapeutic effect on a CNS related disorder from the pre-clinical trials preformed herein, using the well-established methods: Forced Swim Test (FST) and the Open Field Test (OFT) (Example 9 and 10).

II. In a second aspect, the invention provides a recombinant bacterium, comprising one or more recombinant nucleic acid molecules or transgenes encoding one of more proteins selected from:

(a) tryptophan 5-hydroxylase (EC 1.14.16.4),

(b) tryptophan decarboxylase (EC 4.1.1.28), and

(c) tryptamine 5-hydroxylase (EC:1.14.-.-)

wherein said microorganism is devoid of genes capable of expressing

trp operon repressor protein and tryptophanase (EC:4.1.99.1), and

wherein the cell is capable of producing increased amounts of one or more of 5-hydroxytryptophan, 5-hydroxytryptamine and tryptamine as compared to a cell of the non-recombinant microorganism from which it was derived.

In a further aspect thereof, said one or more recombinant nucleic acid molecules or transgenes encode proteins selected from the group:

(a) tryptophan 5-hydroxylase (EC 1.14.16.4),

(b) tryptophan decarboxylase (EC 4.1.1.28),

(c) tryptamine 5-hydroxylase (EC:1.14.-.-),

(d) tryptophan 5-hydroxylase (EC 1.14.16.4) and tryptophan decarboxylase (EC 4.1.1.28), and

(e) tryptophan decarboxylase (EC 4.1.1.28) and tryptamine 5-hydroxylase (EC:1.14.-.-).

In a further aspect said bacterium comprises recombinant nucleic acid molecules encoding:

(a) tryptophan 5-hydroxylase (EC 1.14.16.4), and tryptophan decarboxylase (EC 4.1.1.28), or

(b) tryptophan decarboxylase (EC 4.1.1.28) and tryptamine 5-hydroxylase (EC:1.14.-.-), and

additionally recombinant nucleic acid molecules encoding:

(c) serotonin acetyltransferase (AANAT) EC 2.3.1.87 and

(d) acetylserotonin O-methyltransferase (ASMT) (EC 2.1.1.4),

wherein the cells are capable of producing melatonin, in amounts that are increased as compared to cells of the non-recombinant bacterium from which it was derived.

In a further aspect thereof, the bacterium further comprises a gene encoding a mutant GTP cyclohydrolase I (GCHI), whose amino acid sequence has at least 70, 71, 73, 74, 75, 76, 77, 78, 79, 80, 81, 82, 83, 84, 85, 86, 87, 88, 89, 90, 91, 92, 93, 94, 95, 96, 97, 98 or 99% sequence identity to a wild-type GCHI having SEQ ID NO: 2, and comprises one or more mutations, wherein the mutant provides for an increased hydroxylation activity of said TPH, as compared to a native GCH1.

By way of example, the mutant GTP cyclohydrolase I (GCH1) is one having at least one or more mutations in an amino acid residue selected from the group consisting of D97, M99, T101, V102, A125, K129, N170, V179, T196, T198, S199, L200, S207, H212, E213, F214, L215 and H221, wherein the mutation in N170 is N170K, N170D or N170L; the mutation in V179 is V179A; the mutation in H212 is H212R or H212K; and the mutation in H221 is H221R or H221K. Preferably the mutation is a substitution selected from T198I, T198S, F214S, V179A, M99I and L200P.

The skilled person would be able to test for further mutants by co-expression of the mutant GCH1 and tryptophan 5-hydroxylase in a microbial cell and testing for increased activity of tryptophan hydroxylation to 5-hydroxytryptophan. Preferably, the mutant GCH1 increases the hydroxylation activity of tryptophan 5-hydroxylase by at least 2-fold, or 3-fold, or 4-fold, or 5-fold, 6-fold or, 7-fold or, 8-fold or, 9-fold or, 10-fold, as compared to the non-mutant GCH1 from which the mutant was derived, as measured by 5-hydroxytryptophan yield.

In a further aspect thereof, said bacterium is capable of expressing a protein having tryptophan 5-hydroxylase (EC 1.14.16.4) activity, where the amino acid sequence of said protein has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 6, 8, or 12.

In a further aspect thereof, said bacterium is capable of expressing a protein having tryptophan decarboxylase (EC 4.1.1.28) activity, where the amino acid sequence of said protein has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 14 or 18.

In a further aspect thereof, said bacterium is capable of expressing a protein having tryptamine 5-hydroxylase (EC:1.14.-.-) activity, where the amino acid sequence of said protein has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 20.

In a further aspect thereof, said bacterium is capable of expressing a protein having serotonin acetyltransferase EC 2.3.1.87 activity, where the amino acid sequence of said protein has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 22.

In a further aspect thereof, said bacterium is capable of expressing a protein having acetylserotonin O-methyltransferase (EC 2.1.1.4),activity, where the amino acid sequence of said protein has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 24.

In a further aspect thereof, said bacterium comprising recombinant nucleic acid molecules encoding (a) tryptophan 5-hydroxylase (EC 1.14.16.4), and (b) tryptophan decarboxylase (EC 4.1.1.28), wherein the tryptophan 5-hydroxylase has at least 80% sequence identity to SEQ ID No.: 6 or 12, and wherein the amino acid sequence of said tryptophan decarboxylase has at least 80% sequence identity to SEQ ID No.: 18.

In a further aspect thereof, said bacterium comprising recombinant nucleic acid molecules encoding (a) tryptophan 5-hydroxylase (EC 1.14.16.4), and (b) tryptophan decarboxylase (EC 4.1.1.28), is further genetically modified to up-regulate the expression level of TPH relative to TDC enzymes in the 5-HT pathway, such as to further increase the amounts of 5-HT produced, and reduce TRM production. By way of example, the nucleic acid molecules encoding tryptophan 5-hydroxylase and tryptophan decarboxylase are each functionally linked to their respective RBS, where the ratio of their strengths is at least 1.5: 1.0; 1.75: 1.0; 2.0:1.0; 2.5:1.0; 3.0:1.0; 3.5:1.0; 4.0:1.0; 4.5:1.0; 5.0:1.0; 5.5:1.0; 6.0:1.0; 6.5:1.0; 7.0:1.0; 7.5:1.0; 8.0:1.0; 8.5:1.0; 9.0:1.0; 9.5:1.0; 10.0:1.0; 20:1; 40: 1; 60: 1.0; 80: 1.0; 100:1.0; 150:1.0; 200:1.0; 250:1.0; and 300:1.0 more preferably at least 20.0:1.0; where RBS strength is measured as defined above, with reference to Salis at al., 2009.

Alternatively, said bacterium comprising recombinant nucleic acid molecules encoding (a) tryptophan 5-hydroxylase (EC 1.14.16.4), and (b) tryptophan decarboxylase (EC 4.1.1.28), is further genetically modified to up-regulate the expression level of TDC relative to TPH enzymes in the 5-HT pathway, such as to further increase the amounts of TRM production. For this purpose, the nucleic acid molecules encoding tryptophan decarboxylase and tryptophan 5-hydroxylase are each functionally linked to their respective RBS, where the ratio of their strengths is at least 1.5: 1.0; 1.75: 1.0; 2.0:1.0; 2.5:1.0; 3.0:1.0; 3.5:1.0; 4.0:1.0; 4.5:1.0; 5.0:1.0; 5.5:1.0; 6.0:1.0; 6.5:1.0; 7.0:1.0; 7.5:1.0; 8.0:1.0; 8.5:1.0; 9.0:1.0; 9.5:1.0; 10.0:1.0; 20:1; 40:1; 60:1.0; 80: 1.0; 100:1.0; 150:1.0; 200:1.0; 250:1.0; and 300:1.0 more preferably at least 2.0:1.0;

In a further aspect thereof, one or more recombinant nucleic acid molecules encoding (a) tryptophan 5-hydroxylase (EC 1.14.16.4), and/or (b) tryptophan decarboxylase (EC 4.1.1.28), in the bacterium of the invention, is functionally linked to a promoter that regulates expression constitutively or is inducible. When the promoter is a constitutive promoter, a suitable promoter may be selected from among the synthetic promoters described in http://parts.igem.org/Promoters/Catalog/Anderson), preferably selecting a strong promoter having a measured strength of ≥0.5 on the Anderson scale.

In a further aspect thereof, said bacterium is not capable of expressing a functional trp operon repressor protein, where the amino acid sequence of said protein has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 26.

In a further aspect thereof, said bacterium is not capable of expressing a protein having tryptophanase activity (EC:4.1.99.1), where the amino acid sequence of said protein has at least 70, 71, 73, 74, 75, 76, 77, 78, 79, 80, 81, 82, 83, 84, 85, 86, 87, 88, 89, 90, 91, 92, 93, 94, 95, 96, 97, 98, 99 or 100% sequence identity to SEQ ID NO: 29.

In a further aspect thereof, said bacterium is not antibiotic resistant to one or more clinically-used antibiotic agents.

The recombinant bacterium according to the second aspect, in its various described forms, is suitable for use in a composition according to each of the further aspects of the invention.

### EXAMPLES

### Example 1

### Genetically Modified Escherichia coli Cells Express a Biosynthetic Pathway for 5-HT Production

A 5-HT biosynthetic pathway was introduced into cells of E. coli Nissle to establish a synthetic serotonin pathway to catalyze the two consecutive metabolic steps of converting TRP to 5-HTP and then 5-HTP to 5-HT (FIG. 1). Identification of the optimal combination enzymes to catalyze these two metabolic steps was determined by expressing and determining the 5-HT yield obtainable from the combination of TPH and TDC genes.

1.1 Methodology

1.1.1 Modified E. coli cells were engineered as follows, with reference to Table 1:

The host strain, E. coli Nissle 1917 (Mutaflor®) was purchased from Ardeypharm GmbH, Germany. The gene for green fluorescent protein (GFP, GenBank: CAH64882.1) was placed under control of a strong, constitutive promoter (Part BBa_J23101, Registry of Standard Biological Parts, www.parts.igem.org) and integrated into the E. coli Nissle genome at the Tn7 attachment site using pGRG25 as described by McKenzie et al., (2006). Genes encoding TPH proteins derived from mammalian or plant sources: Human TPH1 (H1); human THP2 (H2); mouse TPH1 (M1); mouse THP2 (M2), and genes encoding TDC were derived from rice TDC (R), Cataranthus roseus (C), guinea pig (G) and Candidatus Koribacter versatilis Ellin345 (ck) were synthesized employing codons optimized for E. coli using the online tool from IDT (idtdna.com/CodonOpt). The tested TPH and TDC gene combinations were cloned as an operon, under the control of a synthetic promoter BBa-J23107, in a self-replicating plasmid (using pUC as backbone), comprising a kanamycin resistance gene. The plasmids were subsequently transformed into the host strain, by employing standard cloning and transformation procedures known in the art.

1.1.2 Growth and in vitro metabolite production was determined as follows:

Strains were grown in a modified M9 medium, containing 1× M9 salts (M6030, Sigma Aldrich), 0.2% (w/v) glucose, 0.1% (w/v) casamino acids (Cat. No. C2000, Teknova), 1 mM MgSO4, 50 μM FeCl3, 0.2% (v/v) 2YT medium (composition see below), and 50 mg/L of L-tryptophan unless stated otherwise. Kanamycin was added at a final concentration of 50 mg/L unless stated otherwise. Three single colonies were picked from each strain, grown in 300 μl medium in 96 deep-well plates and shaken at 250 rpm at 37° C. for 16 hours. The main culture was inoculated by diluting this pre-culture 1:100 into fresh medium, and cells were grown for 24 h under the same conditions. In the case of substrate feeding, TRP and 5-HTP was added to the growth medium in an amount of 100 mg TRP or 5-HTP/L. Afterwards, the culture supernatant was separated from cells using a 0.2 μm pore size filter, and frozen at −20° C. until analysis by LC-MS. All data shown are mean+/−SD from at least 3 biological replicates.

1.1.3 Metabolite production was quantified by LC-MS as follows:

Detection of serotonin, tryptamine, tryptophan and 5-HTP were conducted by liquid chromatography mass spectrometry (LC-MS) measurements on a Dionex UltiMate 3000 UHPLC (Fisher Scientific, San Jose, Calif.) connected to an Orbitrap Fusion Mass Spectrometer (Thermo Fisher Scientific, San Jose, Calif.). The system used an Agilent Zorbax Eclipse Plus C18 2.1×100 mm, 1.8 μm column kept at 35° C. The flow rate was 0.350 mL/min with 0.1% formic acid (A) and 0.1% formic acid in acetonitrile (B) as mobile phase. The gradient started as 5% B and followed a linear gradient to 35% B over 1.5 min. This solvent composition was held for 3.5 min after which it was changed immediately to 95% B and held for 1 min. Finally, the gradient was changed to 5% B until 6 min. The sample (1 uL) was passed on to the MS equipped with a heated electrospray ionization source (HESI) in positive-ion mode with sheath gas set to 60 (a.u.), aux gas to 20 (a.u.) and sweep gas to 2 (a.u.). The cone and probe temperature were 380° C. and 380° C., respectively, and spray voltage was 3500 V. Scan range was 50 to 500 Da and time between scans was 100 ms. Detection of serotonin (160.07646 ion), tryptamine (144.08158 and 161.10775 ions) and tryptophan (205.09785 ion) was conducted in full scan whereas the 5-HTP detection was carried out after HCD fragmentation (221.09>162.05553, 25% HCD CE). Quantification of the compounds was based on calculations from calibration standards analyzed before and after sets of 24 samples. All reagents used were of analytical grade.

1.2 Results:

Genetically modified strains of the host, E. coli Nissle, engineered to express the mouse TPH1 (M1) or human TPH2 (H2), in combination with TDC from rice (R) or Cataranthus roseus (C) were found to produce measurable amounts of serotonin production (FIG. 2A). When the strains were fed with the substrate 5-HTP during cultivation, cells expressing a gene encoding the rice TDC (R) produced the highest yields of 5-HT (79.5±11.1% yield), demonstrating that rice TDC was the most efficient in catalyzing the 5-HTP decarboxylation step (FIG. 2B). Accordingly, E. coli Nissle, expressing the ‘M1R’ and ‘H2R’ pathways, being 5-HT producers were selected for further optimization.

Feeding to TDC showed that all enzymes produced some serotonin, but the ‘R’ enzyme from rice had the highest conversion (right panel).

### Example 2

### 5-HT and TRM Production by Genetically Modified Escherichia coli Cells of the Invention is Enhanced by Increasing Tryptophan Pathway Flux

Increased availability of tryptophan pathway substrate, tryptophan, is shown to increase 5-HT production in 5-HT producing strain of the invention (FIG. 3). increase In light of this observation, the endogenous pools of available TRP were increased in genetically engineered cells of the invention were generated by inactivating endogenous genes encoding the tryptophan repressor trpR and tryptophanase tnaA, such as to enhance tryptophan pathway flux.

2.1 Methodology:

Knockouts of trpR and tnaA from the host E. coli Nissle genome were performed using CRISPR/Cas9 as described in (Mehrer et al., 2018). Briefly, a two-plasmid system consisting of an inducible cas9/λ-Red expression plasmid and a guide RNA (gRNA) plasmid were used to introduce double-strand breaks at the desired gene loci in the host genome. gRNAs were designed using CRISPy-web (Blin et al., 2016) after uploading the EcN genome sequence (GenBank: CP007799.1). Templates for homologous recombination at the selected cut site were generated as follows: where available, strains with the desired knockouts (ΔtrpR::FRT-kan-FRT or ΔtnaA::FRT-kan-FRT, respectively) from the KEIO collection (Baba et al., 2006) were transformed with pSIJ8 (Jensen et al., 2015) and the FLP recombinase gene was induced to remove the kanamycin resistance gene. Resulting colonies were screened for kanamycin sensitivity. The ΔtrpR::FRT or ΔtnaA::FRT loci were amplified using oligos binding 500 bp up and downstream of the FRT site to generate PCR products of approximately 1 kb. CRISPR/Cas9 and gRNA expression plasmids were cured from the strains as described previously (Mehrer et al., 2018).

Growth of host cells; their metabolite production and quantification was performed as described in example 1.1.2 and 1.1.3.

2.2 Results:

In order to direct a corresponding flux of endogenous tryptophan towards the 5-HT pathway, the strains of the invention were further engineered by knockouts of genes encoding trpR and tnaA from the host genome to generate host E. coli Nissle strains designated NΔ2. An exogenous supply of tryptophan is shown to further enhance 5HT and TRM production by such genetically modified E. coli Nissle cells of the invention (strain EcNΔ2+pUC-H2R in FIG. 3).

### Example 3

### Genetically Modified Escherichia coli Strains of the Invention Exhibit Stable 5-HT Production

When the 5-HT operon, encoding the 5-HT biosynthetic pathway, is cloned into a multi-copy plasmid in host cells of the invention, the production of 5-HT is dependent on the stability of the plasmids. Stability is preferably not dependent on plasmid genes conferring antibiotic resistance. Two solutions for conferring plasmid stability were compared; based on the plasmid backbone of pUC (as in example 1 and 2) and one of the two native plasmids in E. coli Nissle, pMUT1 (Blum-Oehler et al., 2003).

3.1 Methodology:

The high copy number 5-HT production plasmid (pUC-H2R) was modified by introducing a synthetic copy of the essential bacterial gene infA down stream of H2R pathway operon. The corresponding infA gene in the host genome was knocked out using the protocol described in Example 2.1, so as to generate E. coli Nissle strain NΔ3 (ΔtrpR, ΔtnaA, ΔinfA).

The operon comprising the H2R pathway was also cloned into plasmid pMUT1 (GenBank: A84793.1) as follows. The pMUT1 plasmid was isolated from wild-type E. coli Nissle, and amplified to serve as the pMUT backbone. Elements that could enable plasmid transfer were removed to generate pMUT (Δ bp 1-1323 and 1664-3117 from circular sequence A84793.1), and a kanamycin resistance gene and the H2R or M1R operons (without infA) were inserted into the backbone to generate pMUT-H2R or pMUT-M1R, respectively.

The following additional versions of the plasmids, pMUT-H2R or pMUT-M1R, were generated: the hok/sok toxin-antitoxin plasmid stability element (GenBank: MK134376, Region 58002-58601) was inserted into the pMUT-M1R backbone to generate the plasmid pMUT14-ser. The TPH gene was removed from pMUT14-ser to generate pMUT11-trm, and both TPH and TDC were removed to make pMUT09-ctrl.

Cell growth determination: Cells were grown as described in example 1.1.2, but wherein after inoculation of the main culture, 200 μl were transferred into a flat-bottom, clear 96-well microtiter plate; then sealed with a gas-permeable membrane (Z380059, Sigma Aldrich). Cultures were grown at 37° C. with 700 rpm orbital shaking, and their growth was recorded on a BioTek™ ELx800 plate reader at OD_{630 nm} every 10 minutes.

Plasmid stability determination: six single colonies each were picked from each tested strain were grown in 300 μl 2YT medium (containing 1.6% (w/v) tryptone, 1% (w/v) yeast extract, 0.5% (w/v) NaCl) without kanamycin in 96 deep-well plates and shaken at 250 rpm at 37° C. for 24 hours. The optical density (OD630) of the cultures was measured and cultures were diluted 1:1000 into fresh medium, and cells were plated on LB-Agar plates with and without kanamycin daily, corresponding to approximately 10 doublings every 24 h. Colony forming units on LB Agar plates+/−kanamycin were counted daily. At time points t=0, 50 and 100 generations, cultures were diluted 1:100 into the modified M9 medium described above (without kanamycin) and serotonin production was measured as described.

3.2 Results:

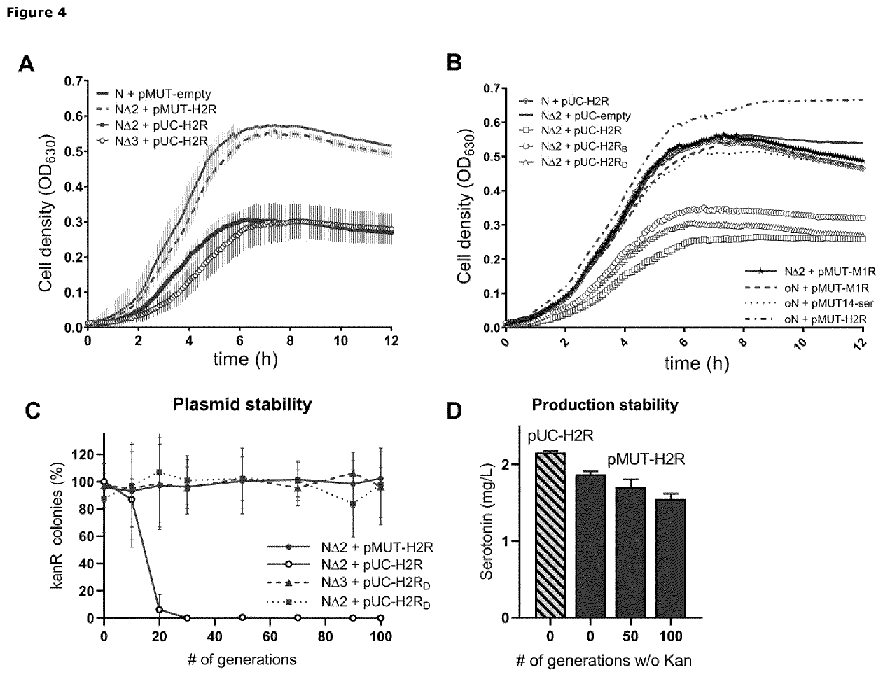

While stable, the pUC based plasmid expressing both the H2R pathway and infA genes caused an undesired growth defect in both NΔ2 and NΔ3 host cells (EcN with ΔtrpR, ΔtnaA, ΔinfA), likely due to metabolic burden (FIGS. 4A, B and C).

By contrast, the pMUT based plasmid (pMUT-H2R) was both stably maintained in NΔ2 host cells (FIG. 4A, B) and showed no detectable fitness defect for at least 100 generations, in the absence of selection pressure, in the form of antibiotic resistance (FIG. 4A, B, C). Additionally, 5-HT production by EcNΔ2 pMUT-H2R cells was stable over this period and comparable to initial pUC-H2R titers (FIG. 4D).

The hok/sok toxin-antitoxin module was introduced into the pMUT-M1R plasmids, producing pMUT14-ser and pMUT11-trm (Table 1) to further ensure their stability in vivo.

### Example 4

### Optimization of 5-HT Production by Genetically Modified Escherichia coli Strains of the Invention

Tryptamine (TRM) is a significant byproduct detected among the metabolites produced by the genetically engineered E. coli strains of the invention expressing a plant TDC enzyme (FIGS. 2A, 3). A genetic modification, designed to enhance flux towards 5HTP synthesis, was to increase the activity of GTP glycohydrolase (GCH1) activity and thereby enhance regeneration of the tetrahydrobiopterin cofactor, required by TPH synthesis. Further genetic modifications designed to fine-tune the relative expression strength of TPH and TDC were tested in order to further enhance 5-HT production.

4.1 Methodology

The coding sequence of the E. coli N folE gene was mutated by first amplifying the folE gene in 2 parts, and then introducing the T198I encoded mutation in the oligos used for the overlap-extension PCR. The resulting dsDNA fragments were purified and co-transformed with the gRNA plasmid to generate markerless gene mutations of the folE gene in the genome of cells of EcN.

RBS strength libraries were designed using the ‘RBS calculator v1.1’ (Salis et al., 2009) choosing library sizes of 4-6 variants for each of the co-expressed genes encoding TPH and TDC (M1R and H2R), ranging in theoretical strength from 50 to 50000 arbitrary units. Mutations in the RBS were introduced upstream of each gene with oligos containing degenerate bases and using Gibson assembly (Gibson et al., 2009). Transformants were picked randomly and RBSs sequenced after testing the strains' metabolite production profiles.

4.2 Results

The native folE gene in host E. coli Nissle cells of the invention was mutated to encode a mutant GCH1 (T198I) in order to enhance regeneration of the tetrahydrobiopterin cofactor. The production of 5-HT is increased, in these Optimized Nissle (oN) strains expressing the mutant GCH1 enzyme in combination with the 5-HT biosynthesis operon (oN pMUT1H2R or oN pMUTM1R) as compared to strains lacking this additional modification (NΔ2 pMUT-H2R or NΔ2 pMUT-M1R respectively) as seen in FIG. 5A. The mutant GCH1 increases the hydroxylation of tryptophan to 5-hydroxytryptophan catalyzed by human tryptophan 5-hydroxylase in strains comprising pMUT10-5htp, by 4-fold (FIG. 5F).

oN strains, expressing the mutant GCH1 enzyme in combination with either the 5-HTP biosynthesis pathway (oN10=oN pMUTM1), or TRM pathway (oN11=oN pMUTR) produced 5-HTP and TRM as the only detected products of the pathway, respectively (FIG. 5B).

Modulating the relative expression strength of TPH and TDC using selected combinations of ribosome binding sites (RBS) for their respective gene is shown to enhance 5-HT production and reduce accumulation of TRM (FIG. 5C and D). Increased 5-HT production was obtained when the relative strength of the RBS functionally linked to the TPH gene exceeded that of the RBS functionally linked to the TDC gene.

The synergistic effect of the genetic modifications introduced into E. coli Nissle strains comprising genes of the 5-HT pathway, on their respective 5-HT productivity and 5-HT:TRM ratio is demonstrated in FIG. 6. Knockout of the trpR and tnaA genes in the E. coli genome conferred a synergistic effect on 5-HT yields and 5-HT:TRM ratio in an E. coli cell provided that the relative expression of the TPH gene exceeded that of the TDC gene, by virtue of the functionally linked RBS (FIG. 6A). Introduction of the folE genomic mutation (encoding a T198I substitution) further increased 5-HT yields and 5-HT:TRM ratio when combined with the foregoing mutations in the E. coli Nissle strains (FIG. 66).

### Example 5

### Oral Administration of a Genetically Modified Escherichia coli Strain of the Invention is Predicted to Produce Therapeutic 5-HT Levels in a Subject

Therapeutically effective levels of 5-HT in the gut are reported to lie in the range of 5-30 mg/L (Liu, Q. et al., 2008). The relative abundance of 5-HT producing bacteria required to produce 5-HT within the therapeutically effective range in mouse gut is predicted for bacteria having an in vivo 5-HT production rate of 0.1 to 10 mg/L*OD₆₃₀*h (FIG. 6C). This is based on underlying assumptions with respect to density of bacteria in gut predicted to range from 1E2-E11/g (Sender et al., 2016), depending on the location (Casteleyn et al., 2010), as shown below.

| | | | Cell density* | |
|---|---|---|---|---|
| Intestinal | Volume | | cells/g | |
| segment | cm³ | % | (ml) | cells total |
| Duodenum | 0.21 | 15.7 | 100 | 21 |
| Jejunum | 0.69 | 51.5 | 1000 | 690 |
| Ileum | 0.07 | 5.2 | 50000000 | 3500000 |
| Caecum | 0.16 | 11.9 | 1E+10 | 1600000000 |
| Colon-rectum | 0.21 | 15.7 | 1E+11 | 21000000000 |
| Sum | 1.34 | 100 | | 2.3E+10 |

*E. coli: 1 OD600 nm (in standard 1 cm gap length spectrophotometer) = 0.36 g/L = 8*E8 cells/ml

In vitro, specific productivity (P) can be measured as serotonin titer (in mg/L) per bacterial load (in OD_{630 nm}) per hour. Since the 5-HT depletion rate is unknown, it is set to be equal to productivity in order to determine physiologically relevant concentrations (in mg/L). The genetically modified E. coli strains of the invention (e.g. strain oN14) when administered are predicted to be capable of producing 5-HT in vivo at near physiologically relevant levels.

### Example 6

### Oral Administration of a Genetically Modified Escherichia coli Strain of the Invention Enhances 5-HT Production in the Mouse Gut

Oral delivery of the genetically modified E. coli Nissle strains of the invention and their localization to the gut and enhancement of 5-HT production was demonstrated in mice.

6.1 Methodology

6.1.1 Oral administration: Male mice (C57BL/6) supplied by Taconic Biosciences or Jackson Laboratories, from 6-8 weeks of age were group-housed on a 12-h light:dark cycle (lights on from 7:00-19:00) at constant temperature with ad libitum access to food and water in a Specific Pathogen Free (SPF) facility. Upon delivery, mice were given one week to adjust to new location, followed by a 7-day, 3-rotation microbiome normalization protocol. On day one, three, and five of the normalization protocol, mice were randomly mixed. On day 6, mice were mixed one last time and separated in their respective cohorts with 2-3 cages per cohort with 3-5 mice in each cage, exact numbers depending on experiment. On day seven, mice began day zero of the experimental protocols, being tagged for identification and housed in groups of 3-5 mice in bottle fed cages, set in designated racks, and opened only under a laminar flow hood. Fecal samples were collected at day zero of the experimental protocol. The mice were then orally gavaged every 24 hrs at the same time of day for 10 days with 200 μl of either sterile PBS or freshly grown 10⁹ cells of E. coli Nissle strains designated: oN14 (E. coli Nissle strain oN comprising plasmid pMUT14-ser); oN11 (E. coli Nissle strain oN comprising plasmid pMUT11-trm), and oN9 (E. coli Nissle strain oN comprising plasmid control plasmid pMUT9-ctrl), in sterile PBS (FIG. 5E). 24 hours after the last gavage, fecal pellets were collected and mice were either euthanized for serum, intestinal tissue, and intraluminal fecal collection or were used for motility or behavioral testing.

6.1.2 Gut localization of oN strains: was determined by plating digesta derived from different intestinal regions on LB agar growth media comprising 50 mg/L kanamycin and quantifying the number of GFP positive, kanamycin resistant (KanR) colonies.

6.1.3 Immunodetection in thin sections: Colon samples were collected and 5 mm sections were cut in the mid-colon and fixed in either a Methanol-Carnoy (60% Methanol, 30% Chloroform, 10% Acetic Acid) Solution or 4% Paraformaldehyde (PFA) in Phosphate Buffered Solution (PBS) for 24 hours. Fixed samples were then washed three times and stored in 70% ethanol. Samples were kept intact with digesta in the lumen, embedded in Paraffin and cut luminally at 4 μm and either prepped for Immunohistochemistry or subjected to Hematoxylin and Eosin staining. For Immunohistochemistry: PFA fixed slides were used for Serotonin (5-HT) antibody staining and Methacarn fixed samples were used for Muc2, Green Fluorescent Protein (GFP), and Chromogranin A (CgA) antibody staining. All slides were baked for 2 hours at 57° C., deparaffinized in xylene and an ethanol-water gradient, rehydrated, then treated with an antigen retrieval solution of 10 mM sodium citrate, pH 6.0 for 30 minutes at 90° C. Slides were blocked for 30 minutes at room temperature with 5% fetal bovine serum (FBS) in PBS and then incubated with primary antibodies at 4° C. overnight in 5% FBS in PBS. Slides were washed with PBS three times, incubated with secondary antibody for 2 hours at room temperature in PBS, washed three times in PBS, incubated with DAPI (4′,6-diamidino-2-phenylindole) at 10 μg/ml for 5 minutes, washed three times in PBS and mounted using glycerol. Slides stained with primary antibodies pre-conjugated with Alexa fluorophores did not undergo secondary antibody staining. Antibodies Used: Anti-GFP rabbit IgG with Alexa Fluor 594 Conjugate (Life Technologies A21312). Anti-Muc2 polyclonal rabbit (Santa Cruz Technologies sc-15334). Anti-Chromagranin A polyclonal rabbit (Abcam ab15160). Anti-Serotonin polyclonal goat (Abcam ab66047). Secondary Anti-Rabbit Alexa Fluor 568 (Thermo-Scientific Novex a11011). Secondary anti-goat IgG Alexa Fluor 488 (Abcam ab150077). All images were taken with Nikon Eclipse Ti2-E Inverted Microscope System, image conversion and cell size (particle) analysis performed using NIS-Elements, Advanced Research Software and ImageJ (FIJI) for Macbook version 2.0.0-rc-69/1.52i.

6.1.4 In vivo metabolite analysis. Serum, tissue samples, and fecal samples were analyzed for serotonin, 5-hydroxytryptophan, tryptamine, and tryptophan. For serum: blood samples were collected via the inferior vena cava post euthanasia, samples were mixed 1:1 with 0.9% NaCl, 0.2% Ascorbic acid. Samples were kept at room temperature for 10 minutes then placed on ice until centrifugation at 2000 g for 10 minutes at 4° C. Supernatant was saved for analysis and immediately frozen at −20° C. For tissue analysis: colon tissue was weighed and immediately frozen on liquid nitrogen after collection and cryofractured using stainless steel microvials (Biospec 2007) with a single 6.34 mm stainless steel bead (Biospec 11079635ss) beat for 30 s in BeadBeater (Biospec 112011). Tissue was then resuspended in 0.9% NaCl, 0.1% Ascorbic Acid, centrifuged at 2000 g for 10 minutes at 4° C. Supernatant was saved for analysis and immediately frozen at −20° C. For fecal samples: samples were weighed, placed in Corning Cryovials with 0.5 ml of 0.9% NaCl, 0.1% Ascorbic Acid with 200 ml Zirconia/Silica beads (Biospec 11079101z) and beat for 5 minutes in BeadBeater at 4° C. Samples were centrifuged at 2000 g for 10 minutes at 4° C. Supernatant was saved for analysis and immediately frozen at −20° C.

6.2 Results

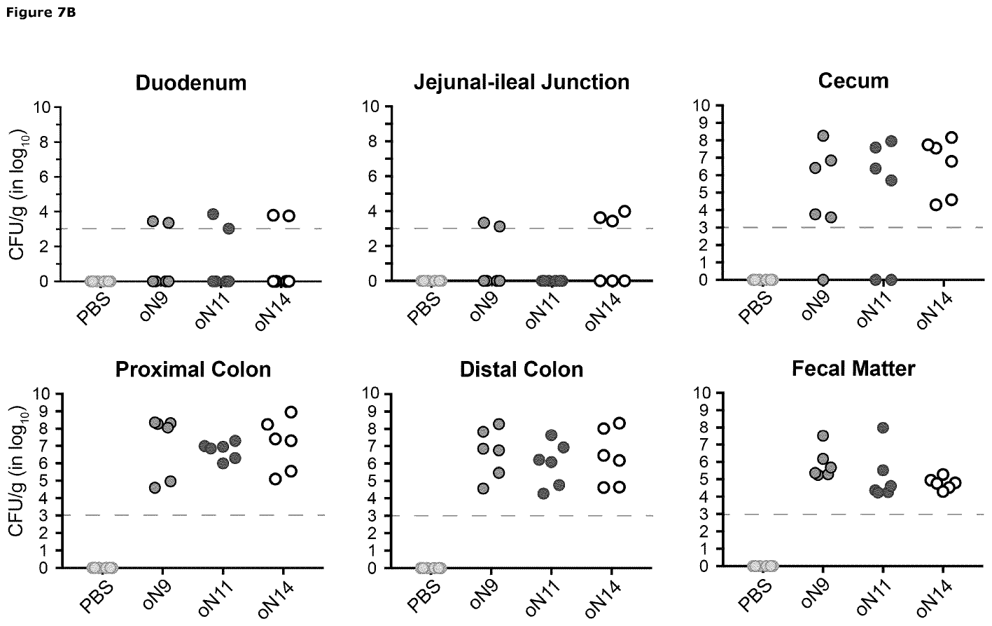

Following 10 days of oral administration of 10⁹ cells of E. coli Nissle strains oN14, oN11 and control strain oN9 (FIG. 5E) to mice, the administered cells were primarily found to accumulate in their large intestine. The highest concentration of oN cells was detected in the proximal colon with >10⁸ CFU/g (FIG. 7A), while fewest oN bacteria were detected in the small intestine with (<10⁴ CFUs/g). In the mid-colon, the oN cells were localized along the mucosal layer based on their visualization by immuno-histochemistry with an anti-GFP antibody (FIG. 7B).

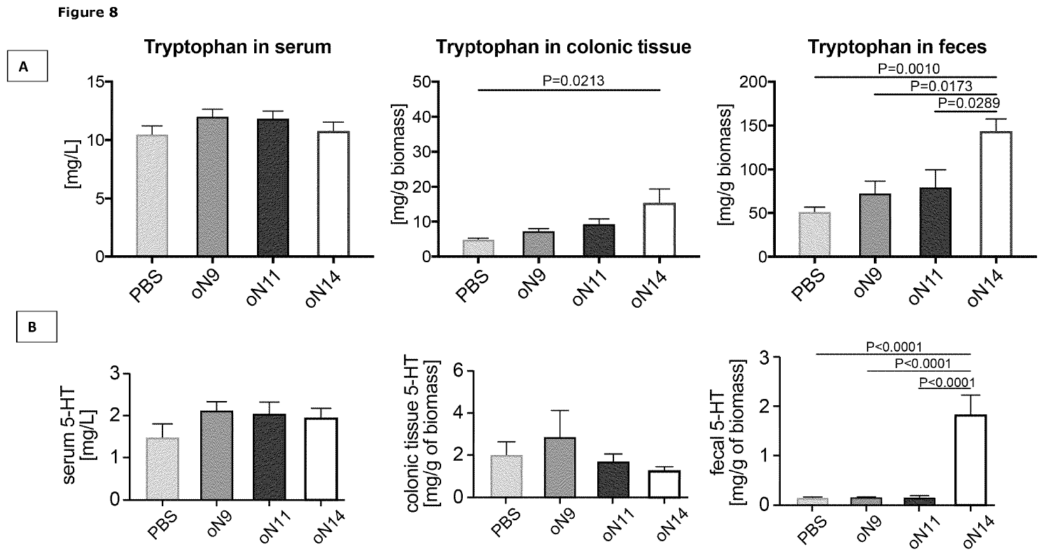

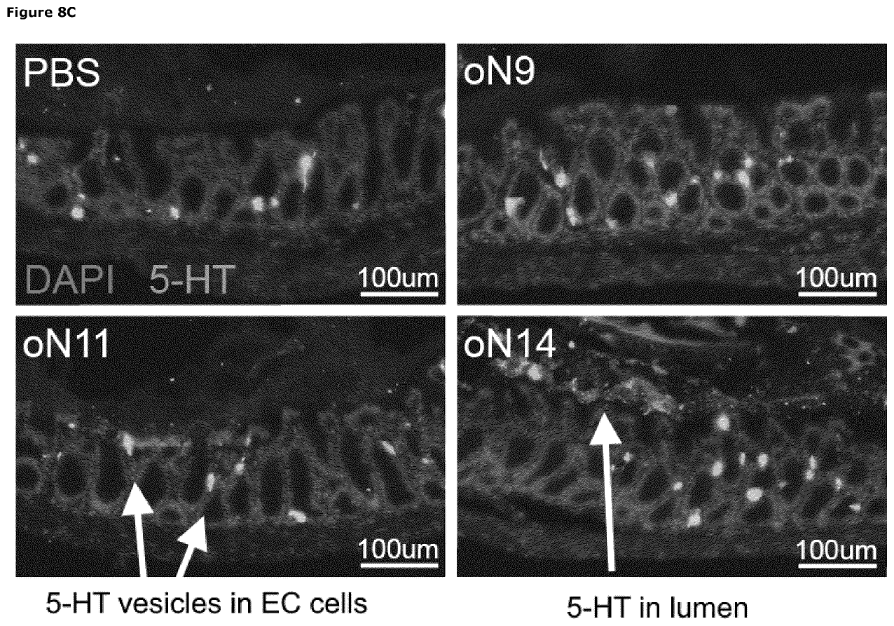

The level of TRP, 5-HTP, 5-HT and TRM were also measured in samples of serum, colonic tissue, and feces derived from the orally-treated mice. Tryptophan levels were elevated in colon tissues and feces, but not serum, and only in mice orally administered E. coli Nissle strain oN14 (FIG. 8A). 5-HT levels were not statistically different in serum and in colonic tissue across cohorts (p>0.4 by ANOVA). However, fecal 5-HT levels were significantly increased in mice administered E. coli Nissle strain oN14 (13.4-fold increase, p<0.0001 by ANOVA) compared to other groups (FIG. 8B). Tryptamine and 5-HTP levels were below the limit of detection in all samples.

Although 5-HT levels were not elevated in colonic tissues per se, (following washing to remove residual digesta), increased levels of serotonin were specifically detected along the mucosal layers of the colon in mice administered E. coli Nissle strain oN14 (FIG. 8C). These results demonstrate that orally administered cells of E. coli Nissle strain oN14 accumulate and produce 5-HT in vivo along the mucosa of the colon in mice.

### Example 7

### Oral Administration of a Genetically Modified Escherichia coli Strain of the Invention Elicits Changes in Gene Expression and Physiology in the Gut

Oral administration of E. coli Nissle strain oN14 to mice was shown to upregulate Muc2 expression and increase mucosal thickening, which in turn confers increased mucosal integrity. Excess serotonin derived from enterochromaffin cells can stimulate intestinal inflammation. However, even though increased levels of 5-HT were detected in mucosal layers of the colon of treated mice (Example 6), this did not lead to a change in expression of markers of intestinal inflammation, intestinal turnover or intestinal barrier function (not shown).

Host (mouse) TPH2 expression, but not TPH1, was increased in the colon of mice administered E. coli Nissle strain oN14 indicating that bacterially-derived 5-HT can induce TPH2-mediated neuronal 5-HT biosynthesis in the ENS of mice. The total number or size of EC cells (the primary producers of host 5-HT in the gut) in these mice was not however affected.

While tryptophan and serotonin metabolism can also affect the kynurenine pathway, whose dysfunction is linked to many neurologic and metabolic disorders, no changes were detected in the expression of genes encoding the rate-limiting enzymes that convert L-tryptophan to N-formylkynurenine, in the colon of mice administered E. coli Nissle strain oN14.

7.1 Methodology

7.1.1 Oral administration: as described in 6.1.1.

7.1.2 Gene expression analysis: Tissue cryofractured according to the protocol in Example 6.1.4. Tissue was then resuspended in Trizol (Invitrogen) according to the instructions of the manufacture for RNA isolation. Total RNA was resuspended in 30 μL of RNase-free water. RNA was converted to cDNA with iScript gDNA Clear cDNA synthesis Kit (BioRad 1725035) according to manufacture instructions. Quantitative Real Time polymerase chain reaction (qPCR) was performed in triplicates in 384 well-plates on Applied Biosystems QuantStudio6 Flex Real-Time PCR System according to manufacture instructions. Primers used are listed in Table 3. Cyclophilin was used as the housekeeping gene. Relative mRNA levels were quantified using the ΔΔCτ method.

7.1.3 Immunodetection in Thin Sections: as Described in Example 6.1.3.

7.2 Results

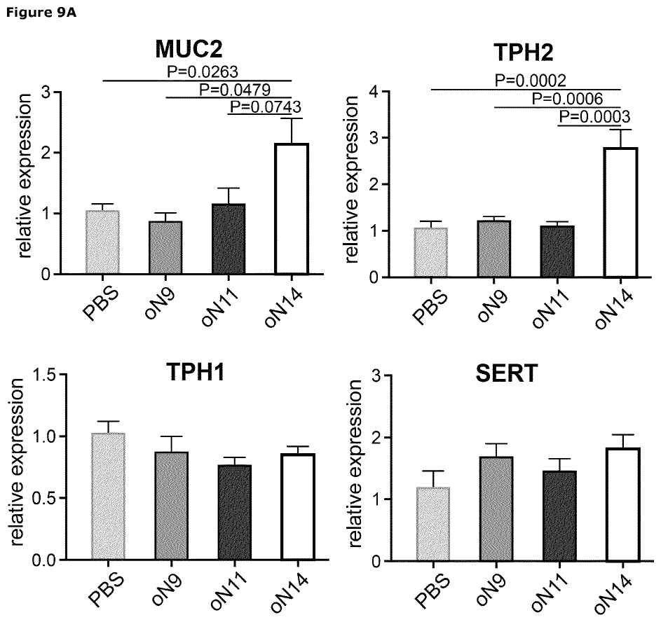

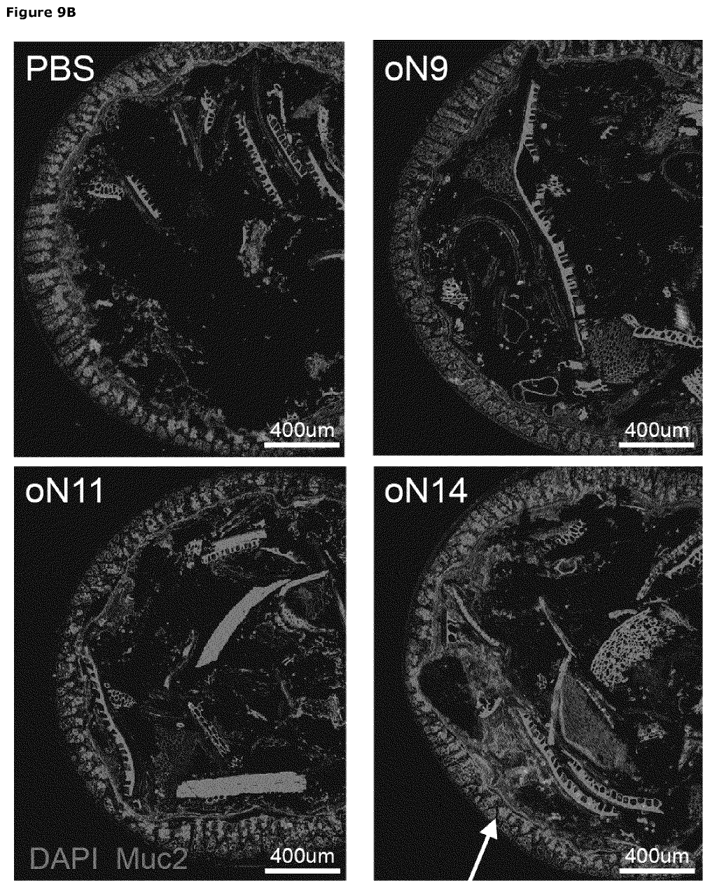

Expression of the MUC2 gene in colonic tissues derived from mice administered E. coli Nissle strain oN14 was increased ˜2-fold (p<0.05 by ANOVA) compared to control groups (FIG. 9A). Correspondingly, sections of colonic tissue immunohistochemically stained with anti-Muc2 antibodies, showed a more prominent mucosal layer in mice administered E. coli Nissle strain oN14 (FIG. 9B). Hematoxylin and Eosin staining of the colonic sections further confirmed the lack of histopathological changes associated with gut inflammation (not shown).

No difference, however, was observed in expression of goblet cell markers CDX2 or intestinal stem cell markers BM1 and LGR5 (not shown), indicating that Muc2-producing goblet cells and general intestinal turnover are unchanged. Correspondingly, no changes in gene expression levels of the inflammation markers: IL6, IL1β, NFKβ, IL17A, and TNFα by qPCR were detected in colon tissues of mice administered E. coli Nissle strain oN14 (not shown). Furthermore, no changes in colon length (p=0.66 by ANOVA) were observed, this being another indicator of chronic intestinal inflammation if found to be shortened (not shown). The expression of TJP1, TJP2, or OCLN, markers for maintenance of intestinal barrier in the colon, also remained unchanged in mice administered E. coli Nissle strain oN14 (not shown).

While no difference was observed in expression of TPH1 and SERT, an unexpected 2.8-fold increase in TPH2 expression was detected in mice administered E. coli Nissle strain oN14 compared to other groups (p<0.001 by ANOVA) in the colon (FIG. 9A). However, to change in the total number or size of EC cells (the primary producers of host 5-HT in the gut) as quantified by qPCR and by immunohistochemistry of the EC cell marker chromogranin A was detected (not shown).

Additionally, no changes were detected in the expression of IDO1, IDO2, and TDO2, in mice administered E. coli Nissle strain oN14, which encode the rate-limiting enzymes that convert L-tryptophan to N-formylkynurenine (not shown).

### Example 8

### Oral Administration of a Genetically Modified Escherichia coli Strain of the Invention Enhances Expression of Serotonin Receptors and Associated Effects in the Gut

Serotonin is a ligand for a number of 5-HT receptors, which are GPCRs mediating excitatory and inhibitory neurotransmission throughout the ENS, CNS, and the peripheral nervous system. Ligand activation of the 5HT1b receptor in the CNS has been linked to reduced aggressiveness (de Almeida et al., 2002) and, along with 5HTr1d, it is the target receptor for tryptamine-based treatment of migraines (Tepper et al., 2002). Oral administration of both E. coli Nissle strain oN11 and oN14 to mice was shown to increase HTR1B and HTR1D gene expression, suggesting a possible tryptamine-mediated effect.

The 5HT3 receptor has a wide variety of physiological roles, implicated in emesis, irritable bowel syndrome (IBS), schizophrenia, anxiety, learning, memory and addiction, and is modulated clinically by antiemetic 5HTr3 antagonists (Thompson et al., 2007). The 5HT4 receptor induces neurogenesis in the enteric nervous system, and is linked to modulation of peristalsis in the GI tract, stress-induced feeding behavior, and altered learning and memory, and depression in the CNS (Gershon et al., (2007); Lucas et al., (2007); Lamirault et al., (2001)). While 5HTr7 stimulation can improve cognition and memory (Meneses et al., 2015), and 5HTr7 antagonism can resolve antidepressive and anti-psychotic behaviors in the CNS (Roth, B. L. et al., 1994), its function in the gut is not well understood although it has been linked to IBS and inflammatory bowel disease (IBD) (Guseva, D. et al., 2014). In view of the diversity and magnitude of the detected changes in gene expression of 5-HT receptors it is plausible that E. coli strain oN14 elicits physiological responses in the host.

Upregulated expression of HTR3, HTR4 and HTR7 genes was detected in the oN14 cohort only, indicating a specific serotonin-mediated response.

Additionally oral administration of E. coli Nissle strain oN14 is shown to decrease total GI transit, consistent with serotonin being known to modulate GI motility by inducing both intraluminal pressure to cause peristaltic reflex and the colonic migrating motor complexes that sweep through the intestine during intervals of fasting.

Although changes in gut motility and bacterially-produced biogenic amines might affect the gut microbiome, no significant change in the gut microbiome were detected between mice cohorts as a result of administration of E. coli Nissle strains oN14, oN11, oN9 or PBS alone, based on a 16S metagenomic sequencing of their fecal matter (not shown). Together, administration of E. coli Nissle strains oN14 and oN11 elicits a number of physiological responses in the gut, including host 5-HT receptor gene expression and GI motility, while leaving the resident microbial community undisturbed.

8.1 Methodology

8.1.1 Oral administration: as described in 6.1.1.

8.1.2 Gene expression analysis: as described in 7.1.1.

8.1.3 Total gastrointestinal transit: Carmine red, which cannot be absorbed from the lumen of the gut, was used to study total GI transit time. A 6% solution of carmine red (300 μl; Sigma-Aldrich) suspended in 0.5% methylcellulose (Sigma-Aldrich) was administered by gavage through a 21-gauge round-tip feeding needle. The time at which gavage took place was recorded as T0. After gavage, fecal pellets were monitored at 10-minute intervals for the presence of carmine red. Total GI transit time was considered as the interval between T0 and the time of first observance of carmine red in stool.

8.1.4 Metagenomic 16S sequencing and analysis: Fecal pellets were collected from mice at Day 0 and Day 10 and immediately stored at −20□C until paired-end sequencing. Genomic DNA was extracted from fecal pellets using the Epicenter gram positive kit plus an initial bead beating step with 0.1 mm Zirconia beads. PCR amplification of the 16S rRNA V4 region and multiplexed barcoding of samples were done in accordance with previous protocols57. The V4 region of the 16S rRNA gene was amplified with 1× NEBNext q5 Hot Start HiFi PCR Master Mix using custom primers according to the method from Kozich et al., (2013). Sequencing was done with the Illumina MiSeq system (300V2 kit). Sequenced pair reads were prepared using USEARCH v10.0.240_i86osx64. Forward and reverse reads were paired and filtered to a minimum length of 240 with maximum expected error of 1. Sequences were dereplicated, clustered with minimum cluster size of 2, and mapped to OTUs at 97% identity58. OTU taxonomy was assigned with the RDP classifier59. Sequences aligned using PyNAST within Conda with QIIME1, python 2.7, matplotlib 1.4.3. Tree built with FastTree read with RStudio package ape. OTU data analysis and visualization preformed using Graphpad Prism and RStudio with gglot2 and phyloseq packages. Statistical significance performed by ANISOM using RStudio with phyloseq and vegan packages.

8.2 Results

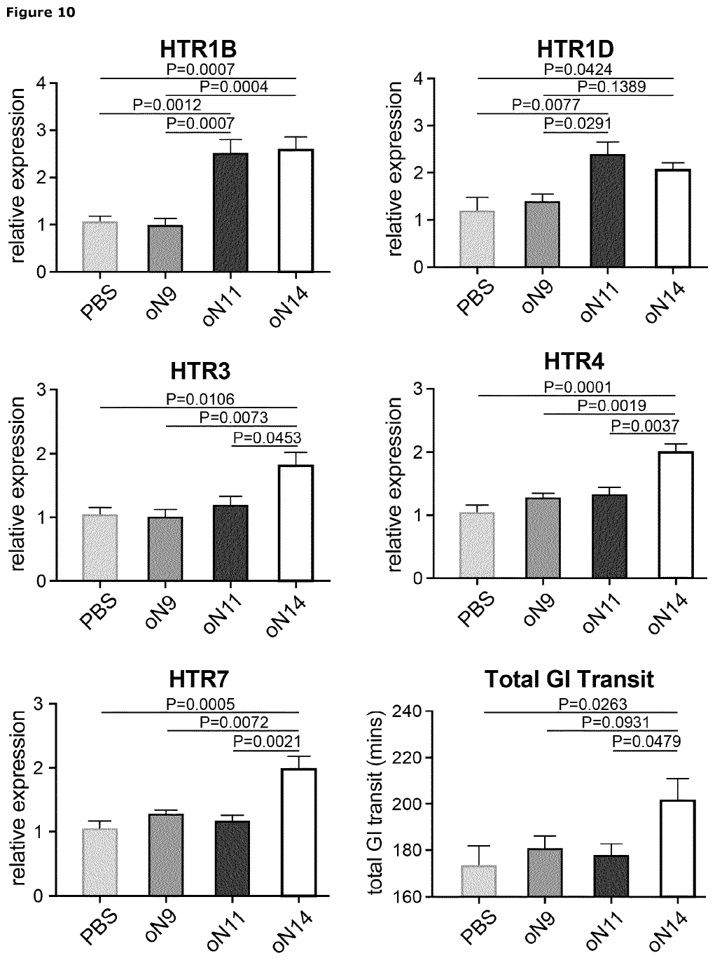

An increase in expression of HTR1B and HTR1D genes was detected in mice administered E. coli strains oN11 and oN14 (both are p<0.05 by ANOVA) (FIG. 10a, b), while increased expression of HTR3, HTR4 and HTR7 genes was only observed in colons of mice administered E. coli strain oN14 (all are p<0.01 by ANOVA) (FIG. 10c-e).

Furthermore, administration of E. coli strain oN14 specifically decreased total GI transit time by as much as 15% (p<0.05 by ANOVA) in treated mice as compared to control cohorts (FIG. 10). 16S metagenomic sequencing on fecal matter derived from mice cohorts following administration of E. coli Nissle strains oN14, oN11, oN9 or PBS alone, at Day 0 and Day 11 revealed no significant change in the gut microbiome between cohorts from Day 0 to Day 11 (R: 0.07403, significance: 0.155, by ANISOM) (not shown).

### Example 9

### Oral Administration of a Genetically Modified Escherichia coli Strain of the Invention Reduced Anxiety in Mice

Oral administration of genetically modified E. coli oN strains of the invention are shown to induce behavioral changes in treated mice, that may be mediated through the gut-brain axis or through an increase in peripheral and/or brain 5-HT levels. Two well-established methods, the Forced Swim Test (FST) and the Open Field Test (OFT) were employed to demonstrate these behavioral changes. The FST, commonly used to assess efficacy of anxiolytics and antidepressants in rodents, quantifies behavioral despair by recording the time spent immobile in a water filled container as a measure of hopelessness to escape the stressful environment. The OFT measures anxiety levels and willingness to explore in a stressful environment by detecting total fecal boli, total distance traveled, and time spent exploring the inner and outer zones of a 50×50 cm white, well-lit, open field.

Results of these tests demonstrate that oral administration of E. coli strain oN14 to mice has a therapeutic effect on anxiety-related disorders.

9.1 Methodology

9.1.1 Oral administration: as described in 6.1.1

9.1.2 Forced Swim Test: Each mouse was placed in clear cylindrical tanks (30 cm height×20 cm) with 15 cm of water at 23-25° C. for 6 minutes and recorded, as described by Ferguson (2001). Mice were then towel dried and placed under a heat lamp to dry to prevent hypothermia and then placed back in their original cage. Recordings were de-identified and a blinded observer, scored recordings for the time spent immobile during the last 4 minutes of the 6-minute test, as described by Can et al., (2012).

9.1.3 Open Field Test: Each mouse was placed in a white, open-roof, well lite, 50 cm (length)×50 cm (width)×38 cm (height) chamber and recorded for 10 minutes following previously described methods (Spohn, S. N. et al., 2016). Mice were then placed back into their original cage. Fecal pellets produced during the 10-minute test were counted. Recordings were then post processed and analyzed using BehaviorCloud as previously described (Spohn, S. N. et al., 2016). Mouse tracking software recorded total path of movement of each mouse, total distance traveled, time spent in the inner zone defined as the inner 40 cm square and outer zone defined as the outer 10 cm border closest to the walls, number of times entering the inner zone, and distance traveled within the inner zone.

9.2 Results

The FST and OFT was performed on mice cohorts following administration of E. coli Nissle strains oN14, oN11, oN9 or PBS alone.

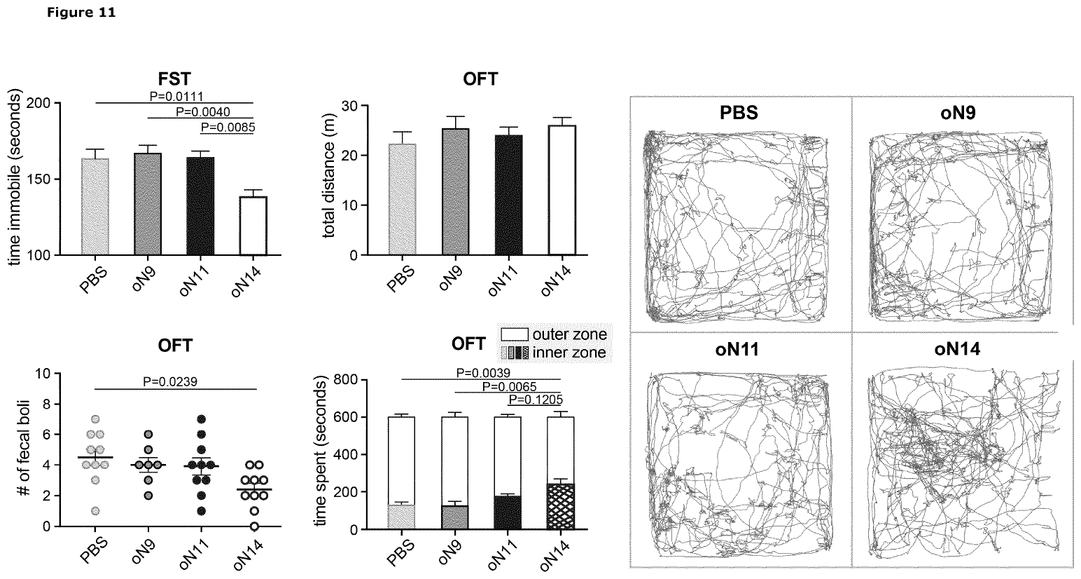

In the FST, mice cohorts administered E. coli Nissle strain oN14 exhibited a statistically reduced immobility time during FST by up to 17% (p=0.004 by ANOVA) as compared to the control cohorts administered E. coli strain oN (FIG. 11a). This result was robustly replicated in a separate and independent animal cohort (not shown).

In the OFT, mice cohorts administered E. coli Nissle strain oN14 showed a decreased number of fecal boli during the ten-minute test (FIG. 11b), which could be associated with reduced stress or the decreased total GI transit (FIG. 10f). While wild-type mice naturally prefer closed, dark, tight spaces, mice cohorts administered E. coli Nissle strain oN14 spent an increased amount of time exploring the inner 40×40 cm zone and decreased time spent near the edges along the 10×10 cm outer zone (FIG. 11d, e), with no change in total distance traveled (FIG. 11c). These observed effects in the OFT were confirmed by an OFT on another cohort of mice (not shown). Additional OFT on mice obtained from a different commercial vendor (Jackson Laboratory) having different initial gut microbiota also gave similar physiological and behavioral results as above (not shown).

### Example 10

### Efficacy of Oral Administration of a Genetically Modified Escherichia coli Strain of the Invention Matches SSRIs in Mice

Fluoxetine, sold under the brand names Prozac and Sarafem among others, is a commonly used antidepressant of the selective serotonin reuptake inhibitor (SSRI) class. It is used to treat e.g. major depressive disorder, post-traumatic stress syndrome (PTSD), and other indications. The efficacy of oral administration of genetically modified E. coli oN strains of the invention is shown to correspond to administration of fluoxetine, by the ability to induce behavioral changes in treated mice indicative of a therapeutic reduction in anxiety or stress.

10.1 Methodology:

10.1.1 Administration: as described for oral administration in 6.1.1, but with the following modifications: The mice were administered sterile PBS only (PBS) or freshly-grown 10⁹ cells of E. coli Nissle strain designated oN14 (E. coli Nissle strain oN comprising pMUT14-ser) in sterile PBS (oN14) by oral gavage. Additionally, the mice were administered a 200 μl intraperitoneal (I.P.) injection of saline or the SSRI, fluoxetine at 10 mg/kg body weight in saline. The mice received the oral and IP administered co-treatments every 24 hrs at the same time of day for 21 days. Accordingly, the four mice treatment groups received: IP: saline/Gavage: PBS (negative control); IP: Fluoxetine/Gavage: PBS (SSRI positive comparator), IP: saline/Gavage: oN14 (oN14 positive comparator); and IP: Fluoxetine/Gavage: oN14 (positive combination comparator). 24 hours after the last administration, the mice were subjected to motility and behavioral testing.

10.2 Results

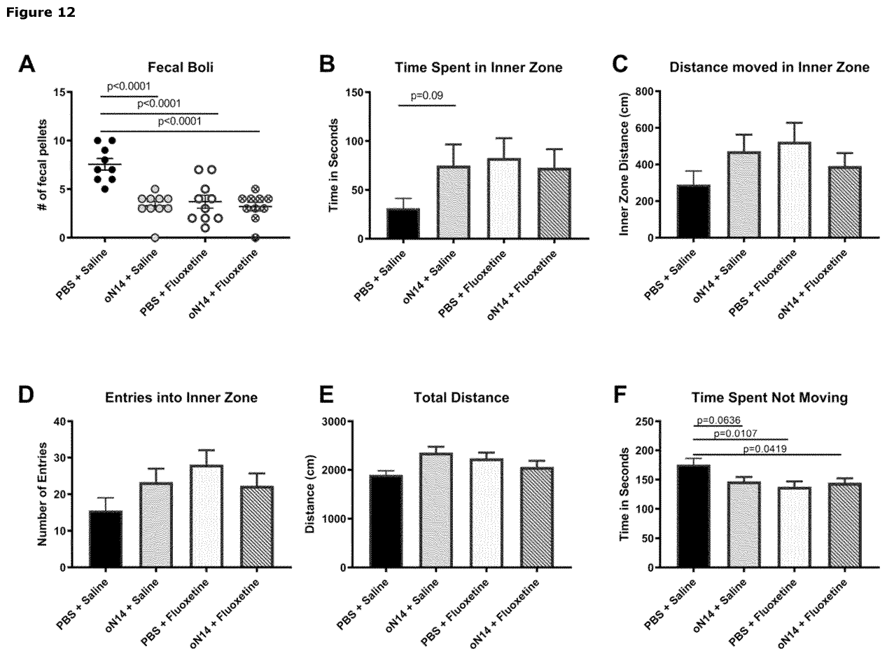

The phenotypes exhibited by mice after 21 days treatment with oN14 (IP: saline/Gavage: oN14) closely resemble those treated with Fluoxetine (IP: Fluoxetine/Gavage). As seen in FIG. 12, A) A reduction in fecal boli produced during the OFT is observed both with oN14, Fluoxetine or the combination therapy (IP: Fluoxetine/Gavage: oN14), indicating a reduction in stress level leading to less defecation. Further, in FIG. 12 B)-E) the time spent and distance travelled in the inner zone, as well as the total number of entries in the inner zone, are all increased by either oN14 or fluoxetine treatment. An increase of these parameters is interpreted as a reduction in anxiety or stress, and demonstrates an increased willingness to explore in an unknown environment. In the Forced Swim Test (FIG. 12F), the time spent not moving is measured as an indication of hopelessness/depression. oN14 and Fluoxetine treatment both significantly reduce the time of immobility, indicating similar efficacy. oN14 treated mice showed similar effects on time spent in the inner zone, entries in the inner zone, and distance in the inner zone as compared to mice treated with Fluoxetine (FIG. 12). The combination of treatment with oN14 and treatment with fluoxetine yielded a small reduction in the total effect when compared to the individual groups treated with only oN14 or fluoxetine.

### Example 11

### Oral Administration of a Genetically Modified Escherichia coli Strain of the Invention Increases Plasma and Urine Serotonin

Oral administration of the 5-HTP-producing E. coli Nissle strain oN10 to mice was shown to increase plasma serotonin and urine serotonin and 5-HTP concentrations in mice.

11.1 Methodology

11.1.1 Oral administration: as described in 6.1.1. with the following modifications: Animals received Streptomycin (5 g/L) in the drinking water from 3 days before gavage and throughout the experiment. A single oral gavage of 108 cells of either oN10 or a control EcN strain without tryptophan hydroxylase (EcN_Control). Animals were treated with the TDC inhibitor Carbidopa via intraperitoneal injection (IP) every 24 h, and a fresh fecal sample was collected daily for 7 days, after which the animals were euthanized. Plasma samples were taken on day 2 and day 7 after gavage.

11.1.2 In vivo metabolite analysis: Plasma, tissue, gut content, urine and fecal samples were analyzed for serotonin, 5-hydroxytrytophan, tryptamine, 5-Hydroxyindoleacetic acid (5-HIAA) and tryptophan. For plasma: blood samples were collected via the submandibular vein on day 2 (and by heart puncture day 8) post gavage, kept on ice for 10 min in Li-Heparin Microtainers, and then plasma was separated by centrifugation at 10000 g at 4° C. for 90 sec. and plasma snap-frozen at −80 C. An internal standard buffer containing 0.9% NaCl, 0.2% Ascorbic acid, and 20 mg/L C-13-labelled tryptophan was added after thawing the samples, and proteins were precipitated using methanol extraction. After drying samples using a vacuum centrifuge, they were reconstituted in 50 ul ddH₂O for LC-MS/MS analysis. For tissue analysis: colon tissue was weighed and immediately frozen on liquid nitrogen after collection and cryofractured using stainless steel microvials (Biospec 2007) with a single 6.34 mm stainless steel bead (Biospec 11079635ss) beat for 30 s in BeadBeater (Biospec 112011). Urine was collected on day 2 and day 6 post gavage, and immediately snap-frozen at −80° C. An internal standard buffer containing 0.9% NaCl, 0.2% Ascorbic acid, and 20 mg/L C-13-labelled tryptophan was added after thawing the samples, and proteins were precipitated using methanol extraction. After drying samples using a vacuum centrifuge, they were reconstituted in 50 ul ddH₂O for LC-MS/MS analysis.

11.2 Results

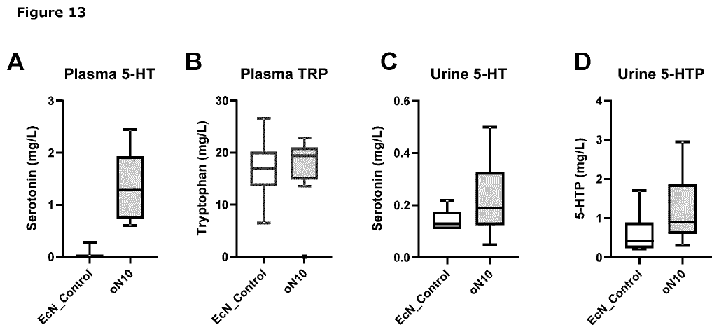

A significant increase in 5-HT concentration was observed in the plasma with oN10 compared to a control strain without TPH expression (p<0.001 by two-tailed t-test), demonstrating successful oN10-derived production of 5-HTP in vivo (FIG. 13A). The amount of 5-HTP produced by oN10 was sufficient to elevate peripheral plasma serotonin concentrations while leaving tryptophan concentrations unaffected (FIG. 13B), indicating a specific effect of oN10 on serotonin metabolism within physiologically relevant levels. Plasma concentrations of 5-HTP were below quantification levels in all samples; however, since 5-HTP is known to cross the blood-brain-barrier, peripherally produced 5-HTP is able to increase serotonin biosynthesis in the brain, with potential therapeutic effects on mood, sleep, anxiety and other disorders (Turner et al., 2006). The oN10 strain presents a mechanism to deliver 5-HTP from within the GI tract at a constant dosage, circumventing undesirable effects of dosage-dependent fluctuations in neurotransmitters like 5-HTP and 5-HT.

Urine 5-HT and 5-HTP concentrations were also increased with oN10 compared to the control group (FIGS. 13C and D, respectively), showing increased serotonergic metabolism with oN10.

### Example 12

### Genetically Modified Saccharomyces cerevisiae Cells Express a Biosynthetic Pathway for 5-HT Production

Saccharomyces cerevisiae (S. cerevisiae) was genetically modified by the introduction of recombinant genes to establish a synthetic serotonin pathway comprising enzymes to catalyze the two consecutive metabolic steps of converting TRP to 5-HTP and then 5-HTP to 5-HT (FIG. 1). Identification of the optimal combination of enzymes to catalyze these two metabolic steps was determined by expressing and determining the 5-HT yield obtainable from the combination of TPH and TDC genes.

12.1 Methodology

Modified S. cerevisiae cells were engineered as follows, with reference to Table 1:

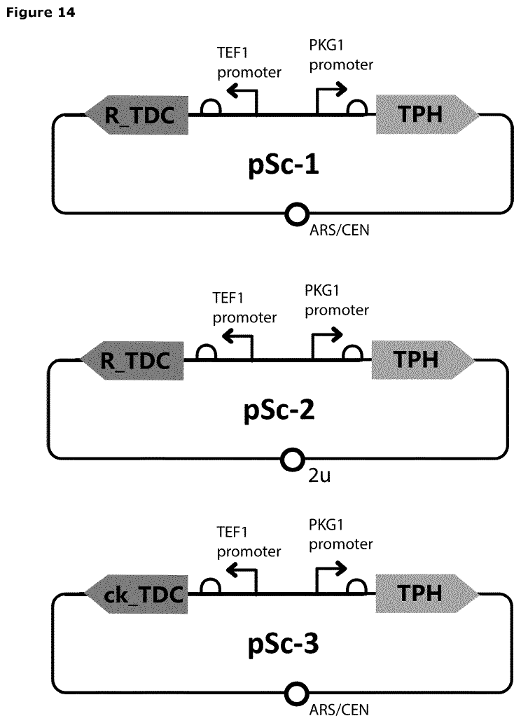

The host strain, S. cerevisiae was obtained from Mans et al., (2015). Genes encoding TPH proteins derived from mammalian, plant or bacterial sources: mouse TPH1 (M1) and rice TDC (R) were the same as in 1.1.1. A different TDC gene from the bacterium Candidatus Koribacter versatilis Ellin345 (ck) was also tested. The pathway genes were cloned into either the yeast 2μ plasmid backbone, or the ARS/CEN backbone (see FIG. 14). The M1 gene was cloned under control of the PKG1 promoter, and the TDC genes (either rice TDC or ck TDC) were controlled by the TEF1 promoter. The plasmids were subsequently transformed into the host strain, by employing standard cloning and transformation procedures known in the art.

Growth and in vitro metabolite production was determined as follows: Yeast strains were grown in Synthetic Complete Dropout (SC) medium (Yeast Nitrogen Base Without Amino Acids 6.7 g/L, glucose 20 g/L, drop-out mix 2 g/L). The drop-out mix used lacked histidine, thereby maintaining the plasmids. The negative control (containing no plasmids) was grown with 20mg/L histidine. Three single colonies were picked from each strain, grown in 2 ml μl medium in 24 deep-well plates and shaken at 250 rpm at 30° C. for 48 hours. The main culture was inoculated to OD₆₀₀=0.05 in 25 ml SC medium+500 mg/L tryptophan in shake flasks, and cells were grown for 72 h under the same conditions. Afterwards, the culture supernatant was separated from cells by centrifugation at 17000×g for 2 minutes, and frozen at −20° C. until analysis by HPLC. All data shown are mean+/−SD from at least 3 biological replicates.

Metabolite production was quantified by LC-MS as in 1.1.3

12.2 Results:

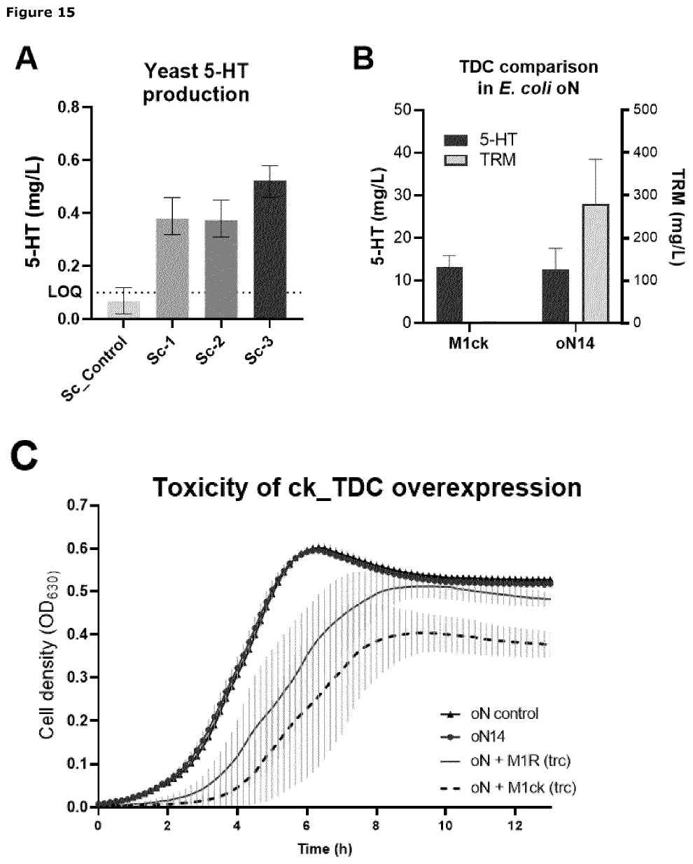

Genetically modified strains of the host, S. cerevisiae, engineered to express the mouse TPH1 (M1), in combination with TDC from rice (R) [or a bacterial TDC gene from Candidatus Koribacter versatilis Ellin345 (ck_TDC) produced significant amounts of serotonin (FIG. 15A). While in the control strain without TPH or TDC gene expression (Sc_Control), no serotonin production is detected (p<0.0018, ANOVA), strains expressing mouse TPH1 and R-TDC (pSc-1 and pSc-2) or ck-TDC (pSc-3) (see table 1) produced circa 0.38+/−0.07 mg/L and 0.52+/−0.06 mg/L serotonin, respectively. The ck-TDC gene can also functionally replace the R-TDC gene and enables production of serotonin in E. coli constructs (FIG. 15B), but strong overexpression of ck-TDC causes cellular toxicity in oN (FIG. 15C) leading to a reduction in growth rate and final biomass yield compared to R-TDC expression.

### Example 13

### Genetically Modified Bacillus subtilis 168 Cells Express a Biosynthetic Pathway for 5-HT Production

Bacillus subtilis (B. subtilis) was genetically modified by the introduction of recombinant genes to establish a synthetic serotonin pathway comprising enzymes to catalyze the two consecutive metabolic steps of converting TRP to 5-HTP and then 5-HTP to 5-HT (FIG. 1). Identification of the optimal combination enzymes to catalyze these two metabolic steps is determined by expressing and determining the 5-HT yield obtainable from the combination of TPH and TDC genes.

13.1 Methodology

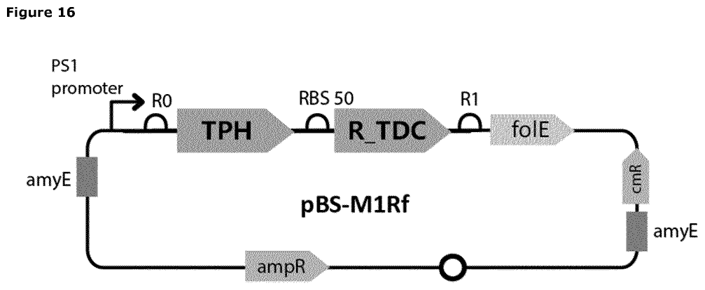

The host strain, B. subtilis 168, obtained from the German Collection of Microorganisms and Cell Cultures GmbH (DSMZ) [NC_000964], was genetically engineered as follows, with reference to Table 1: Genes encoding TPH proteins derived from mammalian or plant sources: mouse TPH1 (M1) and rice TDC (R) were the same as in 1.1.1. The tested TPH and TDC gene combination, together with the T198I variant of the E. coli folE gene, were cloned into an integrative plasmid (derived from pDG364, see Table 1) to facilitate genomic integration of the pathway operon. The genes were placed in a synthetic operon consisting of a strong, Bacillus-specific promoter (Ps11), a strong RBS (R01) for M1, a medium-strength RBS for TDC (RBS 50 from Table 1), and a strong RBS (R11) for folE(T198I). Plasmid pBs-M1Rf contained the mouse TPH1, rice TDC, and folE(T198I) genes (FIG. 16).

Cloning was performed in E. coli Top10 by employing standard molecular biology methods. The plasmids were subsequently transformed into the host strain, by employing standard cloning and transformation procedures known in the art. Correct pathway integration into the B. subtilis 168 genome at the amyE gene locus was verified by Sanger sequencing.

Growth and in vitro metabolite production was determined as follows: B. subtilis 168 strains were grown in Lysogeny Broth (LB) medium containing 0.2% (w/v) glucose and 50 mg/L of L-tryptophan. Three single colonies were picked from each strain, grown in 300 μl medium in 96 deep-well plates and shaken at 250 rpm at 37° C. for 16 hours. The main culture was inoculated by diluting this pre-culture 1:100 into fresh medium, and cells were grown for 24 h under the same conditions. Afterwards, the culture supernatant was separated from cells using a 0.2 μm pore size filter, and frozen at −20° C. until analysis by LC-MS. All data shown are mean+/−SD from at least 3 biological replicates.

Metabolite production was quantified by LC-MS as in 1.1.3.

13.2 Results:

Genetically modified strains of the host, B. subtilis 168, engineered to express the mouse TPH1 (M1), in combination with TDC from rice (R) and E. coli folE(T198I) are expected to produce of serotonin and 5-HTP.

No 5-HTP or 5-HT is produced using the wild-type Bacillus subtilis 168 strain, while expression of tryptophan hydroxylase M1, together with folE(T198I), will enable 5-HTP production. Expression of M1_TPH, folE(T198I) and R_TDC is expected to result in production of serotonin.

TABLE 1
Strains, plasmids and genes used in the examples

| Strains* | | |
|---|---|---|
| Name | Genetic features | Source |
| N | Wild-type E. coli Nissle 1917 (EcN) | Ardeypharm |
| | | GmbH |
| NΔ2 | EcN ΔtrpR, ΔtnaA, attTn7::GFP | this study |
| NΔ3 | EcN ΔtrpR, ΔtnaA, ΔinfA, attTn7::GFP | this study |
| NΔ2-pMUT1 | EcN ΔtrpR, ΔtnaA, attTn7::GFP, cured of pMUT1 | this study |
| ON | EcN MrpR, MnaA, folE(T1981), attTn7::GFP , cured | this study |
| | of pMUT1 | |
| ON9 | ON + pMUT09-ctrl | this study |
| ON10 | ON + pMUTM10-5htp | this study |
| ON11 | ON + pMUT11-trm | this study |
| ON14 | ON + pMUT14-ser | this study |
| Sc | Saccharomyces cerevisiae strain derived from | Mans, R. et al. |
| | CEN.PK2-1C. Genotype: MATa; his3Dl; leu2-3_112; | 2015 |
| | ura3-52; trp1-289 | |
| Bs | Wild-type Bacillus subtilis 168 | DSMZ |
| EcN_Control | EcN_Tn7::GFP, folE(T198I), AtyrR, StrepR + pMh- | this study |
| | empty | |

*Sequences for the ΔtrpR (SEQ ID NO.: 27), ΔtnaA (SEQ ID NO.: 30), ΔinfA (SEQ ID
NO.: 33), folE(T1981) (SEQ ID NO.: 3), and attTn7::GFP (SEQ ID NO.:34) can be
found in the sequence list.

| Plasmids ** | | |
|---|---|---|
| Name | Genetic features | Source |
| pMUT1 | Native plasmid of E. coli Nissle 1917 | Genbank |
| | | A84793.1 (SEQ |
| | | ID NO.: 35) |
| pMUT | nucleotides 1-1323 and 1664-3117 from pMUT1 | this study |
| pUC-H2R | P_{BBa}_323107, human CTPH2 (RBS = 5000), rice TDC | GeneArt®/ |
| | (RBS = 10000), infA, pUC backbone | this study |
| pUC-H2Rₒₚₜ | P_{BBa}_323107, human CTPH2 (RBS = 5000), rice TDC | this study |
| | (RBS = 700), infA, pUC backbone | |
| pUC-H2R_{D} | P_{BBa}_323107, human CTPH2 (RBS = 5000), rice TDC | this study |
| | (RBS = 50), infA, pUC backbone | |
| pUC-HIR | P_{BBa323107}, human cTPHI (RBS = 5000), rice TDC | this study |
| | (RBS = 10000), infA, pUC backbone | |
| pUC-MIR | P_{BBa}_323107, mouse cTPHI (RBS = 3700), rice TDC | this study |
| | (RBS = 10000), infA, pUC backbone | |
| pUC-M2R | P_{BBa}_323107, mouse cTPH2 (RBS = 5000), rice TDC | this study |
| | (RBS = 10000), infA, pUC backbone | |
| PUC-H2G | P_{BBa}_323107, human cTPH2 (RBS = 5000), guinea pig | this study |
| | TDC (RBS = 10000), infA, pUC backbone | |
| pUC-H2C | P_{BBa}_323107, human cTPH2 (RBS = 5000), Catharanthus | this study |
| | roseus TDC (RBS = 10000), infA, pUC backbone | |
| pMUT-H2R | PBBa_323107, human CTPH2 (RBS = 5000), rice TDC | this study |
| | (RBS = 700), pMUT1 backbone | |
| pMUT-MIR | P_{BBa}_323107, mouse cTPHI (RBS = 2300), rice TDC | this study |
| | (RBS = 250), pMUT1 backbone | |
| pMUT09-ctrl | P_{BBa}_323101, pMUT1 backbone, hok/sok locus | this study |
| pMUT10-5htp | P_{BBa}_323107, human cTPH2 (RBS = 10000), pMUT1 | this study |
| | backbone, hok/sok locus | |
| pMUT11-trm | P_{BBa}_323101, rice TDC (RBS = 700), pMUT1 backbone, | this study |
| | hok/sok locus | |
| pMUT14-ser | P_{BBa}_323101, mouse THP1 (RBS = 18000), rice TDC | this study |
| | (RBS = 700), pMUT1 backbone, hok/sok locus | |
| pDG364 | B. subtilis integrative plasmid for | Hoa, N. T. et al |
| | insertion into the genomic amyE locus | (2002) |
| pBs-MIRf | P_{PS1}, mouse TPHI (RBS = R0*), rice TDC (RBS = | this study |
| | 50), folE(T198I) (RBS = R1*), backbone from | * Guiziou, S. et |
| | pDG364 | al. (2016) |
| pARS/CEN | pRS413U | NEB |
| p2u | pESC-HIS3-ccdB-USER | Agilent Catalog |
| | | #217451 |
| pSc_1 | P_{TEF1}, rice TDC, P_{PKG1}, mouse TPHI, ARS/CEN | this study |
| | backbone | |
| pSc_2 | P_{TEF1}, rice TDC, P_{PKG1}, mouse TPHI, 2u backbone | this study |
| pSc_3 | P_{TEF1}, ckTDC, P_{PKG1}, mouse TPHI, ARS/CEN | this study |
| | backbone | |

** Sequences for promoters P_{BBa}_J23101 and P_{BBa}_J23107 are found in the sequence list:
SEQ ID NO.: 36 and 37.

| Genes/Proteins | | |
|---|---|---|
| Wt folE (GCHI) | GTP cyclohydrolase 1 (SEQ ID NO.: 2) | Locus tag |
| | | ECOLIN_12170, |
| | | Protein |
| | | AID79293.1 |
| infA | translation initiation factor IF-1 (SEQ ID NO.: 32), | Locus tag |
| | encoded by native gene on E.coli Nissle genome | ECOLIN_04590, |
| | (SEQ ID NO.: 31) | Protein |
| | | AID77906.1 |
| hok/sok genes | toxin-antitoxin plasmid stability element | GenBank: |
| | | MK134376, bp |
| | | 58002-58601 |
| H1 = hTPHI | TPHI from Homo sapiens, catalytic domain: amino | UniProt ID |
| | acids 103 to 416 | P17752 (SEQ |
| | | ID NO.: 9, 10) |
| H2 = hTPH2 | TPH2 from Homo sapiens, catalytic domain: amino | UniProt ID |
| | acids 144 to 462 | Q8IWU9 (SEQ |
| | | ID NO.: 11, 12) |
| M1 = mTPHI | TPHI from Mus musculus, catalytic domain: amino | UniProt ID |
| | acids 106 to 419 | P17532 (SEQ |
| | | ID NO.: 5, 6) |
| M2 = mTPH2 | TPH2 from Mus musculus, catalytic domain: amino | UniProt ID |
| | acids 142 to 460 | Q8CGV2 (SEQ |
| | | ID NO.: 7, 8) |
| R = rTDC | TDC from Oryza sativa subsp. japonica (Rice) | Uniprot ID |
| | | Q6ZJK7(SEQ |
| | | ID NO.: 17, 18) |
| G = gTDC | TDC (called AADC, aromatic-amino-acid | UniProt ID |
| | decarboxylase in mammals) from Cavia porcellus | P22781 (SEQ |
| | (Guinea pig) | ID NO.: 15, 16) |
| C = CTDC | TDC from Catharanthus roseus (Madagascar | UniProt ID |
| | periwinkle) | P17770 (SEQ |
| | | ID NO.: 13, 14) |
| ck = ckTDC | TDC from Candidatus Koribacter versatilis Ellin345 | Sequence ID: |
| | | ABF41161.1 |
| | | (SEQ ID No.: |
| | | 110, 111) |

Bacillus subtilis promoter and RBS sequences

| | | |
|---|---|---|
| P_PS1 | actgcgtcaatacacgttgacactcttttgAAAGTGtgttaaattatcag | SEQ ID No.: |
| (promoter) | | 105 |
| R0(RBS) | GATTAACTAATAAGGAGGACAAAA | SEQ ID No.: |
| | | 106 |
| R1(RBS) | GCTCTTAAGGAGGATTTTAGA | SEQ ID No.: |
| | | 107 |

S. cerevisiae promoter sequences

| | |
|---|---|
| P_TEF1 | SEQ ID No.: |
| | 108 |
| P_PKG1 | SEQ ID No.: |
| | 109 |

TABLE 2
RBS variants used in the examples

| | | | |
|---|---|---|---|
| RBS ID in | | Translation | SEQ |
| text/ | | Initiation | ID |
| figures | RBS Sequence 5′-3′ | Rate (au) | NO. |

mouse TPH1 RBS variants

| | | | |
|---|---|---|---|
| M1 RBS = 63000 | AGGTATTATGCTAGCAGCTCATCAATACCCC | 63141 | 38 |
| | CCCAATAAGGAGGATTAAG | | |
| M1 RBS = 35000 | AGGTATTATGCTAGCAGCTCATCAATACCCC | 35174 | 39 |
| | CCCAACAAGGAGGATTAAG | | |
| M1 RBS = 18000 | AGCTCATCAATACCCCCCCAATAAGGAGGAT | 18000 | 40 |
| | TAAG | | |
| M1 RBS = 13000 | AGGTATTATGCTAGCAGCTCATCAATACCCC | 13670 | 41 |
| | CCCAATCAGGAGGATTAAG | | |
| M1 RBS = 4200 | AGGTATTATGCTAGCAGCTCATCAATACCCC | 4242 | 42 |
| | CCCAACCAGGAGGATTAAG | | |
| M1 RBS = 2300 | AGCTCATCAATACCCCCCCAAAAATAAGGAT | 2300 | 43 |
| | TAAG | | |

human TPH2 RBS variants

| | | | |
|---|---|---|---|
| H2 RBS = 36000 | AGCTCATCAATACCCCCCCATAAGCGAGGTT | 36793 | 44 |
| | TAAG | | |
| H2 RBS = 10000 | AGCTCATCAATACCCCCCCATAACCGAGGTT | 10818 | 45 |
| | TAAG | | |
| H2 RBS = 5200 | AGCTCATCAATACCCCCCCATAAGCAAGGTT | 5265 | 46 |
| | TAAG | | |
| H2 RBS = 5000 | AGCTCATCAATACCCCCCCAAAAATAAGGAT | 5000 | 47 |
| | TAAG | | |
| H2 RBS = 3000 | AGCTCATCAATACCCCCCCATAACCAAGGTT | 2933 | 48 |
| | TAAG | | |

rice TDC RBS variants

| | | | |
|---|---|---|---|
| R RBS = 10000 | GAAGAAATTATCAGAGAGAGGGAAGGTAAC | 10000 | 49 |
| | AC | | |
| R RBS = 3800 | TACCCGTAGCTAAGAAGAAATTATCAGAGAG | 3877 | 50 |
| | AGGGTAGGTAACAC | | |
| R RBS = 1800 | TACCCGTAGCTAAGAAGAAATTATCAGAGAG | 1887 | 51 |
| | ACGGTAGGTAACAC | | |
| R RBS = 700 | TACCCGTAGCTAAGAAGAAATTATCAGAGAG | 733 | 52 |
| | AGGGTCGGTAACAC | | |
| R RBS = 250 | AAGCTAAGAAGAAATTATCAGAGGGTCGGT | 250 | 53 |
| | AACAC | | |
| R RBS = 50 | TACCCGTAGCTAAGAAGAAATTATCAGAGAG | 49 | 54 |
| | ACGGTCGGTAACAC | | |

TABLE 3
qPCR primers used in the examples

| | | SEQ | | SEQ |
|---|---|---|---|---|
| | Forward Primer: | ID | Reverse Primer: | ID |
| Gene | Sequence | NO. | Sequence | NO. |
| CYCLO | 5′-GACGAAG | 55 | 5′-AATCAGG | 56 |
| | GTAGCCAGTC | | CCTGTGGAAT | |
| | ACAAG-3′ | | GTGAG-3′ | |
| MUC2 | 5′-ATGCCCA | 57 | 5′-GTAGTTT | 58 |
| | CCTCCTCAAA | | CCGTTGGAAC | |
| | GAC-3′ | | AGTGAA-3′ | |
| CDX2 | 5′-CAAGGAC | 59 | 5′-GTAACCA | 60 |
| | GTGAGCATGT | | CCGTAGTCCG | |
| | ATCC-3′ | | GGTA-3′ | |
| BM1 | 5′-AAATCCC | 61 | 5′-CTTGCTG | 62 |
| | CACTTAATGT | | GTCTCCAAGT | |
| | GTGTCC-3′ | | AACG-3′ | |
| LGR5 | 5′-GGACCAG | 63 | 5′-CAGAGGC | 64 |
| | ATGCGATACC | | GATGTAGGAG | |
| | GC-3′ | | ACTG-3′ | |
| IL6 | 5′-CTGCAAG | 65 | 5′-AGTGGTA | 66 |
| | AGACTTCCAT | | TAGACAGGTC | |
| | CCAG-3′ | | TGTTGG-3′ | |
| IL1β | 5′-GCAACTG | 67 | 5′-ATCTTTT | 68 |
| | TTCCTGAACT | | GGGGTCCGTC | |
| | CAACT-3′ | | AACT-3′ | |
| NFKβ | 5′-ATGGCAG | 69 | 5′-TGTTGAC | 70 |
| | ACGATGATCC | | AGTGGTATTT | |
| | CTAC-3′ | | CTGGTG-3′ | |
| IL17A | 5′-TTTAACT | 71 | 5′-CTTTCCC | 72 |
| | CCCTTGGCGC | | TCCGCATTGA | |
| | AAAA-3′ | | CAC-3′ | |
| TNFα | 5′-CAGGCGG | 73 | 5′-CGATCAC | 74 |
| | TGCCTATGTC | | CCCGAAGTTC | |
| | TC-3′ | | AGTAG-3′ | |
| TPH1 | 5′-AACAAAG | 75 | 5′-TGTAACA | 76 |
| | ACCATTCCTC | | GGCTCACATG | |
| | CGAAAG-3′ | | ATTCTC-3′ | |
| TPH2 | 5′-GTGACCC | 77 | 5′-GGTGCCG | 78 |
| | TGAATCCGCC | | TACATGAGGA | |
| | TG-3′ | | CT-3′ | |
| SERT | 5′-CTCCGCA | 79 | 5′-CACGGCA | 80 |
| | GTTCCCAGTA | | TAGCCAATGA | |
| | CAAG-3′ | | CAGA-3′ | |
| CHGA | 5′-CAGGCTA | 81 | 5′-GCCTCTG | 82 |
| | CAAAGCGATC | | TCTTTCCATC | |
| | CAG-3′ | | TCC-3′ | |
| TJP1 | 5′-GCCGCTA | 83 | 5′-TCCCCAC | 84 |
| | AGAGCACAGC | | TCTGAAAATG | |
| | AA-3′ | | AGGA-3′ | |
| TJP2 | 5′-GTTTGCC | 85 | 5′-CTTCAAA | 86 |
| | GTTCAGCAGC | | ACCTCGGTCG | |
| | TTAG-3′ | | TCAT-3′ | |
| OCLN | 5′-TGAAAGT | 87 | 5′-CCGGATA | 88 |
| | CCACCTCCTT | | AAAAGAGTAC | |
| | ACAGA-3′ | | GCTGG-3′ | |
| IDO1 | 5′-CAAAGCA | 89 | 5′-ACAAAGT | 90 |
| | ATCCCCACTG | | CACGCATCCT | |
| | TATCC-3′ | | CTTAAA-3′ | |
| ID02 | 5′-CCTCATC | 91 | 5′-GGAGCAA | 92 |
| | CCTCCTTCCT | | TTGCCTGGTA | |
| | TTC-3′ | | TGT-3′ | |
| TD02 | 5′-AGGAACA | 93 | 5′-CTGTAGA | 94 |
| | TGCTCAAGGT | | CTCTGGAAGC | |
| | GATAGC-3′ | | CTGAT-3′ | |
| HTR1b | 5′-CGCCGAC | 95 | 5′-TAGCTTC | 96 |
| | GGCTACATTT | | CGGGTCCGAT | |
| | AC-3′ | | ACA-3′ | |
| HTR1d | 5′-ATCACCG | 97 | 5′-GCGAGAA | 98 |
| | ATGCCCTGGA | | GAGTGGAGGG | |
| | GTA-3′ | | ATG-3′ | |
| HTR3 | 5′-CCTGGCT | 99 | 5′-TGCAGAA | 100 |
| | AACTACAAGA | | ACTCATCAGT | |
| | AGGGG-3′ | | CCAGTA-3′ | |
| HTR4 | 5′-AGTTCCA | 101 | 5′-CAGCAGG | 102 |
| | ACGAGGGTTT | | TTGCCCAAGA | |
| | CAGG-3′ | | TG-3′ | |
| HTR7 | 5′-TGCGGGG | 103 | 5′-GACAAAG | 104 |
| | AGCAGATCAA | | CACACCGAGA | |
| | CTA-3′ | | TCAC-3′ | |

### REFERENCES

Baba, T. et al. Construction of Escherichia coli K-12 in-frame, single-gene knockout mutants: the Keio collection. Mol Syst Biol 2, 2006 0008 (2006).

Blin, K., Pedersen, L. E., Weber, T. & Lee, S. Y. CRISPy-web: An online resource to design sgRNAs for CRISPR applications. Synth Syst Biotechnol 1, 118-121 (2016).

Blum-Oehler, G. et al. Development of strain-specific PCR reactions for the detection of the probiotic Escherichia coli strain Nissle 1917 in fecal samples. Res Microbiol 154, 59-66 (2003).

Browning, K N. Role of central vagal 5-HT3 receptors in gastrointestinal physiology and pathophysiology. Front Neurosci.; 9: 413 (2015).

Breit, S et al., Vagus Nerve as Modulator of the Brain-Gut Axis in Psychiatric and Inflammatory Disorders Front Psychiatry; 9: 44. (2018)

Can, A., Dao, D. T., Arad, M., Terrillion, C. E., Piantadosi, S. C., Gould, T. D. The Mouse Forced Swim Test. J. Vis. Exp. (59) (2012).

Casteleyn, C., Rekecki, A., Van der Aa, A., Simoens, P. & Van den Broeck, W. Surface area assessment of the murine intestinal tract as a prerequisite for oral dose translation from mouse to man. Lab. Anim. 44, 176-83 (2010).

de Almeida, R. M. & Miczek, K. A. Aggression escalated by social instigation or by discontinuation of reinforcement (“frustration”) in mice: inhibition by anpirtoline: a 5-HT1B receptor agonist. Neuropsychopharmacology 27, 171-181 (2002).

Ferguson, J. M. SSRI Antidepressant Medications: Adverse Effects and Tolerability. Prim Care Companion J Clin Psychiatry 3, 22-27 (2001).

Gershon, M. D. & Liu, M. T. Serotonin and neuroprotection in functional bowel disorders. Neurogastroenterol Motil 19 Suppl 2, 19-24 (2007).

Gibson, D. G. et al. Enzymatic assembly of DNA molecules up to several hundred kilobases. Nat Methods 6, 343-345 (2009).

Guiziou, S. et al. A part toolbox to tune genetic expression in Bacillus subtilis. Nucleic Acids Res. 44, 7495-7508 (2016).

Guseva, D. et al. Serotonin 5-HT7 receptor is critically involved in acute and chronic inflammation of the gastrointestinal tract. Inflamm Bowel Dis 20, 1516-1529 (2014).

Hoa, N. T., Brannigan, J. A. & Cutting, S. M. The Bacillus subtilis signaling protein SpoIVB defines a new family of serine peptidases. J. Bacteriol. 184, 191-199 (2002).

Ianiro, G., et al., Role of Yeasts in Healthy and Impaired Gut Microbiota: The Gut Mycome. Curr Pharm Des.; 20(28):4565-9. (2014).

Jensen, S. I., Lennen, R. M., Herrgard, M. J. & Nielsen, A. T. Seven gene deletions in seven days: Fast generation of Escherichia coli strains tolerant to acetate and osmotic stress. Sci Rep 5, 601 17874 (2015).

Kozich, J. J., Westcott, S. L., Baxter, N. T., Highlander, S. K. & Schloss, P. D. Development of a dual index sequencing strategy and curation pipeline for analyzing amplicon sequence data on the MiSeq Illumina sequencing platform. Appl Environ Microbiol 79, 5112-5120 (2013).

Lamirault, L. & Simon, H. Enhancement of place and object recognition memory in young adult and old rats by RS 67333, a partial agonist of 5-HT4 receptors. Neuropharmacology 41, 844-853 (2001).

Li, J et al., An integrated catalog of reference genes in the human gut microbiome Nature Biotechnology volume 32, pages 834-841 (2014)

Liu, Q. et al. Discovery and Characterization of Novel Tryptophan Hydroxylase Inhibitors That Selectively Inhibit Serotonin Synthesis in the Gastrointestinal Tract. J. Pharmacol. Exp. Ther. 325, 47-55 (2008).

Lucas, G. et al. Serotonin(4) (5-HT(4)) receptor agonists are putative antidepressants with a rapid onset of action. Neuron 55, 712-725 (2007).

Mans, R et al., (2015) CRISPR/Cas9: A molecular Swiss army knife for simultaneous introduction of multiple genetic modifications in Saccharomyces cerevisiae. FEMS Yeast Research, Volume 15, Issue 2; doi.org/10.1093/femsyr/fov004.

McKenzie, G. J. & Craig, N. L. Fast, easy and efficient: site-specific insertion of transgenes into enterobacterial chromosomes using Tn7 without need for selection of the insertion event. BMC Microbiol 6, 39 (2006).

Mehrer, C. R., Incha, M. R., Politz, M. C. & Pfleger, B. F. Anaerobic production of medium-chain fatty alcohols via a beta-reduction pathway. Metab Eng 48, 63-71 (2018).

Meneses, A. et al. 5-HT7 receptor activation: procognitive and antiamnesic effects. Psychopharmacology (Berl) 232, 595-603 (2015).

Meneses, A. Serotonin, neural markers, and memory. Front. Pharmacol., (2015)

Roth, B. L. et al. Binding of typical and atypical antipsychotic agents to 5-hydroxytryptamine-6 and 5-hydroxytryptamine-7 receptors. J Pharmacol Exp Ther 268, 1403-1410 (1994).

Salis, H. M., Mirsky, E. A. & Voigt, C. A. Automated design of synthetic ribosome binding sites to control protein expression. Nat Biotechnol 27, 946-950 (2009).

Schirmaier, F. & Philippsen, P. Identification of two genes coding for the translation elongation factor EF-1 alpha of S. cerevisiae. EMBO J. 3, 3311-3315 (1984).

Sender, R., Fuchs, S. & Milo, R. Revised Estimates for the Number of Human and Bacteria Cells in the Body. PLOS Biol. 14, e1002533 (2016).

Spohn, S. N. et al. Protective Actions of Epithelial 5-Hydroxytryptamine 4 Receptors in Normal and Inflamed Colon. Gastroenterology 151, 933-944 e933 (2016).

Tepper, S. J., Rapoport, A. M. & Sheftell, F. D. Mechanisms of action of the 5-HT1B/1D receptor agonists. Arch Neurol 59, 1084-1088 (2002).

Thompson, A. J. & Lummis, S. C. The 5-HT3 receptor as a therapeutic target. Expert Opin Ther Targets 11, 527-540 (2007).

Turner, E. H., Loftis, J. M. & Blackwell, A. D. Serotonin a la carte: Supplementation with the serotonin precursor 5-hydroxytryptophan. Pharmacology and Therapeutics 109, 325-338 (2006).

Van Rensburg, P., Van Zyl, W. H. & Pretorius, I. S. Engineering yeast for efficient cellulose degradation. Yeast 14, 67-76 (1998).

Zou et al., 1,520 reference genomes from cultivated human gut bacteria enable functional microbiome analyses. Nature Biotechnology 37, 179-185 (2019)

1. A composition for use as a medicament, wherein said composition comprises cells of a recombinant microorganism, and wherein said microorganism comprises one or more recombinant nucleic acid molecules encoding one or more proteins selected from:
(a) tryptophan 5-hydroxylase (EC 1.14.16.4)
(b) tryptophan decarboxylase (EC 4.1.1.28) and
(c) tryptamine 5-hydroxylase (EC:1.14.-.-).

2. The composition for use according to claim 1, wherein said microorganism is devoid of genes capable of expressing:
i. Trp operon repressor protein, and/or
ii. Tryptophanase (EC 4.1.99.1).

3. The composition for use according to claim 1, wherein said microorganism comprises recombinant nucleic acid molecules encoding:
(a) tryptophan 5-hydroxylase (EC 1.14.16.4), and tryptophan decarboxylase (EC 4.1.1.28), or
(b) tryptophan decarboxylase (EC 4.1.1.28) and tryptamine 5-hydroxylase (EC:1.14.-.-), and
further comprises recombinant nucleic acid molecules encoding:
(c) serotonin acetyltransferase (EC 2.3.1.87) and
(d) acetylserotonin O-methyltransferase (EC 2.1.1.4),
wherein the cells are capable of producing melatonin.

4. The composition for use according to claim 1, wherein said microorganism comprises a nucleic acid molecule encoding a mutant GTP cyclohydrolase I (EC 3.5.4.16), wherein the mutant provides for an increased hydroxylation activity of said tryptophan 5-hydroxylase of at least 2-fold as compared to non-mutant GCH1, preferably a mutant having an amino acid sequence having at least about 80% sequence identity to SEQ ID No.: 2, and comprises a mutation selected from T198I, T198S, F214S, V179A, M99I and L200P.

5. The composition for use according to claim 1, wherein said microorganism comprises nucleic acid molecules encoding:
(a) tryptophan 5-hydroxylase (EC 1.14.16.4), and
(b) tryptophan decarboxylase (EC 4.1.1.28),
wherein said nucleic acid molecules encoding tryptophan 5-hydroxylase and tryptophan decarboxylase are functionally linked to a first ribosomal binding site and a second ribosomal binding site respectively; wherein the translation initiation strength of said first ribosomal binding site is at least ten fold greater than said second ribosomal binding site.

6. The composition for use according to claim 1, wherein said microorganism comprises nucleic acid molecules encoding:
(a) tryptophan 5-hydroxylase (EC 1.14.16.4), and
(b) tryptophan decarboxylase (EC 4.1.1.28),
wherein said nucleic acid molecules encoding tryptophan 5-hydroxylase and tryptophan decarboxylase are functionally linked to a first ribosomal binding site and a second ribosomal binding site respectively; wherein the translation initiation strength of said first ribosomal binding site is at least two-fold lower than said second ribosomal binding site.

7. The composition for use according to claim 1, wherein said microorganism is selected from among Escherichia, Bacteroides, Clostridium, Feacalibacterium, Eubacterium, Ruminococcus, Peptococcus, Peptostreptococcus, Lactobacillus, Lactococcus, Bifidobacterium, Enterococcus, Streptococcus, Pediococcus, Leuconostoc, Staphylococcus and Bacillus.

8. The composition for use according to claim 1, for use in preventing and/or treating TRM-; 5-HTP-; 5-HT-; or melatonin-related disorders of the central nerve system (CNS); enteric nervous system (ENS); gastro intestine (GI); hormonal imbalance, metabolic disease, non-alcoholic fatty liver disease, non-alcoholic steatohepatitis, diabetes in an animal.

9. The composition for use according to claim 8, wherein said disorder of the central nerve system is selected from group consisting of anxiety- and depression-related behavior, memory-, cognition-, and psychiatric-disorders; generalized anxiety disorder, phobia disorder, social anxiety disorder, panic disorder, obsessive-compulsive disorder, post-traumatic stress disorder, chronic stress disorder, separation and situational anxiety, age-related memory decline, dementia and disorders in sleep, spatial memory formation, alertness, focus, learning, and cognition; autism and migraine; and wherein said disorder of the gastro intestine is selected from the group consisting of immune-associated and inflammation-associated sickness; inflammatory bowel disease, irritable bowel syndrome (IBS); celiac disease, diverticular disease, and colorectal cancer.

10. The composition for use according to claim 1, wherein said composition is for oral administration to a mammal in need thereof.

11. A recombinant bacterial cell comprising one or more recombinant nucleic acid molecules or transgenes encoding one of more proteins selected from:
(a) tryptophan 5-hydroxylase (EC 1.14.16.4),
(b) tryptophan decarboxylase (EC 4.1.1.28), and
(c) tryptamine 5-hydroxylase (EC 1.14.-.-)
wherein said bacterium is devoid of genes capable of expressing:
i. trp operon repressor protein and
ii. tryptophanase (EC:4.1.99.1).

12. The recombinant bacterial cell according to claim 11, wherein the bacterial cell is selected from among Escherichia, Bacteroides, Clostridium, Feacalibacterium, Eubacterium, Ruminococcus, Peptococcus, Peptostreptococcus, Lactobacillus, Lactococcus, Bifidobacterium, Enterococcus, Streptococcus, Pediococcus, Leuconostoc, Staphylococcus and Bacillus.

13. The recombinant bacterial cell according to claim 11, wherein the amino acid sequence of said tryptophan 5-hydroxylase has at least 80% sequence identity to SEQ ID No.: 6 or 12, and wherein the amino acid sequence of said tryptophan decarboxylase has at least 80% sequence identity to SEQ ID No.: 18.

14. The recombinant bacterial cell according to claim 11, wherein said bacterium comprises nucleic acid molecules encoding:
(a) tryptophan 5-hydroxylase (EC 1.14.16.4), and
(b) tryptophan decarboxylase (EC 4.1.1.28),
wherein said nucleic acid molecules encoding tryptophan 5-hydroxylase and tryptophan decarboxylase are functionally linked to a first ribosomal binding site and a second ribosomal binding site respectively;
wherein the translation initiation strength of said first ribosomal binding site is at least ten fold greater than said second ribosomal binding site.

15. The recombinant bacterial cell according to claim 11, wherein said bacterium comprises nucleic acid molecules encoding:
(a) tryptophan 5-hydroxylase (EC 1.14.16.4), and
(b) tryptophan decarboxylase (EC 4.1.1.28),
wherein said nucleic acid molecules encoding tryptophan 5-hydroxylase and tryptophan decarboxylase are functionally linked to a first ribosomal binding site and a second ribosomal binding site respectively,
wherein the translation initiation strength of said first ribosomal binding site is at least two-fold lower than said second ribosomal binding site.

16. The recombinant bacterial cell according to claim 11 comprising recombinant nucleic acid molecules encoding:
(a) tryptophan 5-hydroxylase (EC 1.14.16.4), and tryptophan decarboxylase (EC 4.1.1.28), or
(b) tryptophan decarboxylase (EC 4.1.1.28) and tryptamine 5-hydroxylase (EC:1.14.-.-), and
further comprises recombinant nucleic acid molecules encoding:
(c) serotonin acetyltransferase (EC 2.3.1.87) and
(d) acetylserotonin O-methyltransferase (EC 2.1.1.4), wherein the cells are capable of producing melatonin.

17. The recombinant bacterial cell according to claim 11, wherein said bacterium comprises a nucleic acid sequence encoding a mutant GTP cyclohydrolase I (EC 3.5.4.16), wherein the mutant provides for an increased hydroxylation activity of said tryptophan 5-hydroxylase of at least 2-fold as compared to non-mutant GCH1.

18. The recombinant bacterial cell according to claim 17, wherein the amino acid sequence of said mutant GTP cyclohydrolase I (EC 3.5.4.16) has least about 80% sequence identity to SEQ ID No.: 2, and comprises a substitution selected from T198I, T198S, F214S, V179A, M99I and L200P.

19. The recombinant bacterial cell according to claim 11, wherein the bacterial cell is not antibiotic resistant to one or more clinically used antibiotic agents.

20. A method of treating and/or preventing a TRM-; 5-HTP-; 5-HT-; or melatonin-related disorder in a subject, the method comprising administering to the subject diagnosed with a TRM-; 5-HTP-; 5-HT-; or melatonin-related disorder recombinant bacteria engineered to express one or more
(a) tryptophan 5-hydroxylase (EC 1.14.16.4),
(b) tryptophan decarboxylase (EC 4.1.1.28), or
(c) tryptamine 5-hydroxylase (EC 1.14.-.-).

21. The method according to claim 20, wherein said microorganism is devoid of genes capable of expressing:
a. a protein that functions as a Trp operon repressor protein, and/or
b. tryptophanase (EC 4.1.99.1).

22. The method according to claim 20, wherein said microorganism comprises recombinant nucleic acid molecules encoding:
a. tryptophan 5-hydroxylase (EC 1.14.16.4), and tryptophan decarboxylase (EC 4.1.1.28), or
b. tryptophan decarboxylase (EC 4.1.1.28) and tryptamine 5-hydroxylase (EC:1.14.-.-), and further comprises recombinant nucleic acid molecules encoding:
c. serotonin acetyltransferase (EC 2.3.1.87), and
d. acetylserotonin O-methyltransferase (EC 2.1.1.4),
and wherein the cells are capable of producing melatonin.

23. The method according to claim 20, wherein said microorganism comprises a nucleic acid molecule encoding a mutant GTP cyclohydrolase I (EC 3.5.4.16), wherein the mutant provides for an increased hydroxylation activity of said tryptophan 5-hydroxylase of at least 2-fold as compared to non-mutant GCH1.

24. The method according to claim 23, wherein the mutant GTP cyclohydrolase I (EC 3.5.4.16), wherein the amino acid sequence of the mutant GTP cyclohydrolase I has at least 80% sequence identity to SEQ ID No.: 2, and comprises a mutation selected from T198I, T198S, F214S, V179A, M99I and L200P.

25. The method according to claim 20, wherein said microorganism comprises nucleic acid molecules encoding:
a. tryptophan 5-hydroxylase (EC 1.14.16.4), and
b. tryptophan decarboxylase (EC 4.1.1.28),
wherein said nucleic acid molecules encoding tryptophan 5-hydroxylase and tryptophan decarboxylase are functionally linked to a first ribosomal binding site and a second ribosomal binding site respectively; wherein the translation initiation strength of said first ribosomal binding site is at least ten fold greater than said second ribosomal binding site.

26. The method according to claim 20, wherein said microorganism comprises nucleic acid molecules encoding:
a. tryptophan 5-hydroxylase (EC 1.14.16.4), and
b. tryptophan decarboxylase (EC 4.1.1.28),
wherein said nucleic acid molecules encoding tryptophan 5-hydroxylase and tryptophan decarboxylase are functionally linked to a first ribosomal binding site and a second ribosomal binding site respectively; wherein the translation initiation strength of said first ribosomal binding site is at least two-fold lower than said second ribosomal binding site.

27. The method according to claim 20, wherein said microorganism is selected from among Escherichia, Bacteroides, Clostridium, Feacalibacterium, Eubacterium, Ruminococcus, Peptococcus, Peptostreptococcus, Lactobacillus, Lactococcus, Bifidobacterium, Enterococcus, Streptococcus, Pediococcus, Leuconostoc, Staphylococcus and Bacillus.

28. The method according to claim 20, wherein the subject is a human.

29. The method according to claim 28, wherein the TRM-; 5-HTP-; 5-HT-; or melatonin-related disorder is a disorder of the central nerve system (CNS); enteric nervous system (ENS); gastro intestine (GI); hormonal imbalance, metabolic disease, non-alcoholic fatty liver disease, non-alcoholic steatohepatitis, or diabetes.

30. The method according to claim 29, wherein the disorder of the central nerve system is selected from group consisting of anxiety- and depression-related behavior, memory-, cognition-, and psychiatric-disorders; generalized anxiety disorder, phobia disorder, social anxiety disorder, panic disorder, obsessive-compulsive disorder, post-traumatic stress disorder, chronic stress disorder, separation and situational anxiety, age-related memory decline, dementia and disorders in sleep, spatial memory formation, alertness, focus, learning, and cognition; autism and migraine; and wherein said disorder of the gastro intestine is selected from the group consisting of immune-associated and inflammation-associated sickness; inflammatory bowel disease, irritable bowel syndrome (IBS); celiac disease, diverticular disease, and colorectal cancer.


SEQUENCE LISTING (112 sequences)

SEQ ID NO: 1 - DNA, 669 bp, Escherichia coli Nissle 1917
  CDS (1)..(669): Nissle_genome_folE
atgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtcatcggtctgtcaaaaattaaccgtattgtgcagttctttgcccagcgtccgcaggtacaagaacgtctgacgcagcaaattctcattgcgctacaaacgctgctgggcaccaataacgtggctgtctctatcgacgcggtgcattactgcgtgaaggcgcgtggcatccgcgatgcaaccagtgccacgacaacgacctctcttggtggattgttcaaatccagtcagaatacgcgccacgagtttctgcgcgctgtgcgtcatcacaactga

SEQ ID NO: 2 - protein, 222 aa, Escherichia coli Nissle 1917
MPSLSKEAALVHEALVARGLETPLRPPVHEMDNETRKSLIAGHMTEIMQLLNLDLADDSLMETPHRIAKMYVDEIFSGLDYANFPKITLIENKMKVDEMVTVRDITLTSTCEHHFVTIDGKATVAYIPKDSVIGLSKINRIVQFFAQRPQVQERLTQQILIALQTLLGTNNVAVSIDAVHYCVKARGIRDATSATTTTSLGGLFKSSQNTRHEFLRAVRHHN

SEQ ID NO: 3 - DNA, 669 bp, Escherichia coli Nissle 1917
  CDS (1)..(669): mutated folE(T198I)
atgccatcactcagtaaagaagcggccctggttcatgaagcgttagttgcgcgaggactggaaacaccgctgcgcccgcccgtgcatgaaatggataacgaaacgcgcaaaagccttattgctggtcatatgaccgaaatcatgcagctgctgaatctcgacctggctgatgacagtttgatggaaacgccgcatcgcatcgctaaaatgtatgtcgatgaaattttctccggtctggattacgccaatttcccgaaaatcaccctcattgaaaacaaaatgaaggtcgatgaaatggtcaccgtgcgcgatatcactctgaccagcacctgtgaacaccattttgttaccatcgatggcaaagcgacggtggcctatatcccgaaagattcggtcatcggtctgtcaaaaattaaccgtattgtgcagttctttgcccagcgtccgcaggtacaagaacgtctgacgcagcaaattctcattgcgctacaaacgctgctgggcaccaataacgtggctgtctctatcgacgcggtgcattactgcgtgaaggcgcgtggcatccgcgatgcaaccagtgccacgacaacgatctctcttggtggattgttcaaatccagtcagaatacgcgccacgagtttctgcgcgctgtgcgtcatcacaactga

SEQ ID NO: 4 - protein, 222 aa, Escherichia coli Nissle 1917
MPSLSKEAALVHEALVARGLETPLRPPVHEMDNETRKSLIAGHMTEIMQLLNLDLADDSLMETPHRIAKMYVDEIFSGLDYANFPKITLIENKMKVDEMVTVRDITLTSTCEHHFVTIDGKATVAYIPKDSVIGLSKINRIVQFFAQRPQVQERLTQQILIALQTLLGTNNVAVSIDAVHYCVKARGIRDATSATTTISLGGLFKSSQNTRHEFLRAVRHHN

SEQ ID NO: 5 - DNA, 948 bp, mouse
  CDS (1)..(948): mouse TPH1 tryptophan 5-hydroxylase (EC 1.14.16.4)
atggagactgtgccctggttcccaaagaaaatctcggatctggatttctgtgctaaccgcgtgttattatacggatcagaattggatgcagaccatccagggtttaaggataatgtttaccgtcgtcgccgcaagtatttcgcagaattagcgatgaactataaacacggtgaccctattcctaagattgaattcacggaggaagagattaaaacttggggtaccatttttcgcgagctgaataagttatatccaacacatgcctgtcgcgagtatttacgtaaccttccccttttgagtaagtattgtgggtatcgcgaagacaacatccctcagctggaagacgtaagtaacttcttgaaggaacgtactggattctcgatccgcccggttgccggttatttatcaccccgcgattttttaagtggccttgcgttccgcgtgtttcattgcactcagtacgtccgccactcgtcagatccgctttatacacctgagccagatacatgccacgaactgttaggtcatgtgccattacttgcagaacctagcttcgcacagtttagtcaagagatcgggcttgcgtcattaggcgcatcggaagaaaccgtacaaaaattggcgacttgctattttttcactgttgagttcggtctgtgtaagcaggacggccaactgcgcgtgtttggggcgggcctgctgtccagtattagtgaattaaaacatgctctgagtggtcacgccaaagtgaagccctttgaccctaagatcgcttgcaaacaggaatgtttgattacgtcgttccaagacgtatacttcgtctctgagtcgtttgaggatgcgaaggagaaaatgcgcgagtttgctaaaacagtgaaacgccctttcggcctgaagtataacccttacactcagtccgtgcaggtgttgcgtgatacgaaaagctaa

SEQ ID NO: 6 - protein, 315 aa, mouse
METVPWFPKKISDLDFCANRVLLYGSELDADHPGFKDNVYRRRRKYFAELAMNYKHGDPIPKIEFTEEEIKTWGTIFRELNKLYPTHACREYLRNLPLLSKYCGYREDNIPQLEDVSNFLKERTGFSIRPVAGYLSPRDFLSGLAFRVFHCTQYVRHSSDPLYTPEPDTCHELLGHVPLLAEPSFAQFSQEIGLASLGASEETVQKLATCYFFTVEFGLCKQDGQLRVFGAGLLSSISELKHALSGHAKVKPFDPKIACKQECLITSFQDVYFVSESFEDAKEKMREFAKTVKRPFGLKYNPYTQSVQVLRDTKS

SEQ ID NO: 7 - DNA, 963 bp, mouse
  CDS (1)..(963): mouse TPH2 tryptophan 5-hydroxylase (EC 1.14.16.4)
atggaagaggaggatcttgaagacgtaccttggttcccccgtaagatttcggaattggatcgttgcagccaccgcgtgctgatgtacggcacggagttggacgcggatcatccgggctttaaagacaacgtctaccgtcagcgtcgtaagtattttgttgacgtcgctatggggtataaatacggtcagccaattccccgcgttgaatatacagaggaagaaaccaaaacatggggcgtagtctttcgcgagcttagcaagctgtaccctacccatgcttgccgtgagtatttgaagaatttaccgttactgactaaatattgcggataccgtgaggacaatgttccgcagttagaggacgtgagcatgttcttaaaggaacgcagtggcttcacggtgcgccctgttgcgggctatttatccccacgtgatttcttggcaggcttggcataccgtgtcttccactgcacacaatacgtgcgccacggatcagaccctttgtatacccccgaaccggatacttgccacgagctgctggggcatgtaccacttttagctgacccaaagtttgcacaattttcgcaagagatcggcttagcctccttaggcgcatcagatgaggatgtccagaagcttgccacctgttattttttcaccatcgagtttgggctttgcaagcaggagggacagttgcgcgcatacggggcagggcttttgtcctccatcggggaattaaagcatgcgttaagtgacaaagcgtgtgtcaagtcatttgatcccaaaaccacgtgtttacaggaatgcttaattacgacgttccaggatgcttattttgtcagtgatagcttcgaggaagccaaagaaaaaatgcgcgacttcgctaaatccattacgcgcccgttttctgtctattttaacccttacacgcaatccattgagatcttaaaagacactcgcagctaa

SEQ ID NO: 8 - protein, 320 aa, mouse
MEEEDLEDVPWFPRKISELDRCSHRVLMYGTELDADHPGFKDNVYRQRRKYFVDVAMGYKYGQPIPRVEYTEEETKTWGVVFRELSKLYPTHACREYLKNLPLLTKYCGYREDNVPQLEDVSMFLKERSGFTVRPVAGYLSPRDFLAGLAYRVFHCTQYVRHGSDPLYTPEPDTCHELLGHVPLLADPKFAQFSQEIGLASLGASDEDVQKLATCYFFTIEFGLCKQEGQLRAYGAGLLSSIGELKHALSDKACVKSFDPKTTCLQECLITTFQDAYFVSDSFEEAKEKMRDFAKSITRPFSVYFNPYTQSIEILKDTRS

SEQ ID NO: 9 - DNA, 948 bp, human
  CDS (1)..(948): human TPH1 tryptophan 5-hydroxylase
atggagaccgttccgtggttccctaaaaagatctcggaccttgatcactgcgctaatcgtgttctgatgtacggatcagagcttgacgcggaccaccctggtttcaaagacaacgtctaccgtaaacgccgcaagtatttcgctgatctggctatgaactacaaacatggagatccaatcccaaaggtcgaatttacagaggaagaaattaaaacctggggcaccgtgttccaagaattgaataagttgtaccctacgcacgcatgccgcgaatatttgaaaaaccttccacttctgtccaaatattgtggctatcgtgaagataatattccacaattagaggatgtcagcaattttttgaaagaacgcactgggttttcaattcgtccggtcgctggatatctgagcccgcgtgacttcctttcggggctggctttccgtgtcttccactgtactcaatacgtccgccattcgtcggaccctttttacactcctgagccggatacttgccacgaattattggggcatgttccgcttctggccgagccgtcgtttgctcagtttagtcaagagatcggtttggcgagtttgggtgcatcggaagaggctgtccaaaagttagccacttgttacttttttaccgtcgaatttggcttatgcaagcaagacggacagcttcgtgtctttggcgccgggttgttgtctagtatcagtgaattgaagcacgccctgtccggacatgccaaggtaaagcctttcgatccaaagattacctgcaagcaagagtgtttgattaccacgttccaggatgtctacttcgtctccgagtcgtttgaggacgcaaaggagaagatgcgtgaatttaccaaaacgatcaaacgtcccttcggggtaaagtacaacccttatacacgctccatccagattcttaaagacactaagtcgtaa

SEQ ID NO: 10 - protein, 315 aa, human
METVPWFPKKISDLDHCANRVLMYGSELDADHPGFKDNVYRKRRKYFADLAMNYKHGDPIPKVEFTEEEIKTWGTVFQELNKLYPTHACREYLKNLPLLSKYCGYREDNIPQLEDVSNFLKERTGFSIRPVAGYLSPRDFLSGLAFRVFHCTQYVRHSSDPFYTPEPDTCHELLGHVPLLAEPSFAQFSQEIGLASLGASEEAVQKLATCYFFTVEFGLCKQDGQLRVFGAGLLSSISELKHALSGHAKVKPFDPKITCKQECLITTFQDVYFVSESFEDAKEKMREFTKTIKRPFGVKYNPYTRSIQILKDTKS

SEQ ID NO: 11 - DNA, 963 bp, human
  CDS (1)..(963): human TPH2 tryptophan 5-hydroxylase
atggaagaagaagaattagaggacgttccgtggtttccgcgtaaaattagcgaactggataaatgtagccatcgtgttctgatgtatggtagtgaactggatgcagatcatccgggttttaaagataatgtttatcgtcagcgtcgcaagtattttgttgatgttgcaatgggttacaaatacggtcagccgattccgcgtgttgaatataccgaagaagaaaccaaaacctggggtgttgtttttcgtgaactgagcaaactgtatccgacacatgcctgtcgtgaatatctgaaaaactttccgctgctgaccaaatattgtggttatcgtgaagataacgttccgcagctggaagatgttagcatgtttctgaaagaacgtagcggttttaccgttcgtccggttgcaggttatctgagtccgcgtgattttctggcaggtctggcatatcgtgtttttcattgtacccagtatattcgccatggtagcgatccgctgtatactccggaaccggatacctgtcatgaactgctgggtcatgtgccgctgctggcagatccgaaatttgcacagtttagccaagaaattggtctggcaagcctgggtgcaagtgatgaagatgttcagaaactggcaacctgctattttttcaccattgaatttggcctgtgcaaacaagagggtcagctgcgtgcatatggtgcaggtctgctgagcagcattggtgaactgaaacatgcactgagcgataaagcatgtgttaaagcatttgatccgaaaaccacctgtctgcaagaatgtctgattaccacctttcaagaagcctatttcgttagcgaaagctttgaagaggccaaagaaaaaatgcgcgattttgccaaaagcattacccgtccgtttagcgtttatttcaatccgtatacacagagcatcgagatcctgaaagatacccgtagctaa

SEQ ID NO: 12 - protein, 320 aa, human
MEEEELEDVPWFPRKISELDKCSHRVLMYGSELDADHPGFKDNVYRQRRKYFVDVAMGYKYGQPIPRVEYTEEETKTWGVVFRELSKLYPTHACREYLKNFPLLTKYCGYREDNVPQLEDVSMFLKERSGFTVRPVAGYLSPRDFLAGLAYRVFHCTQYIRHGSDPLYTPEPDTCHELLGHVPLLADPKFAQFSQEIGLASLGASDEDVQKLATCYFFTIEFGLCKQEGQLRAYGAGLLSSIGELKHALSDKACVKAFDPKTTCLQECLITTFQEAYFVSESFEEAKEKMRDFAKSITRPFSVYFNPYTQSIEILKDTRS

SEQ ID NO: 13 - DNA, 1503 bp, Catharanthus roseus
  CDS (1)..(1503): Madagascar periwinkle TDC tryptophan decarboxylase (EC 4.1.1.28)
atgggatccatcgattcaacaaatgttgccatgtcaaactcgcccgtcggggagttcaaacctctggaagcggaggaattccgtaaacaagcccaccgtatggttgacttcatcgcagactactataagaatgttgagacatatcccgtccttagtgaggtcgaacctggatacctgcgtaagcgcatccccgagacggcaccttacttacccgagccgttagacgacattatgaaggacattcaaaaggacatcatcccgggcatgacaaattggatgtccccgaacttctatgctttctttccagcgactgtatcctcagcagcttttttaggtgagatgttatctacagcgctgaactcagttggatttacgtgggtttcatcacctgcggctacggagttggagatgatcgtcatggattggttagcccaaatcttaaaattgcctaagtcattcatgttttccgggaccggcggcggagtgattcagaacacgacctccgagtcaattctttgcactatcatcgcggcccgtgagcgcgctcttgaaaagttgggcccagattctatcggtaagttggtatgctatgggagcgatcaaacgcacaccatgttcccgaaaacctgtaagttggcaggtatctaccctaacaatattcgcttaatccccactactgtagagactgatttcggtatttcgcctcaggtgttgcgtaaaatggtagaagacgacgtggctgccgggtatgtgccgctgttcctgtgcgctacattaggaacaacctctactacagccaccgatccggtcgacagtttatcagagattgcgaatgagtttggaatctggattcatgttgatgcagcctatgccggctcagcgtgcatctgcccagagtttcgccattatctggacggaatcgagcgtgtcgacagcctttcccttagcccgcataaatggttattggcgtacttagattgcacgtgcttatgggttaagcagcctcatctgcttttgcgtgcattaacaactaacccggaatatctgaaaaacaagcagtcggatttagataaggtagttgattttaagaattggcaaattgccactgggcgtaaattccgttcccttaagttatggctgatccttcgctcctacggagtagttaaccttcaaagtcacattcgctcagacgttgcaatggggaaaatgtttgaagaatgggtacgctcagactcccgcttcgaaatcgtagtcccacgtaatttcagcttggtatgctttcgcctgaagcccgatgtaagttcacttcacgtggaggaggttaacaaaaagctgcttgatatgctgaattcaaccggacgcgtatacatgacgcatacaatcgtgggagggatttatatgttgcgcttagcagtaggctccagcttgacagaagaacatcatgtgcgtcgcgtgtgggaccttatccaaaaattgacagacgatttattgaaagaggcataa

SEQ ID NO: 14 - protein, 500 aa, Catharanthus roseus
MGSIDSTNVAMSNSPVGEFKPLEAEEFRKQAHRMVDFIADYYKNVETYPVLSEVEPGYLRKRIPETAPYLPEPLDDIMKDIQKDIIPGMTNWMSPNFYAFFPATVSSAAFLGEMLSTALNSVGFTWVSSPAATELEMIVMDWLAQILKLPKSFMFSGTGGGVIQNTTSESILCTIIAARERALEKLGPDSIGKLVCYGSDQTHTMFPKTCKLAGIYPNNIRLIPTTVETDFGISPQVLRKMVEDDVAAGYVPLFLCATLGTTSTTATDPVDSLSEIANEFGIWIHVDAAYAGSACICPEFRHYLDGIERVDSLSLSPHKWLLAYLDCTCLWVKQPHLLLRALTTNPEYLKNKQSDLDKVVDFKNWQIATGRKFRSLKLWLILRSYGVVNLQSHIRSDVAMGKMFEEWVRSDSRFEIVVPRNFSLVCFRLKPDVSSLHVEEVNKKLLDMLNSTGRVYMTHTIVGGIYMLRLAVGSSLTEEHHVRRVWDLIQKLTDDLLKEA

SEQ ID NO: 15 - DNA, 1443 bp, Cavia porcellus
  CDS (1)..(1443): guinea pig TDC tryptophan decarboxylase (EC 4.1.1.28)
atgaacgcatctgagtttcgtcgtcgtgggaaggaaatggtagactacgtagctaactaccttgagggcatcgagtcacgcctggtctatccggatgttgagcctggatacttacgtccgctgattcctagcagcgcacccgaagaacccgagacgtatgaggacattattggagatattgaacgtatcatcatgcctggcgtaacgcattggaacagcccgtacttctttgcgtactttcctactgctaattcttacccgtcaatgttagcagatatgttgtgtggtgcgatctcatgtatcggcttttcctgggcggcgagccctgcttgcacagagctggagacagtcatgcttgactggttgggaaaaatgttgcgcttacctgatgcatttcttgcgggtaacgcaggcatgggaggaggggtgattcagggaagtgcctccgaagctaccctggtcgcacttttagctgcccgcacaaaggtaattcgccgtctgcaagcagcatcgccagaactgacccaggcagccatcatggaaaagttagtggcctacgcttcggatcaggctcactcttctgtcgagcgcgctggcttaattggcggagttcgtatgaagctgattccttcagatagcaactttgccatgcgtgctagtgctctgcgcgaagctttggagcgtgataaagcggcaggcttaattccgtttttcgttgtggcaacacttggcaccactaattgctgttcatttgattcgttgttagaagtaggtccaatttgcaaccaagaggaaatgtggttgcatattgatgcagcgtacgctggctcggcttttatttgccctgaattccgccacttattggatggggtggagtttgccgacagtttcaatttcaacccccacaaatggctgttggttaatttcgattgcagcgcgatgtgggtaaaacaacgtacggatttaatcggcgctttcaaacttgaccctgtttatcttaagcatggccatcaagacagtggtttgatcacagactatcgccactggcaaattcccctgggtcgccgttttcgcagtttgaagatgtggtttgtattccgcatgtatgggatcaagggtctgcaggcacatatccgcaagcatgttcaacttgcgcatgaatttgagagtcttgtacgccaggacccgcgttttgaaatctgcatggaagttaccttgggactggtttgctttcgtcttaagggtagcaatcagttaaatgagactttgcttaaacgtatcaactcggctcgtaagattcaccttgttccctgccacttacgtgataaatttgttctgcgctttcgtatctgctcacgccaagtggagtcagaccatgtgcaacaggcttggcaacatattcgccaacttgcgtcgagtgtacttcgtttagaacgcgcttaa

SEQ ID NO: 16 - protein, 480 aa, Cavia porcellus
MNASEFRRRGKEMVDYVANYLEGIESRLVYPDVEPGYLRPLIPSSAPEEPETYEDIIGDIERIIMPGVTHWNSPYFFAYFPTANSYPSMLADMLCGAISCIGFSWAASPACTELETVMLDWLGKMLRLPDAFLAGNAGMGGGVIQGSASEATLVALLAARTKVIRRLQAASPELTQAAIMEKLVAYASDQAHSSVERAGLIGGVRMKLIPSDSNFAMRASALREALERDKAAGLIPFFVVATLGTTNCCSFDSLLEVGPICNQEEMWLHIDAAYAGSAFICPEFRHLLDGVEFADSFNFNPHKWLLVNFDCSAMWVKQRTDLIGAFKLDPVYLKHGHQDSGLITDYRHWQIPLGRRFRSLKMWFVFRMYGIKGLQAHIRKHVQLAHEFESLVRQDPRFEICMEVTLGLVCFRLKGSNQLNETLLKRINSARKIHLVPCHLRDKFVLRFRICSRQVESDHVQQAWQHIRQLASSVLRLERA

SEQ ID NO: 17 - DNA, 1545 bp, Ozyza sativa subsp. japonica
  CDS (1)..(1545): rice TDC tryptophan decarboxylase (EC 4.1.1.28)
atgggtagcctggataccaatccgaccgcatttagcgcatttccggcaggcgaaggtgaaacctttcagccgctgaatgcagatgatgttcgtagctatctgcataaagccgttgattttatcagcgactattacaaaagcgttgaaagcatgccggttctgccgaatgttaaaccgggttatctgcaggatgaactgcgtgcaagccctccgacctatagcgcaccgtttgatgttaccatgaaagaactgcgtagcagcgttgttccgggtatgacccattgggcaagcccgaacttttttgcattttttccgagcaccaatagcgcagcagcaattgccggtgatctgattgcaagcgcaatgaataccgttggttttacctggcaggcaagtccggcagcaaccgaaatggaagttctggcactggattggctggcacagatgctgaatctgccgaccagctttatgaatcgtaccggtgaaggtcgtggcaccggtggtggtgttattctgggtacaaccagcgaagcaatgctggttaccctggttgcagcacgtgatgcagcactgcgtcgtagcggtagtgatggtgttgcaggtctgcatcgtctggcagtttatgcagcagatcagacccatagcacctttttcaaagcctgtcgcctggcaggttttgatccggcaaatattcgtagcattccgaccggtgcagaaaccgattatggtctggatccggcacgtctgctggaagccatgcaggcagatgcagatgccggtctggtgccgacctatgtttgtgcaaccgttggcaccaccagtagcaatgcagttgatccggttggtgcagttgcagatgttgcagcccgttttgcagcatgggttcatgttgatgcagcctatgcaggtagcgcatgtatttgtccggaatttcgtcatcatctggatggtgtggaacgtgttgatagcattagcatgagtccgcataaatggctgatgacctgtctggattgtacctgtctgtatgttcgtgatacccatcgtctgaccggttcactggaaacaaatccggaatatctgaaaaatcatgcaagcgatagcggtgaagttaccgatctgaaagatatgcaggttggtgttggtcgtcgttttcgtggtctgaaactgtggatggttatgcgtacctatggtgtggcaaaactgcaagaacatattcgttctgatgttgcaatggccaaagtgtttgaagatctggttcgtggtgatgatcgttttgaagttgttgttccgcgtaattttgccctggtttgttttcgtattcgtgccggtgcgggtgcagcagcagccaccgaagaagatgcagacgaagcaaatcgtgaactgatggaacgtctgaataaaaccggtaaagcatatgttgcacataccgttgttggtggtcgctttgttctgcgttttgcggttggtagcagcctgcaagaagaacatcacgttcgtagcgcctgggaactgatcaaaaaaaccaccacagaaatgatgaattaa

SEQ ID NO: 18 - protein, 514 aa, Ozyza sativa subsp. japonica
MGSLDTNPTAFSAFPAGEGETFQPLNADDVRSYLHKAVDFISDYYKSVESMPVLPNVKPGYLQDELRASPPTYSAPFDVTMKELRSSVVPGMTHWASPNFFAFFPSTNSAAAIAGDLIASAMNTVGFTWQASPAATEMEVLALDWLAQMLNLPTSFMNRTGEGRGTGGGVILGTTSEAMLVTLVAARDAALRRSGSDGVAGLHRLAVYAADQTHSTFFKACRLAGFDPANIRSIPTGAETDYGLDPARLLEAMQADADAGLVPTYVCATVGTTSSNAVDPVGAVADVAARFAAWVHVDAAYAGSACICPEFRHHLDGVERVDSISMSPHKWLMTCLDCTCLYVRDTHRLTGSLETNPEYLKNHASDSGEVTDLKDMQVGVGRRFRGLKLWMVMRTYGVAKLQEHIRSDVAMAKVFEDLVRGDDRFEVVVPRNFALVCFRIRAGAGAAAATEEDADEANRELMERLNKTGKAYVAHTVVGGRFVLRFAVGSSLQEEHHVRSAWELIKKTTTEMMN

SEQ ID NO: 19 - DNA, 1569 bp, Artificial Sequence
  Synthetic
  CDS (1)..(1569): T5H - Tryptamine 5-hydroxylase nucleotide Q2QUC5 codon optimized
atggaattaaccatggcatcaaccatgtccctggccttacttgtattaagcgccgcatacgtgctggtcgccttgcgccgttcccgttcgtctagtagcaaaccccgccgccttccacccagtcctccaggatggccggtgatcggtcatctgcacttgatgtcaggcatgccccatcacgcattagccgaactggcacgcaccatgcgtgctcctcttttccgcatgcgtctgggatctgtgcctgcagtcgtaatttccaaacccgaccttgcccgtgctgcgctgaccaccaacgatgctgctttggcttcacgtccccaccttttgtcaggacagttcttgtcattcggatgctcggacgtgacctttgcaccagctggcccataccaccgtatggcacgtcgcgtagtagtgagcgaactgttatcggcccgccgcgtagcgacatatggtgcagtgcgcgtcaaggaactgcgtcgccttttagctcaccttacaaagaatacgagtcccgcaaaaccggtcgaccttagcgaatgcttccttaatttggccaatgatgtgctgtgccgtgtagccttcggccgtcgttttcctcatggagaaggagataaacttggcgcggtactggctgaagcacaggatcttttcgcgggttttacgattggtgacttttttccggaacttgagcccgtcgcatcgactgttacgggattacgtcgtcgcttaaaaaagtgtctggctgacctgcgcgaggcgtgcgacgttattgtcgatgaacacatcagcggcaaccgccagcgcatccccggagaccgtgacgaagatttcgttgatgttcttctgcgtgtacagaaaagtcctgatttagaagtcccacttacagacgataacttgaaggcgcttgtgcttgatatgttcgtggcgggaacggacacgactttcgccaccttagagtgggtcatgaccgaattagtgcgccacccccgcatccttaagaaggcccaagaggaggttcgtcgtgttgtgggagattctggccgtgttgaagagtcccatttgggggagcttcactatatgcgcgctattatcaaggagacattccgcttgcatccggcggtaccattacttgttcctcgtgagagtgtcgccccatgtacgctggggggatatgatatcccggctcgtacccgtgtatttatcaacactttcgctatggggcgcgacccagagatttgggacaacccactggagtactccccggagcgtttcgaatcagccgggggcgggggtgaaattgacttgaaggaccccgactacaaactgttacctttcgggggtgggcgtcgcggatgcccagggtatacgttcgctttagcaacggtacaggtgtcgttggcgtctctgttgtaccattttgaatgggctttacctgctggagtacgtgccgaggatgtaaatttagatgaaaccttcggacttgcaacccgtaaaaaagaaccattgttcgtagcagtgcgcaagagcgatgcttatgaattcaagggagaggaactttcggaggtc

SEQ ID NO: 20 - protein, 523 aa, Artificial Sequence
  Synthetic Construct
MELTMASTMSLALLVLSAAYVLVALRRSRSSSSKPRRLPPSPPGWPVIGHLHLMSGMPHHALAELARTMRAPLFRMRLGSVPAVVISKPDLARAALTTNDAALASRPHLLSGQFLSFGCSDVTFAPAGPYHRMARRVVVSELLSARRVATYGAVRVKELRRLLAHLTKNTSPAKPVDLSECFLNLANDVLCRVAFGRRFPHGEGDKLGAVLAEAQDLFAGFTIGDFFPELEPVASTVTGLRRRLKKCLADLREACDVIVDEHISGNRQRIPGDRDEDFVDVLLRVQKSPDLEVPLTDDNLKALVLDMFVAGTDTTFATLEWVMTELVRHPRILKKAQEEVRRVVGDSGRVEESHLGELHYMRAIIKETFRLHPAVPLLVPRESVAPCTLGGYDIPARTRVFINTFAMGRDPEIWDNPLEYSPERFESAGGGGEIDLKDPDYKLLPFGGGRRGCPGYTFALATVQVSLASLLYHFEWALPAGVRAEDVNLDETFGLATRKKEPLFVAVRKSDAYEFKGEELSEV

SEQ ID NO: 21 - DNA, 624 bp, Homo sapiens (Human)
  CDS (1)..(624): AANAT - codon optimized for E. coli - endocing Serotonin N-acetyltransferase EC2.3.1.87
atgagtacacagtccactcatccgttgaagcccgaggcaccacgtttaccgcctggtattccagagtccccatcgtgccaacgccgccacacattaccagcatcagagttccgctgcctgacaccagaagatgcagtttccgcctttgagatcgaacgcgaagcattcatcagcgtgcttggggtttgtcccctttacctggacgaaatccgccattttctgaccctgtgccctgaacttagcttaggctggttcgaggaaggatgcttggtggcttttattattggatccttgtgggataaagaacgcttaatgcaagaaagccttacgttgcatcgctcgggaggacatatcgcgcatttgcacgtcttggcagttcatcgtgctttccgccaacaggggcgtgggccgatcttactgtggcgttacttgcatcaccttgggtctcaacctgcggttcgccgtgcagccttgatgtgtgaggacgcgttagttcccttttatgagcgtttcagttttcacgccgtcgggccgtgcgctatcaccgtaggtagcctgaccttcatggagttgcattgcagtttacgcggacatccttttttgcgtcgcaactccgggtgttga

SEQ ID NO: 22 - protein, 207 aa, Homo sapiens (Human)
MSTQSTHPLKPEAPRLPPGIPESPSCQRRHTLPASEFRCLTPEDAVSAFEIEREAFISVLGVCPLYLDEIRHFLTLCPELSLGWFEEGCLVAFIIGSLWDKERLMQESLTLHRSGGHIAHLHVLAVHRAFRQQGRGPILLWRYLHHLGSQPAVRRAALMCEDALVPFYERFSFHAVGPCAITVGSLTFMELHCSLRGHPFLRRNSGC

SEQ ID NO: 23 - DNA, 1038 bp, Homo sapiens (Human)
  CDS (1)..(1038): ASMT - codonoptimized for E. coli - encoding Acetylserotonin O-methyltransferase EC2.1.1.4
atgggcagttcggaagatcaagcctatcgcctgctgaatgattacgccaacggtttcatggtgagtcaggtattgttcgctgcgtgcgaattgggcgtttttgatctgcttgcagaagcaccgggtccgcttgatgttgctgctgtggcggctggcgtacgcgcgtccgctcacggaactgaattgttacttgatatctgtgtctctttgaagcttctgaaagtcgagacgcgcggaggtaaggcgttctaccgcaacacggagctttcaagcgattatttgaccacggtgtcgcctacgtcgcagtgcagtatgctgaaatacatgggacgtactagctatcgttgctggggacacctggcagatgcagttcgcgagggacgcaatcagtatctggaaacctttggagtacctgcggaagaattgtttacggctatctaccgctccgagggagagcgtctgcaatttatgcaagcgctgcaggaggtgtggagcgtcaacgggcgtagcgttttaacggcgttcgatctgtcagtcttcccattgatgtgtgaccttggtgggggtgcgggcgccctggcgaaggagtgtatgtcgctgtatcccggctgtaagattaccgtcttcgacattccggaggttgtttggaccgcgaaacagcatttttctttccaggaagaagaacagattgacttccaggaaggagactttttcaaagatcccttgcctgaggctgacttatacatcttagcacgcgttctgcacgactgggccgacggaaaatgttcgcatctgttggaacgtatctaccatacgtgtaagccgggaggcggcattctggtcattgagtccttactggatgaggatcgtcgcggcccccttttaacccagctttattcgcttaatatgcttgtgcagactgagggccaggagcgcacgcccacacactaccatatgctgttatcaagcgcaggttttcgcgactttcaatttaaaaagacaggcgctatctatgacgcgattttagcacgcaaataa

SEQ ID NO: 24 - protein, 345 aa, Homo sapiens (Human)
MGSSEDQAYRLLNDYANGFMVSQVLFAACELGVFDLLAEAPGPLDVAAVAAGVRASAHGTELLLDICVSLKLLKVETRGGKAFYRNTELSSDYLTTVSPTSQCSMLKYMGRTSYRCWGHLADAVREGRNQYLETFGVPAEELFTAIYRSEGERLQFMQALQEVWSVNGRSVLTAFDLSVFPLMCDLGGGAGALAKECMSLYPGCKITVFDIPEVVWTAKQHFSFQEEEQIDFQEGDFFKDPLPEADLYILARVLHDWADGKCSHLLERIYHTCKPGGGILVIESLLDEDRRGPLLTQLYSLNMLVQTEGQERTPTHYHMLLSSAGFRDFQFKKTGAIYDAILARK

SEQ ID NO: 25 - DNA, 327 bp, Escherichia coli Nissle 1917
  CDS (1)..(327): trp operon repressor
atggcccaacaatcaccctattcagcagcgatggcagaacagcgtcaccaggagtggttacgttttgtcgacctgcttaagaatgcctaccaaaacgatctccatttaccgttgttaaacctgatgctgacgccagatgagcgcgaagcgttggggactcgcgtgcgtattgtcgaagagctgttgcgcggcgaaatgagccagcgtgagttaaaaaatgaactcggcgcgggcatcgcgacgattacgcgtggatctaacagcctgaaagccgcgcccgttgagctgcgccagtggctggaagaggtgttgctgaaaagcgattga

SEQ ID NO: 26 - protein, 108 aa, Escherichia coli Nissle 1917
MAQQSPYSAAMAEQRHQEWLRFVDLLKNAYQNDLHLPLLNLMLTPDEREALGTRVRIVEELLRGEMSQRELKNELGAGIATITRGSNSLKAAPVELRQWLEEVLLKSD

SEQ ID NO: 27 - DNA, 105 bp, Escherichia coli Nissle 1917
  misc_feature (1)..(105): trpR knockout
atgattccggggatccgtcgacctgcagttcgaagttcctattctctagaaagtataggaacttcgaagcagctccagcctacagtgttgctgaaaagcgattga

SEQ ID NO: 28 - DNA, 1416 bp, Escherichia coli Nissle 1917
  CDS (1)..(1416): tnaA tryptophanase (EC 4.1.99.1)
atggaaaactttaaacatctccctgaaccgttccgcattcgtgttattgagccagtaaaacgtaccactcgcgcttatcgtgaagaggcaattattaaatccggcatgaacccgttcctgctggatagcgaagatgtgtttatcgatttactgaccgacagcggcaccggggcggtgacgcagagtatgcaggccgcgatgatgcgcggcgacgaagcttacagcggcagccgcagctactacgcgttagccgagtcagtgaaaaatatctttggttatcaatacactattccgactcaccagggccgtggtgcagagcaaatctatattccggtactgattaaaaaacgcgagcaggaaaaaggcctggatcgcagcaaaatggtggcgttctctaactatttctttgataccacgcagggccatagccagattaacggctgtaccgtgcgtaacgtctatatcaaagaagccttcgatactggcgtgcgttacgactttaaaggcaactttgaccttgagggattagaacgcggtattgaagaagttggcccgaataacgtgccgtatatcgttgcaaccatcaccagtaactccgcaggtggtcagccggtttcactggcaaacttaaaagtgatgtacagcatcgcgaagaaatacgatattccggtggtcatggactccgcacgctttgccgaaaacgcctatttcatcaagcagcgtgaagcagaatacaaagactggaccatcgagcagatcacccgcgaaacctacaaatatgccgatatgctggcgatgtccgccaagaaagatgcgatggtgccgatgggcggcctgctgtgcatgaaagacgacagcttctttgatgtgtataccgaatgcagaaccctttgcgtggtgcaggaaggcttcccgacatatggcggcctggaaggcggcgctatggagcgtctggcggtaggtctgtatgacggcatgaatctggactggctggcttatcgtatcgcacaggtgcagtatctggtcgatggtctggaagagattggcgttgtctgtcagcaggcgggcggtcacgcggcattcgttgatgcaggtaaactgttgccgcatatcccggcagatcagttcccggcacaggcgctggcctgcgagctgtataaagtcgccggtatccgtgcggtagaaattggctctttcctgttaggccgcgatccgaaaaccggtaaacaactgccatgcccggctgaactgctgcgtttaaccattccgcgcgcaacatatactcaaacacatatggacttcattattgaagcctttaaacatgtgaaagagaacgcggcgaatattaaagggttaacctttacctacgaaccgaaagtattgcgtcacttcaccgcaaaactgaaagaagtttaa

SEQ ID NO: 29 - protein, 471 aa, Escherichia coli Nissle 1917
MENFKHLPEPFRIRVIEPVKRTTRAYREEAIIKSGMNPFLLDSEDVFIDLLTDSGTGAVTQSMQAAMMRGDEAYSGSRSYYALAESVKNIFGYQYTIPTHQGRGAEQIYIPVLIKKREQEKGLDRSKMVAFSNYFFDTTQGHSQINGCTVRNVYIKEAFDTGVRYDFKGNFDLEGLERGIEEVGPNNVPYIVATITSNSAGGQPVSLANLKVMYSIAKKYDIPVVMDSARFAENAYFIKQREAEYKDWTIEQITRETYKYADMLAMSAKKDAMVPMGGLLCMKDDSFFDVYTECRTLCVVQEGFPTYGGLEGGAMERLAVGLYDGMNLDWLAYRIAQVQYLVDGLEEIGVVCQQAGGHAAFVDAGKLLPHIPADQFPAQALACELYKVAGIRAVEIGSFLLGRDPKTGKQLPCPAELLRLTIPRATYTQTHMDFIIEAFKHVKENAANIKGLTFTYEPKVLRHFTAKLKEV

SEQ ID NO: 30 - DNA, 105 bp, Escherichia coli Nissle 1917
  misc_feature (1)..(105): tnaA knockout
atgattccggggatccgtcgacctgcagttcgaagttcctattctctagaaagtataggaacttcgaagcagctccagcctacagcaaaactgaaagaagtttaa

SEQ ID NO: 31 - DNA, 219 bp, E. coli Nissle 1917
  CDS (1)..(219): infA
atggccaaagaagacaatattgaaatgcaaggtaccgttcttgaaacgttgcctaataccatgttccgcgtagagttagaaaacggtcacgtggttactgcacacatctccggtaaaatgcgcaaaaactacatccgcatcctgacgggcgacaaagtgactgttgaactgaccccgtacgacctgagcaaaggccgcattgtcttccgtagtcgctga

SEQ ID NO: 32 - protein, 72 aa, E. coli Nissle 1917
MAKEDNIEMQGTVLETLPNTMFRVELENGHVVTAHISGKMRKNYIRILTGDKVTVELTPYDLSKGRIVFRSR

SEQ ID NO: 33 - DNA, 1000 bp, E. coli Nissle 1917
  misc_feature (1)..(1000): infA knockout
ttaacaacggactaaacgtgtagtatttgagttcactgccgtacaggcagcttagaaaggaacaatcttgcaaaggctgtgaaagttggcgttcactgccgtacaggcagataaaatgcgaaaaaaactcgtactttcgtacgagctcttctttgaatatggcggtgagggggggattcgaacccccgatacgttgccgtatacacactttccaggcgtgctccttcagccactcggacacctcaccaaattgttttgctaccaaacctcatgggtggcaacggggcgctactatagggagttggagtaaaacggtcaagaagaattttaatgataattattgtttgctcatactgtaaacaagttgtgcagtatatctacatcgagacaagttatggacttatacttccaaagtacttcatacatatcacaaaataaaaaggccggttaaaccgaccttttactcgttctttctcttcgcccatcaggcggtaaaacaactaatcctctggggtatcactaccgtaatttgaaccggcaagataatgccgaagttctgtaaataagtaaagatttgcgcgctaaatcgcaacaaacaggttcggcacattactccgaaaacacacggctaagccgcaccaaaagcgcaacgtataagggagcggtgagataaacgataggcgttacctgacgcgaaaaattccttatcggcagcggggtaatgagcgtaaccaactctgcgaccgcaattataacactctggggagaaatgtgccgaaaacattcattcttgtggtgaaaacaagcaccgtggtacccagaaattattcggcaatcgtccgaggcgcatttgattgagataatttagataatcccggcggggaatttcgcaggcaccaagcgaagctgtgtgatcgttaaggacctggcagtcgataagtttaccgccatgaccaataaattcctcacagaagaccagtagcgccgttttggacgcgtt

SEQ ID NO: 34 - DNA, 3044 bp, E. coli Nissle 1917
  misc_feature (1)..(3044): Tn7_GFP
aatgtctcctgggaggattcataaagcattgtttgttggctacgagaagcaaaataggacaaacaggtgacagttatatgtaaggtttatgacagttttatgacagagagagaatgtcttcagtctgatttaaataagcgttgatattcagttaattacaaatattaataacgaagagatgacagaaaaattttcattctgtgacagagaaaaagtagccgaagatgacggtttgtcacatggagttggcaggatgtttgattaaaaacataacaggaagaaaaatgccccgctgtgggcggacaaaatagttgggaactgggaggggtggaaatggagtttttaaggattatttagggaagagtgacaaaatagatgggaactgggtgtagcgtcgtaagctaatacgaaaattaaaaatgacaaaatagtttggaactagatttcacttatctggttggtcgacactagtattaccctgttatccctagatttaaatgatatcggatcctagtaagccacgttttaattcttcacctcgagtttacacctagctcagtcctaggtattatgctagctactagaggaattcattaaagaggagaaaggtaatgagcaaaggagaagaacttttcactggagttgtcccaattcttgttgaattagatggtgatgttaatgggcacaaattttctgtcagtggagagggtgaaggtgatgctacatacggaaaactcacccttaaatttatttgcactactggaaaactacctgttccatggccaacacttgtcactactctgacctatggtgttcaatgcttttcccgttatccggatcacatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaagggtattgattttaaagaagatggaaacattctcggacacaaactagagtacaactataactcacacaatgtatacatcacggcagacaaacaaaagaatggaatcaaagctaacttcaaaattcgccacaacattgaagatggttccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaatgatagaggcatcaaataaaacgaaaggctcagtcgaaagactgggcctttcgttttatttgatgcctggttattattatttgtacagctcatccatgccaccggtagaatgacgaccctccgcgcgctcatattgctctacgatcgtattggtgagaatccaggggtccccaataattacgatttaaatttgacagcttatcatcgataaactgtaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcacttgcgttgatgcaatttctatgcgcacccgttctcggagcactgtccgaccgctttggccgccgcccagtcctgctcgcttcgctacttggagccactatcgactacgcgatcatggcgaccacacccgtcctgtggattctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccctgcacgcaccattccttgcggcggcggtgctcaacggcctcaacctactactgggctgcttcctaatgcaggagtcgcataagggagagcgccgtccgatgcccttgagagccttcaacccagtcagctccttccggtgggcgcggggcatgaccattgtggccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgagacaattgtccttttccgctgcataacctcgagcacctaggaggcgcgccacggccgcgtcgaccccacgcccctctttaatacgacgggcaatttgcacttcagaaaatgaagagtttgctttagccataacaaaagtccagtatgctttttcacagcataactggactgatttcagtttacaactattctgtctagtttaagactttattgtcatagtttagatctattttgttcagtttaagactttattgtccgcccacattacgcagggcatccattta

SEQ ID NO: 35 - DNA, 3177 bp, E. coli NIssle 1917
  misc_feature (1)..(3177): pMUT1 plasmid (Genbank A84793.1)
agcttttagagcttggataccatgacccaatgaagctacctcaaaactttgaatgatcgagcggcaggctaaatgaaatcttgagattcattcagtctcgtcgtaatctcactattgtaaaaacgaaaaaaccaccctggcaggtggtttttcgaaggttagttaatcctggcagattctctaaccgtggtaacagtcttgtgcgagacatgtcaccaaatactgtcctttcagtgtagcctcagttaggccgccacttcaagaactctcgttacatctctcgcacatcctgcttaccagtggccgttgccagtggcgttaagtcgtgtcttaccgggttggactcaagacgatagttaccggataaggcgccaggtcgggctgaacggggggttcgtgcacacagcccagcttggagcgaacgacctacaccgaacctgagatacctaacagcgtgacgtatgagaaagcgccacgcttcccgaagagaaaggcggacaggtatccggtaagcggcagggtcggaacaggagagcgcacgagggagcttccagggggaaacgcctggtatctttatatagtcctgtcgggtttcgccacctctgacttgagcgtcgatttttgtgatgctcgtcaggggggcggagcctatggaaaaagcctcccgcggagaccccttcttctgggatctttgtcttttgctcacatgttctttccggttttatcccccgattctgtggataaccgtattaccgcctttgagtgagctgacaccgctcgccgcagtcgaacgaccgagcgtagcgagtgagtgagcgaggaagcggaagagagaatttatgtgacattttctccttacgctcctctatgccgttctgcatcctgtccggatgcgttatatcccggtaagattttccgcttcaaagcgtgtctgtatgctgttctggagttcttctgcgagttcgtgcagtttctcacacatggcggcctgttcgtcggcattgagtgcgtccagtttttcgagcagcgtcaggctctgactttttatgaatcccgccatgttgagtacggcttgctgctgcttattcatcttttcgttttctccgttctgtctgtcatctgcgttgtgtgattatatcgcgtaccacttttcgactgttttgctgccgctattctgccgcttggctttttgacgggcatttctgtcagacaacactgtcactgccaaaaaactgccgtgcctttgtcggtaattcgagcttgctgacaggacaggatgtgcaattgttataccgcgcatacatgcacgctattacaattgccctggtcaggctttgccccgacacccatgtcagatacggagccatgttttatgacaaaacgaagtggaagtaatacgcgcaggcgggctatcagtcgccctgttcgtctgacggcagaagaagaccaggaaatcagaaaaagggctgctgaatgcggcaagaccgtttccggtttttttcgggcggcagctctcggtaagaaagttaactcactgactgatgatcgggtactgaaagaagttatgagactgggggcgttacagaaaaaactctttatcgacggcaagcgtgtcggggacagggagtatgcggaggtgctgatcgctattacggagtatcaccgtgccctgttatccaggcttatggcagattagcttcccggagagaaaactgtcgaaaactgacggtatgaacaccgtaagctcccaaagtgatcaccattcgctttcatgcatagctatgcagcgagctgaaacgatcctgacgcatccttcctgtttttccggggtaaaacatctctttttgcggtgtctcgcgtcagaatcgcgttcagcgcgtttcagtggtgcgtacaattaagggattatggtaaatatatgagctatgcgataactttaactgtgaagcgatgaacccattacaggcaaagccaattactcctgacagtggtttagccagaagcagggctaccaagaccaatgcaataagtaatatatcgttttgctatcgtgccatccgtcgcgctcagttccattgtgcttttttaagctgtcgtttttcttacggtatataccggttttttatggcgtggtttcttaacttgttcagctactgatggacccatgtatctaggtagtcaactagctttgttagatcataaaatattgcgaccaccatatcggcgatcactcttcgatgttggtttcttgtccaagagattagctttttcaagatcattgatagctctctgaacagtccgtacagaaacccccatacgtatggctagactttccattgacggatgcggccactcttgcaaactccaccagtgaacgatcaggttaagtagtgtgttaaaggccactgaagttagctttttctcgttttgtataaaaaacaatacggtaggcactgctgtccagccaagagacaaaccgccagctttccatttattcttaacggagtaagtcattgattttcctaagccccaaaatatttaaagtatatattatatgtatattcatatgaatagggtgacactggcgccattattgtgcaaccaaaaaagactactctgaaaacgaggaaaagattttttcctgcctgaattagatacggagttagcgatatgaaaaccgaacaacgtcatgatcttgttaaagatattgaggtttttggcgtatccttgtctctgttgatttccagagcgaatgagaagtctgttacaatgccatctggtctaagtcgggagcagagaagagcatgggcagcggagcaggcgcgcaaaatccacaattgaatattgtctcattctctgagaccttcaacctttattacacatccagatattctgcaaaaacactcgataaaatcgatgatttcattgagcattttgaaaaatacaatctctttggcgatcctttaaaaggatatccagcttggactggcaaagtatcgccatcgtggaaagtgcctgatcattacgaaaacaaagaagctattgagaagtatgctagagctaacaaattatggcatgctcatttaggcgatccggtttttaaagatacgtttcatgggaaatacaaggtttctgactgggttattcatttccagcggctgacaccgaaccatataa

SEQ ID NO: 36 - DNA, 35 bp, Artificial Sequence
  Synthetic
  promoter (1)..(35): promoter BBa_J23101
tttacagctagctcagccctaggtattatgctagc

SEQ ID NO: 37 - DNA, 35 bp, Artificial Sequence
  Synthetic
  promoter (1)..(35): promoter BBa_J23107
tttacggctagctcagccctaggtattatgctagc

SEQ ID NO: 38 - DNA, 50 bp, Artificial Sequence
  Synthetic
  RBS (1)..(50): M1 RBS=63000
aggtattatgctagcagctcatcaatacccccccaataaggaggattaag

SEQ ID NO: 39 - DNA, 50 bp, Artificial Sequence
  Synthetic
  rbs (1)..(50): M1 RBS=35000
aggtattatgctagcagctcatcaatacccccccaacaaggaggattaag

SEQ ID NO: 40 - DNA, 35 bp, Artificial Sequence
  Synthetic
  rbs (1)..(35): M1 RBS=1800
agctcatcaatacccccccaataaggaggattaag

SEQ ID NO: 41 - DNA, 50 bp, Artificial Sequence
  Synthetic
  rbs (1)..(50): M1 RBS=13000
aggtattatgctagcagctcatcaatacccccccaatcaggaggattaag

SEQ ID NO: 42 - DNA, 50 bp, Artificial Sequence
  Synthetic
  rbs (1)..(50): M1 RBS=4200
aggtattatgctagcagctcatcaatacccccccaaccaggaggattaag

SEQ ID NO: 43 - DNA, 35 bp, Artificial Sequence
  Synthetic
  rbs (1)..(35): M1 RBS=2300
agctcatcaatacccccccaaaaataaggattaag

SEQ ID NO: 44 - DNA, 35 bp, Artificial Sequence
  Synthetic
  rbs (1)..(35): H2 RBS=36000
agctcatcaatacccccccataagcgaggtttaag

SEQ ID NO: 45 - DNA, 35 bp, Artificial Sequence
  Synthetic
  rbs (1)..(35): H2 RBS=10000
agctcatcaatacccccccataaccgaggtttaag

SEQ ID NO: 46 - DNA, 35 bp, Artificial Sequence
  Synthetic
  rbs (1)..(35): H2 RBS=5200
agctcatcaatacccccccataagcaaggtttaag

SEQ ID NO: 47 - DNA, 35 bp, Artificial Sequence; identical to SEQ ID NO: 43
  Synthetic
  rbs (1)..(35): H2 RBS=5000

SEQ ID NO: 48 - DNA, 35 bp, Artificial Sequence
  Synthetic
  rbs (1)..(35): H2 RBS=3000
agctcatcaatacccccccataaccaaggtttaag

SEQ ID NO: 49 - DNA, 32 bp, Artificial Sequence
  Synthetic
  rbs (1)..(32): R RBS=10000
gaagaaattatcagagagagggaaggtaacac

SEQ ID NO: 50 - DNA, 45 bp, Artificial Sequence
  Synthetic
  rbs (1)..(45): R RBS=3800
tacccgtagctaagaagaaattatcagagagagggtaggtaacac

SEQ ID NO: 51 - DNA, 45 bp, Artificial Sequence
  Synthetic
  rbs (1)..(45): R RBS=1800
tacccgtagctaagaagaaattatcagagagacggtaggtaacac

SEQ ID NO: 52 - DNA, 45 bp, Artificial Sequence
  Synthetic
  rbs (1)..(45): R RBS=700
tacccgtagctaagaagaaattatcagagagagggtcggtaacac

SEQ ID NO: 53 - DNA, 35 bp, Artificial Sequence
  Synthetic
  rbs (1)..(35): R RBS=250
aagctaagaagaaattatcagagggtcggtaacac

SEQ ID NO: 54 - DNA, 45 bp, Artificial Sequence
  Synthetic
  rbs (1)..(45): R RBS=50
tacccgtagctaagaagaaattatcagagagacggtcggtaacac

SEQ ID NO: 55 - DNA, 22 bp, Artificial Sequence
  Synthetic
  primer (1)..(22): CYCLO forward primer
gacgaaggtagccagtcacaag

SEQ ID NO: 56 - DNA, 22 bp, Artificial Sequence
  Synthetic
  primer (1)..(22): CYCLO revserse primer
aatcaggcctgtggaatgtgag

SEQ ID NO: 57 - DNA, 20 bp, Artificial Sequence
  Synthetic
  primer (1)..(20): MUC2 forward primer
atgcccacctcctcaaagac

SEQ ID NO: 58 - DNA, 23 bp, Artificial Sequence
  Synthetic
  primer (1)..(23): MUC2 reverse primer
gtagtttccgttggaacagtgaa

SEQ ID NO: 59 - DNA, 21 bp, Artificial Sequence
  Synthetic
  primer (1)..(21): CDX forward primer
caaggacgtgagcatgtatcc

SEQ ID NO: 60 - DNA, 21 bp, Artificial Sequence
  Synthetic
  primer (1)..(21): CDX2 reverse primer
gtaaccaccgtagtccgggta

SEQ ID NO: 61 - DNA, 23 bp, Artificial Sequence
  Synthetic
  primer (1)..(23): BM1 forward primer
aaatccccacttaatgtgtgtcc

SEQ ID NO: 62 - DNA, 21 bp, Artificial Sequence
  Synthetic
  primer (1)..(21): BM1 reverse primer
cttgctggtctccaagtaacg

SEQ ID NO: 63 - DNA, 19 bp, Artificial Sequence
  Synthetic
  primer (1)..(19): LGR5 forward primer
ggaccagatgcgataccgc

SEQ ID NO: 64 - DNA, 21 bp, Artificial Sequence
  Synthetic
  primer (1)..(21): LGR5 reverse primer
cagaggcgatgtaggagactg

SEQ ID NO: 65 - DNA, 21 bp, Artificial Sequence
  Synthetic
  primer (1)..(21): IL6 forward primer
ctgcaagagacttccatccag

SEQ ID NO: 66 - DNA, 23 bp, Artificial Sequence
  Synthetic
  primer (1)..(23): IL6 reverse primer
agtggtatagacaggtctgttgg

SEQ ID NO: 67 - DNA, 22 bp, Artificial Sequence
  Synthetic
  primer (1)..(22): IL1beta forwrad primer
gcaactgttcctgaactcaact

SEQ ID NO: 68 - DNA, 21 bp, Artificial Sequence
  Synthetic
  primer (1)..(21): IL1beta reverse primer
atcttttggggtccgtcaact

SEQ ID NO: 69 - DNA, 21 bp, Artificial Sequence
  Synthetic
  primer (1)..(21): NFKbeta forward primer
atggcagacgatgatccctac

SEQ ID NO: 70 - DNA, 23 bp, Artificial Sequence
  Synthetic
  primer (1)..(23): NFKbeta reverse primer
tgttgacagtggtatttctggtg

SEQ ID NO: 71 - DNA, 21 bp, Artificial Sequence
  Synthetic
  primer (1)..(21): IL17A forward primer
tttaactcccttggcgcaaaa

SEQ ID NO: 72 - DNA, 20 bp, Artificial Sequence
  Synthetic
  primer (1)..(20): IL17A reverse primer
ctttccctccgcattgacac

SEQ ID NO: 73 - DNA, 19 bp, Artificial Sequence
  Synthetic
  primer (1)..(19): TNFalpha forward primer
caggcggtgcctatgtctc

SEQ ID NO: 74 - DNA, 22 bp, Artificial Sequence
  Synthetic
  primer (1)..(22): TNFalpha reverse primer
cgatcaccccgaagttcagtag

SEQ ID NO: 75 - DNA, 23 bp, Artificial Sequence
  Synthetic
  primer (1)..(23): TPH1 forward primer
aacaaagaccattcctccgaaag

SEQ ID NO: 76 - DNA, 23 bp, Artificial Sequence
  Synthetic
  primer (1)..(23): TPH1 reverse primer
tgtaacaggctcacatgattctc

SEQ ID NO: 77 - DNA, 19 bp, Artificial Sequence
  Synthetic
  primer (1)..(19): TPH2 forward primer
gtgaccctgaatccgcctg

SEQ ID NO: 78 - DNA, 19 bp, Artificial Sequence
  Synthetic
  primer (1)..(19): TPH2 reverse primer
ggtgccgtacatgaggact

SEQ ID NO: 79 - DNA, 21 bp, Artificial Sequence
  Synthetic
  primer (1)..(21): SERT forwrad primer
ctccgcagttcccagtacaag

SEQ ID NO: 80 - DNA, 21 bp, Artificial Sequence
  Synthetic
  primer (1)..(21): SERT reverse primer
cacggcatagccaatgacaga

SEQ ID NO: 81 - DNA, 20 bp, Artificial Sequence
  Synthetic
  primer (1)..(20): CHGA forward primer
caggctacaaagcgatccag

SEQ ID NO: 82 - DNA, 20 bp, Artificial Sequence
  Synthetic
  primer (1)..(20): CHGA reverse primer
gcctctgtctttccatctcc

SEQ ID NO: 83 - DNA, 19 bp, Artificial Sequence
  Synthetic
  primer (1)..(19): TJP1 forward primer
gccgctaagagcacagcaa

SEQ ID NO: 84 - DNA, 21 bp, Artificial Sequence
  synthetic
  primer (1)..(21): TJP1 reverse primer
tccccactctgaaaatgagga

SEQ ID NO: 85 - DNA, 21 bp, Artificial Sequence
  Synthetic
  primer (1)..(21): TJP2 forward primer
gtttgccgttcagcagcttag

SEQ ID NO: 86 - DNA, 21 bp, Artificial Sequence
  Synthetic
  primer (1)..(21): TJP2 reverse primer
cttcaaaacctcggtcgtcat

SEQ ID NO: 87 - DNA, 22 bp, Artificial Sequence
  Synthetic
  primer (1)..(22): OCLN forward primer
tgaaagtccacctccttacaga

SEQ ID NO: 88 - DNA, 22 bp, Artificial Sequence
  Synthetic
  primer (1)..(22): OCLN reverse primer
ccggataaaaagagtacgctgg

SEQ ID NO: 89 - DNA, 22 bp, Artificial Sequence
  Synthetic
  primer (1)..(22): IDO1 forward primer
caaagcaatccccactgtatcc

SEQ ID NO: 90 - DNA, 23 bp, Artificial Sequence
  Synthetic
  primer (1)..(23): IDO1 reverse primer
acaaagtcacgcatcctcttaaa

SEQ ID NO: 91 - DNA, 20 bp, Artificial Sequence
  Synthetic
  primer (1)..(20): IDO2 forward primer
cctcatccctccttcctttc

SEQ ID NO: 92 - DNA, 20 bp, Artificial Sequence
  Synthetic
  primer (1)..(20): IDO2 reverse primer
ggagcaattgcctggtatgt

SEQ ID NO: 93 - DNA, 23 bp, Artificial Sequence
  Synthetic
  primer (1)..(23): TDO2 forward primer
aggaacatgctcaaggtgatagc

SEQ ID NO: 94 - DNA, 22 bp, Artificial Sequence
  Synthetic
  primer (1)..(22): TDO2 reveres primer
ctgtagactctggaagcctgat

SEQ ID NO: 95 - DNA, 19 bp, Artificial Sequence
  Synthetic
  primer (1)..(19): HTR1b forward primer
cgccgacggctacatttac

SEQ ID NO: 96 - DNA, 20 bp, Artificial Sequence
  Synthetic
  primer (1)..(20): HTR1b reverse primer
tagcttccgggtccgataca

SEQ ID NO: 97 - DNA, 20 bp, Artificial Sequence
  Synthetic
  primer (1)..(20): HTR1d forward primer
atcaccgatgccctggagta

SEQ ID NO: 98 - DNA, 20 bp, Artificial Sequence
  Synthetic
  primer (1)..(20): HTR1d reverse primer
gcgagaagagtggagggatg

SEQ ID NO: 99 - DNA, 22 bp, Artificial Sequence
  Synthetic
  primer (1)..(22): HTR3 forward primer
cctggctaactacaagaagggg

SEQ ID NO: 100 - DNA, 23 bp, Artificial Sequence
  Synthetic
  primer (1)..(23): HTR3 reverse primer
tgcagaaactcatcagtccagta

SEQ ID NO: 101 - DNA, 21 bp, Artificial Sequence
  Synthetic
  primer (1)..(21): HTR4 forward primer
agttccaacgagggtttcagg

SEQ ID NO: 102 - DNA, 19 bp, Artificial Sequence
  Synthetic
  primer (1)..(19): HTR4 reverse primer
cagcaggttgcccaagatg

SEQ ID NO: 103 - DNA, 20 bp, Artificial Sequence
  Synthetic
  primer (1)..(20): HTR7 forward primer
tgcggggagcagatcaacta

SEQ ID NO: 104 - DNA, 21 bp, Artificial Sequence
  Synthetic
  primer (1)..(21): HTR7 reverse primer
gacaaagcacaccgagatcac

SEQ ID NO: 105 - DNA, 50 bp, Bacillus subtilis
  promoter (1)..(50): Bacillus subtilis P_PS1
actgcgtcaatacacgttgacactcttttgaaagtgtgttaaattatcag

SEQ ID NO: 106 - DNA, 24 bp, Bacillus subtilis
  RBS (1)..(24): Bacillus subtilis R0 (RBS)
gattaactaataaggaggacaaaa

SEQ ID NO: 107 - DNA, 21 bp, Bacillus subtilis
  RBS (1)..(21): Bacillus subtilis R1 (RBS)
gctcttaaggaggattttaga

SEQ ID NO: 108 - DNA, 428 bp, S. cerevisiae
  promoter (1)..(428): S. cerevisiae promoter P_TEF1
gcacacaccatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaagtgcaggt

SEQ ID NO: 109 - DNA, 992 bp, S. cerevisiae
  promoter (1)..(992): S. cerevisiae promoter P_PKG1
ggaagtaccttcaaagaatggggtcttatcttgttttgcaagtaccactgagcaggataataatagaaatgataatatactatagtagagataacgtcgatgacttcccatactgtaattgcttttagttgtgtatttttagtgtgcaagtttctgtaaatcgattaatttttttttctttcctctttttattaaccttaatttttattttagattcctgacttcaactcaagacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatctgtcat

SEQ ID NO: 110 - DNA, 1440 bp, Candidatus Koribacter versatilis Ellin345
  CDS (1)..(1440): Candidatus Koribacter versatilis Ellin345 TDC tryptophan decarboxylase (EC 4.1.1.28)
atgaaccgcatgaaaaacaactttcacatgctgcctgatgattttcgtgcagcaggtcataaagttattgattgggttgcagattatcatgcccacgttgaagattttcgcgttctgagccaggttaaaccgggtgaaatttgtgatggtctgccggatagccctccgcagcagggtgatagcgttaccaatattctgcctgatattgaacgtcatgttctgccaggtattacccattggcagagcccgaacttttatgcatattttccgagcaataattccggtccgagcattctgggtgatctggttagcagcggtctgggtgttcagggtatgctgtgggcaaccagtccggcatgtaccgaagttgaaatgaaaatgctggattggctggttcagatgctgggcctgccggaacattttctgaatagcagcaaacatggtggtggtgttattcaggatagcgcaagcagcgcaaccctgtgtgcactgctggcagcacgtgaacaggcaaccaatggtcagaccaatgaagaaggttgtcgtctgccgctggtttgttataccagcaatcaggcacatagccatgttgagaaagatgttaaagttgcaggtctgggtcgtaaaaatctgcgtctgattgatgtggatcaagaatttgcaatgcgtccggaagcactggaacgtcagattgttgaagataaagcagcaggcaaaatcccgttttttgtttgtgcaaccattggtacaaccagcagcctggcaattgatccgattccggaaattgcagccatttgtaaacgccatggtctgtggctgcatgttgatgcagcaatggcaggcaccgcagcactgtgtccggaatttcgttggacccataatggtgttgaactggcagatagctatgcatttaatccgcacaaatggatgtataccaactttgattgtaccgccttttgggtgaaagatcgtcatgcactgattaatagcctgagcgttgttccggaatatctgcgtaatcaggcaagcgaacagggtgaagtttttgattatcgtgattggcatgttccgctgggtcgtcgttttcgtgccctgaaactgtggtttgtgattcgtcattatggtgtggaaggtctgcagcatcatgttcgtcagaatgttgcatgggcacaagaattcgcagcatgggttaaagcagatagccgttttgaactggttgcaccgcatccgctgagcctggtttgctttcgtctgaaaagcggtgatgcagccagcgaacagctgctgaaacgtgcaaatgaaagcggcaaaatctttatcagccataccaaactggatggcaaatatgttctgcgttttagcattggtcaggcaaaaaccgaacgtcatcatgttgaagccgcatggaaactgattagcgatctggccgatcgtagctaa

SEQ ID NO: 111 - protein, 479 aa, Candidatus Koribacter versatilis Ellin345
MNRMKNNFHMLPDDFRAAGHKVIDWVADYHAHVEDFRVLSQVKPGEICDGLPDSPPQQGDSVTNILPDIERHVLPGITHWQSPNFYAYFPSNNSGPSILGDLVSSGLGVQGMLWATSPACTEVEMKMLDWLVQMLGLPEHFLNSSKHGGGVIQDSASSATLCALLAAREQATNGQTNEEGCRLPLVCYTSNQAHSHVEKDVKVAGLGRKNLRLIDVDQEFAMRPEALERQIVEDKAAGKIPFFVCATIGTTSSLAIDPIPEIAAICKRHGLWLHVDAAMAGTAALCPEFRWTHNGVELADSYAFNPHKWMYTNFDCTAFWVKDRHALINSLSVVPEYLRNQASEQGEVFDYRDWHVPLGRRFRALKLWFVIRHYGVEGLQHHVRQNVAWAQEFAAWVKADSRFELVAPHPLSLVCFRLKSGDAASEQLLKRANESGKIFISHTKLDGKYVLRFSIGQAKTERHHVEAAWKLISDLADRS

SEQ ID NO: 112 - DNA, 72 bp, Artificial Sequence
  Synthetic
  promoter (1)..(72): trc promoter
ttgacaattaatcatccggctcgtataatgtgtggaattgtgagcggataacaatttcacacaggagtaaaa